(19) United States
(12) Patent Application Publication
(10) Pub. No.: US 2008/0161248 A1
(43) Pub. Date: Jul. 3, 2008

(54) Methods and Compositions for Therapeutic Treatment

(76) Inventor: Wendye Robbins, South San Francisco, CA (US)

Correspondence Address: WILSON SONSINI GOODRICH & ROSATI, 650 PAGE MILL ROAD, PALO ALTO, CA, 94304-1050, US

(21) Appl. No.: 11/964,377

(22) Filed: Dec. 26, 2007

Related U.S. Application Data
(60) Provisional application No. 60/882,306, filed on Dec. 28, 2006.
(60) Provisional application No. 60/940,375, filed on May 25, 2007.
(60) Provisional application No. 60/953,192, filed on Jul. 31, 2007.

(51) Int. Cl. A61K 31/7048; A61K 31/436; A61P 37/00; A61P 29/00; A61K 31/352

(52) U.S. Cl. 514/27; 514/291; 514/456

(57) ABSTRACT

Methods and compositions are described for the modulation of central nervous system and/or fetal effects of calcineurin inhibitors. Methods and compositions are described for the modulation of efflux transporter activity to increase the efflux of calcineurin inhibitors out of a physiological compartment and into an external environment. In particular, the methods and compositions disclosed herein provide for the increase of efflux transporter activity at Blood-Tissue, blood-CSF and placental-maternal barriers to increase the efflux of calcineurin inhibitor from physiological compartments, including central nervous system and fetal compartments.

### CROSS-REFERENCE

This application claims the benefit of U.S. Provisional Application No. 60/882,306, filed Dec. 28, 2006; U.S. Provisional Application No. 60/940,375, filed May 25, 2007; and U.S. Provisional Application No. 60/953,192, filed Jul. 31, 2007, which are incorporated herein by reference in their entirety.

### BACKGROUND OF THE INVENTION

Although anatomical blood barrier structures, such as the Blood-Tissue barrier (BTB), function as a block, for example, to isolate the central nervous system from the systemic blood circulation, pharmaceutical agents often cross the barrier causing systemic side-effects rather than a desired localized action.

For instance, Prograf, the market leading immunosuppressant for preventing transplant rejection has been reported to caused neurotoxicity, including tremor, headaches, and other changes in motor function, mental status, and sensory function, in approximately 55% or liver transplant recipients. Tremor occurred in 54% of Prograf-treated kidney transplant patients and 15% of heart transplant patients. Other immunosuppressants, such as Cyclosporin, also cause neurotoxicity that leads to undesirable side effects. Prograf has also been shown to cause, can cause acute and chronic nephrotoxicity. As solid organ transplantation is increasing and grafts last longer than they used to—a kidney given in 2003 is expected to last 20 years—there is a need to find methods to decrease side effects that impinge on quality of life of patients.

### SUMMARY OF THE INVENTION

The invention provides methods, compositions, and kits for the use of blood-tissue barrier (BTB) transport protein modulator, e.g., to reduce or eliminate a side effect and/or enhance a therapeutic effect of a calcineurin inhibitor.

In one aspect the invention provides compositions including a BTB transport protein modulator. In some embodiment, the invention provides compositions including an effective amount of a calcineurin inhibitor and an amount of a BTB transport protein modulator sufficient to decrease a side effect of the calcineurin inhibitor. In some embodiments of this aspect, the invention provides a composition including a calcineurin inhibitor and a Blood-Tissue barrier (BTB) transport protein modulator, where the BTB transport protein modulator is present in an amount sufficient to enhance a therapeutic effect of the calcineurin inhibitor when the composition is administered to an animal. In some embodiments of this aspect, the invention provides a composition including a calcineurin inhibitor and a Blood-Tissue barrier (BTB) transport protein modulator, where the BTB transport protein modulator is present in an amount sufficient to increase the concentration of the calcineurin inhibitor in a physiological compartment when the composition is administered to an animal. In some embodiments of the compositions of the invention, the physiological compartment includes blood, lymph nodes, spleen, peyer's patches, lungs, and heart.

In some embodiments of this aspect, BTB transport protein includes an ABC transport protein. In some embodiments of the composition, the BTB transport protein modulator in the composition includes a BTB transport protein activator. In some embodiments, the BTB transport protein modulator in the composition includes a modulator of P-gP. In some embodiments, the BTB transport protein modulator in the composition includes a polyphenol. In some embodiments of the invention, the polyphenol includes a flavonoid. In some embodiments, the polyphenol includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments, the flavonoid is quercetin.

In some embodiments of the compositions of the invention, the BTB transport protein modulator decreases a side effect. In some embodiments of the compositions of the invention, the BTB transport protein modulator decreases a central nervous system (CNS) effect. In some embodiments, the CNS effect is selected from the group consisting of tremors, headache, changes in motor function, changes in mental status, changes in sensory functions, seizures, insomnia, paresthesia, dizziness, coma and delirium. In some embodiments of the compositions of the invention, the BTB transport protein modulator decreases a hepatic, pancreatic and/or gastrointestinal side effect. In some embodiments, the hepatic, pancreatic and/or gastrointestinal side effect is hepatic necrosis, hepatotoxicity, liver fatty, venooclusive liver disease, diarrhea, nausea, constipation, vomiting, dyspepsia, anorexia, or LFT abnormal. In some embodiments of the compositions of the invention, the BTB transport protein modulator decreases a renal and/or urogenital side effect. In some embodiments, the renal and/or urogenital side effect side effect is nephrotoxicity, renal function impairment, creatinine increase, urinary tract infection, oliguria, cystitis haemorrhagic, hemolytic-uremic syndrome or micturition disorder. In some embodiments, the side effect is decrease in tissue metabolic function.

In some embodiments of the compositions of the invention, a pharmaceutical composition includes the composition of the invention and a pharmaceutically acceptable excipient. In some embodiments of the composition, a molar ratio of the calcineurin inhibitor and the BTB transport protein modulator is about 0.001:1 to about 0.10:1. In some embodiments of the composition, the calcineurin inhibitor is present in an amount of about 0.1-1000 mg and the BTB transport protein modulator is present in an amount of about 10 to 1000 mg. In some embodiments of the invention, a kit includes the composition of the invention and instructions for use of the composition.

In another aspect, the invention provides methods utilizing BTB transport protein modulator. In some embodiments of this aspect, the invention provides a method of treating a condition by administering to an animal suffering from the condition an effective amount of a calcineurin inhibitor and an amount of a BTB transport protein modulator sufficient to increase a therapeutic effect of the calcineurin inhibitor. In some embodiments of the methods of the invention, the therapeutic effect of calcineurin inhibitor is increased at least about 5% compared to the therapeutic effect without the BTB transport protein modulator, when the composition is administered to an animal.

In some embodiments of the methods of the invention the BTB transport protein modulator is a BTB protein transport activator. In some embodiments of this aspect, the invention provides a method of treating a condition by administering to an animal suffering from the condition an effective amount of a calcineurin inhibitor and an amount of a BTB transport protein modulator sufficient to reduce or eliminate a CNS effect of the calcineurin inhibitor. In some embodiments, the modulator reduces or eliminates a plurality of CNS effects of the calcineurin inhibitor. In some embodiments of this aspect, the invention provides a method of treating a condition by administering to an animal suffering from the condition an effective amount of a calcineurin inhibitor and an amount of a BTB transport protein modulator sufficient to reduce or eliminate a hepatic, pancreatic and/or gastrointestinal side effect of the calcineurin inhibitor. In some embodiments, the modulator reduces or eliminates a plurality of hepatic, pancreatic and/or gastrointestinal side effects of the calcineurin inhibitor. In some embodiments of this aspect, the invention provides a method of treating a condition by administering to an animal suffering from the condition an effective amount of a calcineurin inhibitor and an amount of a BTB transport protein modulator sufficient to reduce or eliminate a renal and/or urogenital side effect of the calcineurin inhibitor. In some embodiments, the modulator reduces or eliminates a plurality of renal and/or urogenital side effects of the calcineurin inhibitor. In some embodiments, the calcineurin inhibitor and the BTB transport protein modulator are administered in a single composition. In some embodiments, the calcineurin inhibitor and the BTB transport protein modulator are admixed in the composition.

In some embodiments of the methods of the invention, the calcineurin inhibitor is present in an amount sufficient to exert a therapeutic effect and the BTB transport protein activator is present in an amount sufficient to decrease a side effect of the calcineurin inhibitor by an average of at least about 5%, compared to the effect without the BTB transport protein activator. In some embodiments, the administration is oral administration. In some embodiments of the methods of the invention, the side effect is a CNS side effect. In some embodiments, the CNS side effect is selected from the group consisting of tremors, headache, changes in motor function, changes in mental status, changes in sensory functions, seizures, insomnia, paresthesia, dizziness, coma and delirium. In some embodiments of the methods of the invention, the BTB transport protein modulator decreases a hepatic, pancreatic and/or gastrointestinal side effect. In some embodiments, the hepatic, pancreatic and/or gastrointestinal side effect is hepatic necrosis, hepatotoxicity, liver fatty, venooclusive liver disease, diarrhea, nausea, constipation, vomiting, dyspepsia, anorexia, or LFT abnormal. In some embodiments of the methods of the invention, the BTB transport protein modulator decreases a renal and/or urogenital side effect. In some embodiments, the renal and/or urogenital side effect side effect is nephrotoxicity, renal function impairment, creatinine increase, urinary tract infection, oliguria, cystitis haemorrhagic, hemolytic-uremic syndrome or micturition disorder. In some embodiments, the side effect is a decrease in tissue metabolic function.

In some embodiments of the methods of the invention, the calcineurin inhibitor is present in an amount sufficient to exert a therapeutic effect and the BTB transport protein modulator is present in an amount sufficient to increase the concentration of the calcineurin inhibitor in a physiological compartment. In some embodiments of the methods of the invention, the physiological compartment is selected from the group consisting of blood, lymph nodes, spleen, peyer's patches, lungs, and heart.

In some embodiments of the methods of the invention, the invention provides a method of treating a condition by administering to an animal suffering from the condition an effective amount of tacrolimus and an amount of a BTB transport protein modulator sufficient to change the concentration of tacrolimus in a physiological compartment. In some embodiments of the methods of the invention the physiological compartment is selected from the group consisting of blood, lymph nodes, spleen, peyer's patches, lungs, heart kidney, pancreas liver, and gull bladder. In some embodiments of the methods of the invention the BTB transport modulator decrease the clearance of tacrolimus from a compartment where the drug is exerting therapeutic effect.

In some embodiments of the methods of the invention, the BTB transport protein modulator includes an activator of P-gP. In some embodiments, the BTB transport protein modulator includes a polyphenol. In some embodiments, the polyphenol includes a flavonoid. In some embodiments of the invention, the polyphenol includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid includes quercetin or other substituted analogs of naturally-occurring (bio)flavonoids. In some embodiments of the invention, the calcineurin inhibitor is tacrolimus or a tacrolimus analog. Examples of tacrolimus analog include meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate. In some embodiments, the calcineurin inhibitor is tacrolimus.

In some embodiments of the methods of the invention, the individual suffers from a condition including organ transplant, an autoimmune disease, and an inflammatory disease. In some embodiments the individual suffers from an organ transplant. In some embodiments, the organ transplant is selected from the group consisting of kidney transplant, pancreas transplant, liver transplant, heart transplant, lung transplant, intestine transplant, pancreas after kidney transplant, and simultaneous pancreas-kidney transplant. In some embodiments the individual suffers from an autoimmune disease. In some embodiments, the autoimmune disease is selected from the group consisting of Lupus nephritis, actopic dermatitis, rheumatoid arthritis, and psoriasis. In some embodiments the individual suffers from an inflammatory disease. In some embodiments, the inflammatory disease is selected from the group consisting of asthma, vulvar lichen sclerosis, chronic allergic contact dermatitis, eczema, vitiligo and ulcerative colitis.

In some embodiments of the method of the invention, the administration includes single or multiple doses of said calcineurin inhibitor and single or multiple doses of said BTB transport protein modulator. In some embodiments of the method of the invention, the administration comprising simultaneous administration of said calcineurin inhibitor and said BTB transport protein modulator in the same dosage form, simultaneous administration in separate dosage forms, or separate administration. In some embodiments of the method of the invention, the administration includes simultaneous administration of the calcineurin inhibitor and the BTB transport protein modulator in the same dosage form. In some embodiments of the method of the invention, the administration is oral administration.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All publications, patents, and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts blood brain barrier (BBB) and blood-cerebrospinal fluid (CSF) barrier which regulates the brain.

FIG. 2 depicts various transporters that regulate rate of brain permeation for compounds with varying lipophilicity.

FIG. 3 provides an illustration of active transporters for both influx and efflux.

FIG. 4 depicts the effect of quercetin on FK 506 inhibition of lymphocyte proliferation in a MLR response at a 0.5:1 R:S ratio (n=3).

FIG. 5 depicts the effect of quercetin on FK 506 inhibition of lymphocyte proliferation in a MLR response at a 1:1 R:S ratio (n=3).

FIG. 6 depicts the effect of quercetin on FK 506 inhibition of lymphocyte proliferation in a MLR response at a 5:1 R:S ratio (n=3).

FIG. 7 depicts the effect of quercetin on FK 506 inhibition of lymphocyte proliferation after Con A stimulation (n=3).

FIG. 8 shows the effect of quercetin and tacrolimus on response of mouse spleen cells to concanavalin A at a high cell concentration.

FIG. 9 shows the effect of quercetin and tacrolimus on response of mouse spleen cells to concanavalin A at a low cell concentration.

FIG. 10 shows the effect of quercetin and tacrolimus on response of mouse spleen cells to LPS at a high cell concentration.

FIG. 11 shows the effect of quercetin and tacrolimus on response of mouse spleen cells to LPS at a low cell concentration.

FIG. 12 shows the effect of vehicle treatment on mitogen responses at a high cell concentration.

FIG. 13 shows the effect of vehicle treatment on mitogen responses at a low cell concentration.

FIG. 14 depicts the effect of different doses of quercetin on FK 506 inhibition of lymphocyte proliferation after Con A stimulation at a high cell concentration.

FIG. 15 depicts the effect of different doses of quercetin on FK 506 inhibition of lymphocyte proliferation after Con A stimulation at a low cell concentration.

FIG. 16 depicts a table with the pharmacokinetics parameters of FK 506 in male Lewis rats after 1 mg/kg i.v. administration

FIG. 17 depicts the levels of FK 506 in plasma at different time points after quercetin administration.

FIG. 18 depicts the calculated AUC (O-infinity) of rats treated with FK 506 i.v. and Quercentin i.p. at two different concentrations of quercetin.

FIG. 19 depicts a table with non compartmental calculations of rats treated with FK 506 i.v. and Quercentin i.p. at two different concentrations of quercetin.

FIG. 20 depicts the levels of FK 506 in whole blood at different time points after i.p. administration of quercetin at two different concentrations of quercetin.

### DETAILED DESCRIPTION OF THE INVENTION

Reference will now be made in detail to particularly preferred embodiments of the invention. Examples of the preferred embodiments are illustrated in the following Examples section.

Unless defined otherwise, all technical and scientific terms used herein have the same meaning as is commonly understood by one of skill in the art to which this invention belongs. All patents and publications referred to herein are incorporated by reference.

In one aspect, the invention provides compositions and methods utilizing an agent that modulates an effect, e.g., that reduces or eliminates a side effect and/or increases a therapeutic effect associated with calcineurin inhibitor treatment. In one aspect, the invention provides compositions and methods utilizing an agent that changes the concentration of a calcineurin inhibitor in a physiological compartment. In some embodiments, the invention provides compositions and methods utilizing a combination of a calcineurin inhibitor and an agent that reduces or eliminates a side effect associated with calcineurin inhibitor treatment. In some embodiments, the invention provides compositions and methods utilizing a combination of a calcineurin inhibitor and an agent that increases or enhances a therapeutic effect associated with calcineurin inhibitor treatment. In some embodiments, the invention provides compositions and methods utilizing a combination of a calcineurin inhibitor and an agent that changes the concentration of a calcineurin inhibitor in a physiological compartment. Examples of calcineurin inhibitors include cyclosporin A (CsA), tacrolimus and tacrolimus analogs.

### Blood-Brain Barrier and Placental Barrier

A. Blood Brain Barrier

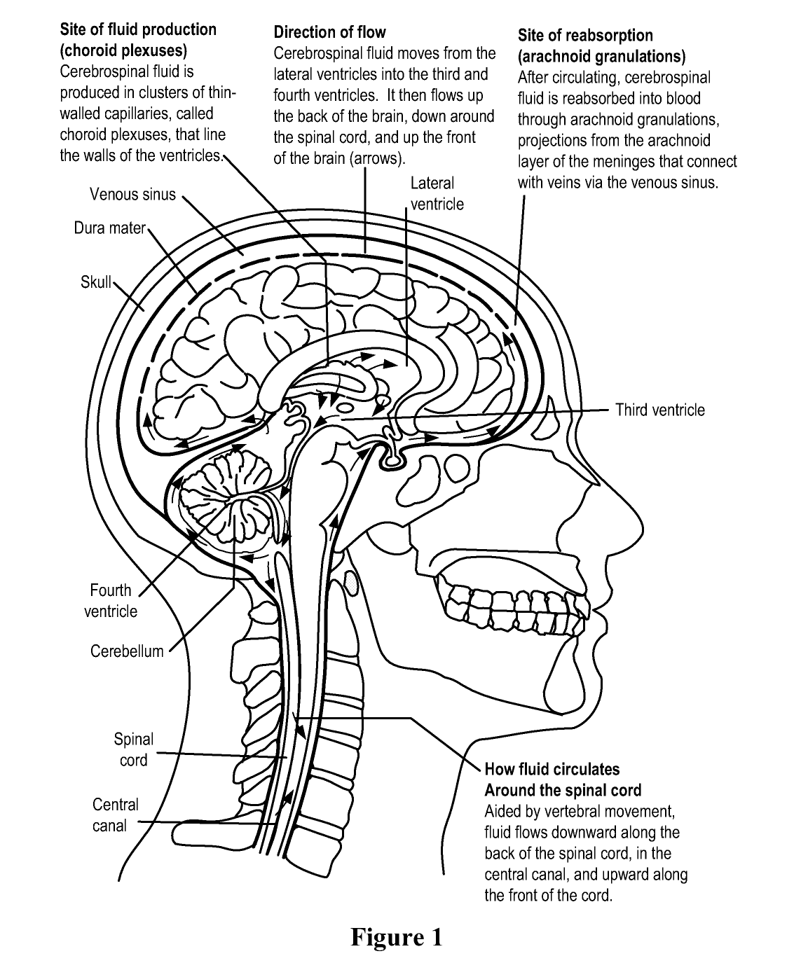

The access to the brain is controlled by at least two barriers, i.e., blood brain barrier (BBB) and blood-cerebrospinal fluid (CSF) barrier (see FIG. 1). As used herein, the term “blood brain-barrier” can encompass the blood-brain and blood-CSF barriers, unless otherwise indicated. The methods and compositions described herein are suitable for modulating the access of drugs into the brain. In some embodiments, the methods and compositions involve the modification of the blood brain barrier and/or blood-CSF barrier to prevent the entry of drugs into the central nervous system (CNS), e.g., by promoting efflux of the drugs from the CNS. In some embodiments, the compositions and methods of the invention utilize a modulator of a blood brain-barrier transport protein. In some embodiments, the compositions and methods of the invention utilize an activator of a blood brain-barrier transport protein.

The blood brain barrier is formed by tight intercellular junctions of brain capillary endothelial cells. The junctions are sealed by zonulae occludentes and tight junctions. The capillaries are covered by a continuous basal membrane enclosing pericytes, an intermittent cell layer, and the outer basal membrane is contacted by astrocytes. The electrical resistance across the endothelium is high, about 1500 to about 2000 Ω/cm².

The blood brain barrier regulates the transfer of substances between circulating blood and brain by facilitated transport and/or facilitated efflux. The interface on both luminal and abluminal surfaces contain physical and metabolic transporter components.

The exchange of substances between circulating blood and brain can be determined by evaluating octanol/H₂0 partition coefficient, facilitated transport, and/or facilitated efflux. The methods of measuring blood brain barrier integrity can be used to identify suitable central nervous system modulators for use in the methods and compositions described herein.

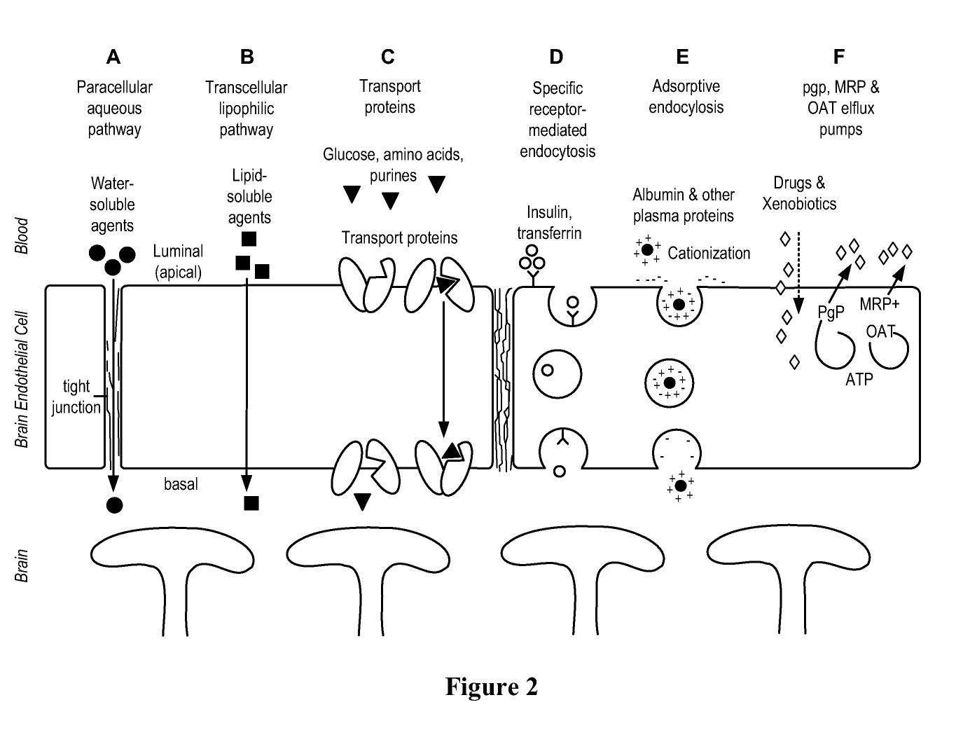

Various transporters exist to regulate rate of brain permeation for compounds with varying lipophilicity (see FIG. 2). Generally, hydrophilic nutrients, such as glucose and amino acids, are allowed entry into the physiological compartments of the methods and compositions disclosed herein. Conversely, compounds with low lipophilicity are pumped away from the physiological compartments by, for example, xenobiotic efflux transporters. These transporters are preferably modulated by the methods and compositions described herein to prevent entry of compounds and drugs into the central nervous system.

The blood CSF barrier is formed by the tight junctions of the epithelium of the choroid plexus and arachnoid membrane surrounding the brain and spinal cord. It is involved in micronutrient extraction, clearance of metabolic waste, and transport of drugs.

Mechanisms and routes of compounds into and out of brain include—paracellular aqueous pathway for water soluble agents, transcellular lipophilic pathway for lipid soluble agents, transport proteins for glucose, amino acids, purines, etc., specific receptor mediated endocytosis for insulin, transferrin, etc., adsorptive endocytosis for albumin, other plasma proteins, etc., and transporters (e.g., blood-brain barrier transport proteins) such as P-glycoprotein (P-gP), multi-drug resistance proteins (MRP), organic anion transporter (OAT) efflux pumps, gamma-aminobutyric acid (GABA) transporters and other transporters that modulate transport of drugs and other xenobiotics. Methods and compositions of the invention may involve modulation of one or more of these transporters. Preferably, the central nervous system modulators affect one or more of these mechanisms and routes to extrude drugs from the central nervous system.

The methods and compositions described herein also modulate other CNS barriers, such as neuronal transport barriers, as well as other CNS barriers.

In some embodiments, the blood brain barrier is modulated with a nitric oxide synthase (NOS) inhibitor. Preferably, the NOS inhibitor is a NOS-3 inhibitor. Non-limiting examples of NOS-3 inhibitors include analogs of L-arginine, such as N^{G}-Monomethyl-L-Arginine (L-NMMA), L-N-Methyl Arginine (L-NMA), N^{G}-Nitro-L-Arginine Methyl Ester (L-NAME), 7-nitroindazole (7-NI). See WO 00/23102, herein incorporated by reference in its entirety.

B. Blood-Tissue Barrier Transporters

In some embodiments, the invention provides methods and compositions that modulate ATP Binding Cassette (ABC) transport proteins. ABC transport proteins are a superfamily of membrane transporters with similar structural features. These transport proteins are widely distributed in prokaryotic and eukaryotic cells. They are critical in the maintenance of barrier to foreign molecules and removal of waste from privileged spaces, and may be overexpressed in certain glial tumors conferring drug resistance to cytotoxic drugs. 48 members of the superfamily are described. There are 7 major subfamilies, which include ABC A-G. Subfamilies C, B, and G play a role in transport activity at blood brain barrier and blood-CSF barrier. ABC A substrates include lipids and cholesterol; ABC B transporters include P-glycoprotein (P-gP) and other multi drug resistance proteins (MRPs); ABC C contains MRP proteins; ABC E is expressed in ovary, testis and spleen; and ABC G contains breast cancer resistance protein (BCRP).

Other examples of blood-CSF barrier transporters that can be modulated by methods and compositions of the invention include organic anion transport systems (OAT), P-gP, and the GABA transporters—GAT-1 and GAT2/BGT-1. Substrate compounds for OATs include opiate peptides, including enkephalin and deltorphin II, anionic compounds, indomethacin, salicylic acid and cimetidine. OATs are inhibited by baclofen, tagamet, indomethacin, etc. and transport HVA (dopamine metabolite) and metabolites of norepinephrine, epinephrine, 5-HT3, and histamine.

GABA transporters are Na and Cl dependent, and are specific for GABA, taurine, β alanine, betaine, and nipecotic acid. GAT2 transporters are localized to abluminal and luminal surfaces of capillary endothelial cells. GAT-1 is localized to the outside of neurons and glia. GABA-transporter substrates include lorazepam, midazolam, diazepam, klonazepam and baclofen. Probenicid inhibits luminal membrane GABA transporters from capillary endothelial cells. GAT-1 is inhibited by Tiagabine.

In some embodiments, the invention provides methods and compositions that modulate P-gP, e.g., that activate P-gP. P-gP, also known as ABCB1, forms a protective barrier to pump away by excreting compounds into bile, urine, and intestinal lumen. Three isoforms have been identified in rodents (mdr1a, mdr1b, mdr2) and two in humans (MDR1 and MDR2). It is expressed in epithelium of the brain choroid plexus (which forms the blood-cerebrospinal fluid barrier), as well as on the luminal surface of blood capillaries of the brain (blood-brain barrier) and other tissues known to have Blood-Tissue barriers, such as the placenta, the ovaries, and the testes.

In the brain, P-gP is expressed in multiple cell types within brain parenchyma including astrocytes and microglia and in luminal plasma membrane of capillary endothelium where it acts as a barrier to entry and efflux pump activity. P-gP transports a wide range of substrates out of cerebral endothelial cells into vascular lumen. P-gP is also expressed in the apical membrane of the choroid plexus and may transport substances into CSF.

P-gP substrates include molecules that tend to be lipophilic, planar molecules or uncharged or positively charged molecules. Non-limiting examples include organic cations, weak organic bases, organic anions and other uncharged compounds, including polypeptides and peptide derivatives, aldosterone, anthracyclines, colchicine, dexamethasone, digoxin, diltiazem, HIV protease inhibitors, loperamide, MTX, morphine, ondansetron, phenyloin and β-blockers. Inhibitors of P-gP include quinidine, verapamil, rifampin, PSC 833 (see Schinkel, J. Clin Invest., 1996, herein incorporated by reference in its entirety), carbamazepine, and anitryptiline.

Multi-drug resistance protein (MRP) substrates include acetaminophen glucuronide, protease inhibitors, methotrexate and ampicillin. Inhibitors of MRP include buthionine sulphoximine, an inhibitor of glutathione biosynthesis.

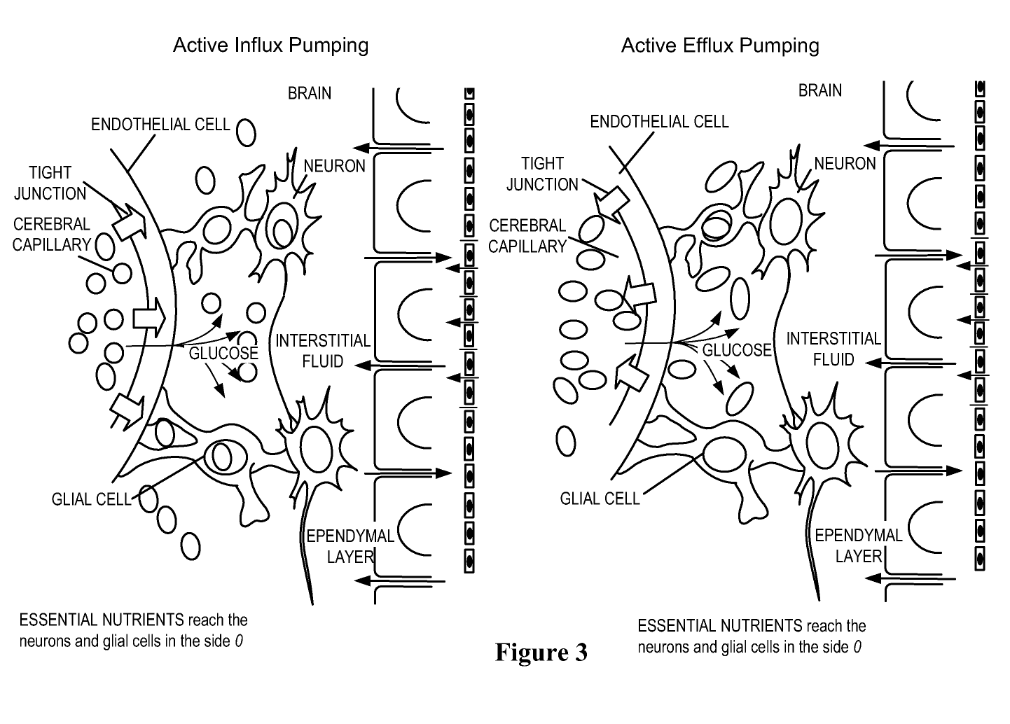

Further information on transporters that can be modulated in embodiments of the methods and compositions of the invention are provided in Table 1 below. FIG. 3 also provides an illustration of active transporters for both influx and efflux.

TABLE 1
Active Transporters in the Blood-Tissue Barrier

| | Physiological Function in Blood-Brain | |
|---|---|---|
| Active Transporter | Barrier | Exemplary Substrates |
| P-glycoprotein (P-gP) | Limits accumulation in CNS of phospholipids, | Loperamide, morphine, β endorphin, |
| | xenobiotics and other drugs; regulates | phenytoin, elavil, depakote, |
| | absorption, distribution and elimination of drug | cyclosporine, protease inhibitors, |
| | substances. | digoxin, calcium channel blockers, |
| | | vinca alkaloids, anthracyclines, |
| | | ivermectin, aldosterone, |
| | | hydrocortisone, dexamethasone, |
| | | taxanes, domperidone, ondansetron |
| Multidrug Resistance | MRP family members mediate ATP dependent | Acetaminophen glucuronide, protease |
| (MRP) Protein Family | transport of unconjugated, amphillic anions, and | inhibitors, methotrexate, ampicillin |
| | lipophillic compounds conjugated to | |
| | glutathione, glucuronic acid, and sulfate; | |
| | detoxification function includes extrusion of | |
| | leukotriene metabolites; folate transport. | |
| GABA transporters | GAT1 drives GABA into neurons; mediates | Lorazepam, midazolam, diazepam, |
| (GAT-1 and GAT-2, | clearance of GABA from the brain | klonazepam, baclofen |
| BGT-1) | | |
| Organic Anion Transport | Limits thiopurine uptake; transports HVA | Opiate peptides, including enkephalin |
| (OAT) Systems | (dopamine metabolite), and metabolites of | and deltorphin II, anionic compounds, |
| | norepinephrine, epinephrine, serotonin and | indomethacin, salicylic acid, cimetide |
| | histamine | |

C. Placental Barriers

Access to the fetus from the maternal circulation is controlled by the placenta, a physical barrier that separates the blood supply of the mother and fetus. The major function of the placenta is to transfer nutrients and oxygen from the mother to the fetus and to assist in the removal of waste products from the fetus to the mother. The placenta, therefore, provides a link between the maternal and fetal circulations while simultaneously acting as a barrier to protect the fetus from foreign substances in the maternal blood. Thus, some embodiments of the methods and compositions described herein are for the modulation of access of drugs, calcineurin inhibitors, chemicals and other substances through the placenta. In some embodiments, the methods and compositions involve the modification of the placental barrier to prevent the entry of drugs through the placental barrier and into the fetal environment, e.g., by efflux of drugs across the placenta.

Modulation of the placental barrier to prevent entry of drugs or other foreign substances to the fetal environment is important because of the sensitivity of the fetus to such substances. Studies have shown that nearly all drugs that are administered during pregnancy will enter, to some degree, the circulation of the fetus via passive diffusion, potentially harming the fetus during its growth and developmental stages. See, e.g., Syme, M. R. et al., Clin. Pharmacokinet. 43:487-514 (2004), herein incorporated by reference in its entirety. In addition, the fetus may be additionally harmed by drugs that are actively pumped across the placenta by various transporters located on both the fetal and maternal side of the trophoblast layer. Facilitated diffusion also appears to be a minor transfer mechanism for some drugs. Modulation of the entry pathways through the placenta, therefore, is important to preventing fetal exposure to drugs and other substances present in the maternal circulation.

### Placental Development and Anatomy

One of the functions of the placenta, in addition to its barrier-purpose, is to connect the fetus to the uterine wall near the fundus uteri, and more frequently on the posterior than on the anterior wall of the uterus. The placenta during fetal development is formed through the interweaving of both fetal and maternal portions, which allows the close proximity localization of the maternal and fetal circulation systems.

The fetal portion of the placenta consists of the villi of the chorion frondosum. These structures branch repeatedly, and increase in size throughout the fetal developmental stages. The chorion frondosum villi are suspended in the intervillous space where they are bathed in maternal blood. The circulation within the villi are conveyed to the space by the uterine arteries and carried away by the uterine veins. A branch of an umbilical artery enters each villus and ends in a capillary plexus from which the blood is drained by a tributary of the umbilical vein. The vessels of the villus are surrounded by a thin layer of mesoderm consisting of gelatinous connective tissue, which is covered by two strata of ectodermal cells derived from the trophoblast: the deeper stratum. The next layer of tissue consists of the mesodermic tissue, which represents the cytotrophoblast or layer of Langhans. The superficial layer, which is in contact with the maternal blood, is the syncytiotrophoblast. After the fifth month, the two strata of cells are replaced by a single layer of flattened cells.

The maternal portion of the placenta is formed by the decidua placentalis containing the intervillous space. As mentioned above, this space is produced by the enlargement and intercommunication of the spaces in the trophoblastic network. The changes involve the disappearance of the greater portion of the stratum compactum, but the deeper part of this layer persists and is condensed to form what is known as the basal plate. Between the basal plate and the uterine muscular fibers are the stratum spongiosum and the boundary layer. Through the stratum spongiosum, boundary layer and the basal plate, the uterine arteries and veins pass to and from the intervillous space. The endothelial lining of the uterine vessels ceases at the point where they terminate in the intervillous space, which is lined by the syncytiotrophoblast. Portions of the stratum compactum persist and are condensed to form a series of septa, which extend from the basal plate through the thickness of the placenta and subdivide it into the lobules or cotyledons seen on the uterine surface of the detached placenta. The cotyledons function as a vascular unit within the placenta.

The fetal and maternal blood currents traverse the placenta, the former passing through the blood vessels of the placental villi and the latter through the intervillous space. The two circulations do not intermingle, being separated from each other by the delicate walls of the villi. Nevertheless, the fetal blood is able to absorb, through the walls of the villi, oxygen and nutritive materials from the maternal blood, and give up to the latter its waste products. The purified blood is carried back to the fetus by the umbilical vein. The placenta, therefore, not only establishes a mechanical connection between the mother and the fetus, but also provides nutrition, respiration, and excretion services for the fetus.

During embryonic and early fetal development, the maternal blood does not communicate with the fetal circulation through the placenta. Maternal blood does not perfuse the placenta during the embryonic period and the feto-placental-maternal circulation does not become established until around the tenth week of pregnancy. Hence, access of drugs and other chemicals present in the maternal blood during the first 10 weeks of gestation occurs via diffusion through extracellular fluid. Maternal blood access to the placental circulation only occurs after development and establishment of the feto-placental-maternal circulation.

D. Placental Transport Mechanisms

Transplacental exchanges are known to involve passive transfer, active transport, facilitated diffusion, phagocytosis and pinocytosis. See, e.g., Pacifici G M, et al., Clin. Pharmacokinet. 28:235-69 (1995), herein incorporated by reference. Studies, however, have shown that phagocytotic and pinocytotic mechanisms are too slow to have any significant influence on drug or chemical transfer from the maternal circulation to the fetus. Syme et al. (2004). Therefore, one embodiment of the methods and compositions disclosed herein is to modulate passive transfer, facilitated diffusion and active transport of drugs, calcineurin inhibitors, chemicals and other substances across the placental barrier.

Passive Transfer

One embodiment is the modulation of passive transfer of drugs, chemicals and other substances across the placental barrier. Passive transfer represents the permeation of a molecule through a physical barrier, such as a cell membrane, down its concentration gradient. Passive diffusion does not require the input of energy, is not saturable and is not subject to competitive inhibition. When drugs cross the placenta by passive diffusion, the amount that crosses in any given time is dependent on the concentration of the drug in the maternal circulation, its physicochemical properties and the properties of the placenta that determine how readily the drug will pass.

Passive diffusion is favored for low-molecular weight and highly lipid-soluble drugs that are predominantly un-ionized. The placenta resembles a lipid bilayer membrane, so only the non-protein bound portion of a drug, barring any applicable active-transport mechanisms, is free to diffuse across it.

Facilitated Diffusion

Another embodiment of the methods and compositions disclosed herein is the modulation of facilitated diffusion mechanisms in the placental barrier. Facilitated diffusion requires the presence of a carrier substance within the placenta. Moreover, the transport of the system becomes saturated at high concentrations relative to the Michaelis-Menten constant (Kₘ) of the transporter. However, transport by this mechanism does not require the input of energy, as opposed to active transport of substances. Facilitated diffusion usually equalizes the concentration of drugs, chemicals, or substances between the maternal and fetal circulations. It may be that for many substances, such as carbohydrates, facilitated diffusion provides a means to increase transport rates when the functional and metabolic needs of the fetus would not be met by passive diffusion alone. Folkart G R, et al. Am. J. Obstet. Gynecol., 80:221-223 (1960), herein incorporated by reference.

Studies have shown that only a few drugs use facilitated diffusion mechanisms to traverse the placental barrier. Ganciclovir has been demonstrated to be taken up into maternal-facing syncytiotrophoblast vesicles by a carrier-dependent system. Henderson G I et al., Am. J. Med. Sci. 306:151-156 (1993). However, transport of Ganciclovir probably involves a combination of passive and facilitated diffusion mechanisms, the rate-limiting transfer step being passive diffusion. Syme et al. (2004). Placental carrier-mediated transport systems have also been found in maternal-facing syncytiotrophoblast membrane vesicles for cephalosporin, cephalexin and glucocorticoids. Kudo Y, et al., Biochim. Biophys. Acta 731:415-420 (1989); Fant M E, et al., Biochim. Biophys. Acta 731:415-420 (1983), incorporated by reference herein. In light of the relatively few drugs that use this mechanism, it has been suggested that structurally related endogenous compounds, such as hormones and nucleosides, will most likely be the primary species to benefit from this transport system. Syme et al. (2004).

Active Transporters

Another embodiment of the methods and compositions disclosed herein is use of modulators or calcineurin inhibitors in manipulating active transport of drugs, chemicals and other substances across the placental barrier. Active transport across the placental barrier, as opposed to facilitated diffusion or passive transport, requires energy, usually in the form of adenosine triphosphate (ATP) or through energy stored in the transmembrane electrochemical gradient provided by Na⁺, Cl⁻ or H⁺. Because of the input of energy, active transport systems may work against a concentration gradient, however, saturation of the transporters can occur.

Extensive studies have been conducted regarding placental transport systems of nutrients, such as amino acids, vitamins and glucose. See Hahn T, et al., Early Pregnancy 2:168-182 (1996); Moe A J, Arm. J. Physiol. 268:C1321-1331 (1995); Bissonnette J M, Mead Johnson Symp. Perinat. Dev. Med., 18:21-23 (1981), all incorporated herein by reference. Active transport of drugs occurs through the same transport systems, most likely due to structurally similarities between the transported drugs and endogenous substrates. Syme et al. (2004).

Active drug transporters are located either in the maternal-facing brush border (apical) membrane or the fetal-facing basolateral (basal) membrane where they pump drugs into or out of the synctiotrophoblast. Table 2 summarizes the active transporters that have been identified in the placenta.

TABLE 2
Active transporters in Placenta

| Active Transporter | Physiological Function in Placenta | Exemplary Substrates |
|---|---|---|
| P-glycoprotein (P-gP) | Fetal-to-maternal transfer of hydrophobic | Digoxin, cyclosporine, saquinavir, |
| | cationic compounds | vincristine, vinblastine, paclitaxel, |
| | | dexamethasone, terfenadine, |
| | | sirolimus, quinidine, ondansetron, |
| | | loperamide |
| Multidrug resistance | Fetal-to-maternal transfer of glutathione, | Methotrexate, etoposide, |
| protein 1 (MRP1) | sulfate and glucuronide conjugates | vincristine, cisplatin, vinblastine, |
| | (dianionic sulfated bile salts) | HIV protease inhibitors |
| Multidrug resistance | Fetal-to-maternal transfer of glutathione, | Etoposide, cisplatin, doxorubicin, |
| protein 2 (MRP2) | sulfate and glucuronide conjugates | vincristine, vinblastine, |
| | (dianionic sulfated bile salts, bilirubin | methotrexate, paracetamol, |
| | glucoronide, estradiol glucuronide) | glucuronide, grepafloxacin, |
| | | ampilicillin |
| Multidrug resistance | Fetal-to-maternal transfer of anionic | Methotrexate, etoposide |
| protein 3 (MRP3) | conjugates | |
| Breast cancer resistant | Unknown | Topotecan, mitoxantrone, |
| protein (BCRP) | | doxorubicin, daunorubicin |
| Serotonin transporter | Serotonin transfer | Amphetamines |
| (SERT) | | |
| Norepinephrine transporter | Dopamine and norepinephrine transfer | Amphetamines |
| (NET) | | |
| Extraneuronal monoamine | Serotonin, dopamine, norepinephrine, | Amphetamines, imipramine, |
| transporter (OCT3) | histamine transfer | desipramine, clonidine, cimetidine |
| Organic cation transporters | Maternal-to-fetal transfer of carnitine | Metamphetamine, quinidine, |
| (OCTN) | | verapamil, pyrilamine |
| Monocarboxylate | Fetal-to-maternal transfer of lactate and | Valproic acid |
| transporters | pyruvate | |
| Dicarboxylate transporters | Maternal-to-fetal transfer of succinate and α- | Unknown |
| | ketoglutarate | |
| Sodium/multivitamin | Maternal-to-fetal transfer of biotin and | Carbamazepine, primidone |
| transporter (SMVT) | pantothenate | |

P-Glycoproteins (P-gP)

Another embodiment of the methods and compositions disclosed herein is the modulation of the placental P-gP transporter. The multidrug resistant gene (MDR1) product, P-glycoprotein, is a member of the ATP-binding cassette (ABC) transporter family. In the placenta, P-gP is expressed in the trophoblast cells of the brush-border membrane, but not the basal membrane. Cordon-Cardo C. et al., J. Histochem. Cytochem. 38:1277-87 (1990); Sugawara I, et al., Cancer Res. 48:1926-1929 (1988), herein incorporated by reference in its entirety. Studies have demonstrated that placental P-gP regulates the transfer of cyclosporine, vincristine, vinblastine and digoxin into trophoblast cells. Ushigome F, et al., Eur. J. Pharmacol. 408:1-10 (2000); Pavek P, et al., J. Pharm. Sci. 10:1583-1592 (2001), herein incorporated by reference. However, the transfers of the drugs were predominantly in the fetal-to-maternal transfer direction, thereby reducing fetal exposure to the drugs. Ushigame et al. (2000).

Studies in the mdr1a (P-gP) knockout (−/−) mouse demonstrate the importance of the P-gP transporter in reducing fetal exposure to drugs and other chemicals or substances. For example, Lankas et al. (Reprod. Toxicol. 12:457-463 (1998), herein incorporated by reference) has shown that administration of an isomer of the pesticide avermectin was associated with a 100% incidence of fetal cleft palate in the mdr1a knockout mice. In contrast, heterozygous (+/−) mice were less sensitive and homozygous (+/+) mice insensitive at the same doses tested on the knockout mice. In addition, the degree of chemical exposure was inversely related to the expression of P-gP, which was determined by fetal genotyping. Other studies in mdr1a knockout mice have confirmed the major fetoprotective role that the P-gP transporter plays. Smit J W, et al., J. Clin. Invest. 104:1441-1447 (1999).

Multidrug Resistance Associated Protein (MRP) Family

Another embodiment of the methods and compositions disclosed herein is the modulation of placental MRP transporters. The MRP family consists of seven members, designated MRP1 to MRP7. For review, see Borst P, et al., J. Natl. Cancer Inst. 92:1295-1302 (2000), herein incorporated by reference. In human placenta, at least three members of the MRP family have been identified: MRP1, MRP2 and MRP3. Sugawara I, et al., Cancer Lett. 112:23-31 (1997); St-Pierre V, et al., Am. J. Physiol. Regul. Integr. Comp. Physiol. 279:R1495-1503 (2000); Flens M J et al., Am. J. Pathol. 148:1237-1247 (1996), herein incorporated by reference. MRP 1 and MRP 3 were found to be localized primarily in the fetal endothelial cells of the placenta microcapillary. Hipfner D R, et al., Biochim. Biophys. Acta 1461:359-376 (1999). MRP2, MRP3, and to a lesser extent MRP1, are also expressed in the apical membrane of the synctiotrophoblast. Sugawara et al. (1997); Flens et al. (1996) and St.-Pierre et al. (2000).

MRP-related placental proteins transport a variety of substrates primarily in the direction of the fetal-to-maternal transfer. Accordingly, researchers have suggested that MRP-transporters could exert a feto-protective role by the removal of metabolic end products from the fetus to the mother. St.-Pierre et al. (2000); Cui Y, et al., Mol. Pharmacol. 55:929-937 (1999), herein incorporated by reference.

Breast Cancer Resistant Protein (BCRP)

Another embodiment of the methods and compositions disclosed herein is the modulation of placental BCRP transporters. BCRP, an ATP-driven transporter, is highly expressed in the placenta. Allikrnets R., et al., Cancer Res. 58:5337-5339 (1998), herein incorporated by reference. BCRP is responsible for rendering tumor cells resistant to chemocalcineurin inhibitors, such as topotecan, mitoxantrone, doxorubicin and daunorubicin. Allen J D, et al., Cancer Res. 59:4237-4241 (1999). BCRP has also been shown to restrict the passage of topotecan and mitoxantrone to the fetus in mice. Jonker J W et al., J. Natl. Cancer Inst. 92:1651-1656 (2000), herein incorporated by reference.

Monoamine Transporters

Yet another embodiment is the modulation of monoamine transporters in placenta. Studies have identified the placental monoamine transporters as serotonin transporter (SERT), norepinephrine transporter (NET) and the extraneuronal monoamine transporter (OCT3). Ramamoorthy S, et al., Placenta 14:449-461 (1993); Ramamoorthy S., et al., Biochem. 32:1346-1353 (1993); Kekuda R., et al., J. Biol. Chem. 273:15971-15979 (1998), all herein incorporated by reference. SERT and NET derive energy from the transmembrane Na⁺ and Cl⁻ electrochemical gradient, and are primarily localized in the brush-border membrane of the placental trophoblast. Both SERT and NET transport serotonin, dopamine and norepinephrine from the maternal circulation to the fetus. Drug substrates of the SERT and NET transporters include amphetamines, although cocaine and non-tricyclic antidepressants bind to the SERT and NET transporters with high affinity without being transferred across the membrane.

OCT3 is localized to the basal membrane, where it transports serotonin, dopamine, norepinephrine and histamine via a Na⁺ and Cl⁻ independent system. Ganaphthy V et al., J. Pharmacol. Exp. Ther. 294:413-420 (2000); Kekuda et al. (1998). Amphetamines, imipramine and desipramine may be actively transported by placental OCT3.

Organic Cation Transporters

One additional embodiment of the present invention is the modulation of placental Organic Cation Transporters. Placental Na+-driven organic cation transporter 2 (OCTN2) has been identified and localized to the basal membrane of the synctiotrophoblast. Wu X et al., J. Pharmacol. Exp. Ther. 290:1482-1492 (1999), herein incorporated by reference. Placental OCTN2 transports carnitine across the placenta in the direction of the maternal-to-fetal transfer. Ohashi R., et al., J. Pharmacol. Exp. Ther. 291:778-784 (1999), herein incorporated by reference. Studies have identified metamphetamine, quinidine, verapamil, pyrilamine, desipramine, dimethylamiloride, cimetidine, and procainimide as drug substrates for OCTN2. Wu X, et al., Biochem. Biophys. Res. Commun. 246:589-595 (1998); Wu X, et al., Biochim. Biophys. Acta 1466:315-327 (2000), herein incorporated by reference.

Monocarboxylate Transporters and the Dicarboxylate Transporters

Another embodiment of the methods and compositions disclosed herein is the modulation of monocarboxylate (MCT) and dicarboxylate (NaDC3 transporters. Both MCT (e.g. lactate transport) and NaDC3 (e.g. succinate transport), which utilize electrochemical gradients for transport, are localized to the brush border membrane of the placenta, with MCT being expressed in the basal membrane to a lesser extent. Price N T, et al., Biochem. J. 329:321-328 (1998); Ganaphthy V, et al., Biochem J. 249:179-184 (1988); Balkovetz D F, et al., 263:13823-13830 (1988), all incorporated by reference herein. Valproic acid, a teratogenic substance, may be a substrate for MCT transfer, and compete with lactate for transport across the placental barrier. Nakamura H. et al., Pharm. Res. 19:154-161 (2002), herein incorporated by reference.

Transporter Modulators (e.g., Activators or Inhibitors) The invention provides compositions and methods for reducing or eliminating the effects of a calcineurin inhibitor in the CNS and/or in the fetus. In some embodiments, the compositions and embodiments described herein modulate the efflux of calcineurin inhibitor out of physiological compartments, including across the blood brain barrier barrier via a BTB or fetal transport protein, e.g., the P-gP transporter. In some embodiments, such modulators activate and/or increase the efflux by the BTB or fetal transport protein, e.g., P-gP transporters on the blood brain barrier barriers.

Modulators may be any suitable modulator. In some embodiments, modulators useful in the invention are polyphenols, such as flavonoids. Suitable modulators include catechins from green tea, including (−) epicatechin. See Wang, E, et al., Biochem. Biophys. Res. Comm. 297:412-418 (2002); Zhou, S., et al., Drug Metabol. Rev. 36:57-104 (2004), both of which are herein incorporated by reference in its entirety. Other suitable modulators, e.g., P-gP modulators for use herein include flavonols, including, but not limited to, kaempferol, quercetin, and galangin.

In other embodiments, P-gP transporter modulators may include small molecules, including 2-p-Tolyl-5,6,7,8-tetrahydrobenzo[d]imidazo[2,1-b]thiazole; 1-Carbazol-9-yl-3-(3,5-dimethylpyrazol-1-yl)-propan-2-ol; 2-(4-Chloro-3,5-dimethylphenoxy)-N-(2-phenyl-2H-benzotriazol-5-yl)-acetamide; N-[2-(4-Chloro-phenyl)-acetyl]-N′-(4,7-dimethyl-quinazolin-2-yl)-guanidine; 1-Benzyl-7,8-dimethoxy-3-phenyl-3H-pyrazolo[3,4-c]isoquinoline; N-(3-Benzooxazol-2-yl-4-hydroxyphenyl)-2-p-tolyloxyacetamide; 8-Allyl-2-phenyl-8H-1,3a,8-triazacyclopenta[a]indene; 3-(4-Chloro-benzyl)-5-(2-methoxyphenyl)-[1,2,4]oxadiazole; 2-Phenethylsulfanyl-5,6,7,8-tetrahydrobenzo[4,5]thieno[2,3-d]pyrimidin-4-ylamine; (5,12,13-Triaza-indeno[1,2-b]anthracen-13-yl)-acetic acid ethyl ester; 2,2′-(1-phenyl-¹H-1,2,4-triazole-3,5-diyl)bis-phenol; and 2-(2-Chloro-phenyl)-5-(5-methylthiophen-2-yl)-[1,3,4]oxadiazole. See Kondratov, et al., Proc. Natl. Acad. Sci. 98:14078-14083 (2001), herein incorporated by reference in its entirety.

In one embodiment, a P-gP substrate is used to inhibit transport across the blood brain barrier and/or the placenta. Multi Drug Resistance Proteins consist of a family of plasma membrane proteins encoded by the MDR (multidrug resistance) gene.

In some embodiments, the invention utilizes a modulator of a BTB transport protein. In some embodiments, the invention utilizes a modulator of a BTB transport protein that is an ABC transport protein. In some embodiments, the invention utilizes a BTB transport protein activator. In some embodiments, the BTB transport protein modulator is a modulator of P-gP, e.g., an activator of P-gP.

One class of compounds useful in the compositions and methods of the invention is polyphenols. Many polyphenols are modulators of BTB transport proteins; however, any suitable polyphenol that produces a decrease of one or more CNS effects of a calcineurin inhibitor, no matter what the mechanism, may be used in the compositions and methods of the invention.

A particularly useful class of polyphenols is the flavonoids. Flavonoids, the most abundant polyphenols in the diet, can be classified into subgroups based on differences in their chemical structures. The basic flavonoid structure is shown below (formula I):

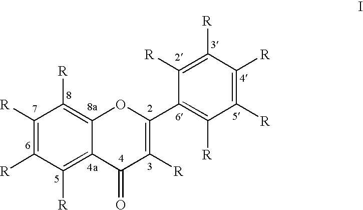

wherein the 2,3 bond may be saturated or unsaturated, and wherein each R can be independently selected from the group consisting of hydrogen, substituted or unsubstituted hydroxyl, substituted or unsubstituted amine, substituted or unsubstituted thiol, substituted or unsubstituted C₁-C₁₀ alkyl, substituted or unsubstituted C₁-C₁₀ alkynyl, substituted or unsubstituted C₁-C₁₀ alkenyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether, carbohydrate, and substituted carbohydrate; and its pharmaceutically acceptable salts, esters, prodrugs, analogs, isomers, stereoisomers or tautomers thereof.

“Carbohydrate” as used herein, includes, but not limited to, monosaccharides, disaccharides, oligosaccharides, or polysaccharides. Monosaccharide for example includes, but not limited to, allose, altrose, mannose, gulose, Idose, glucose, galactose, talose, and fructose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, α-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, inulin, chitin, amylose and amylopectin.

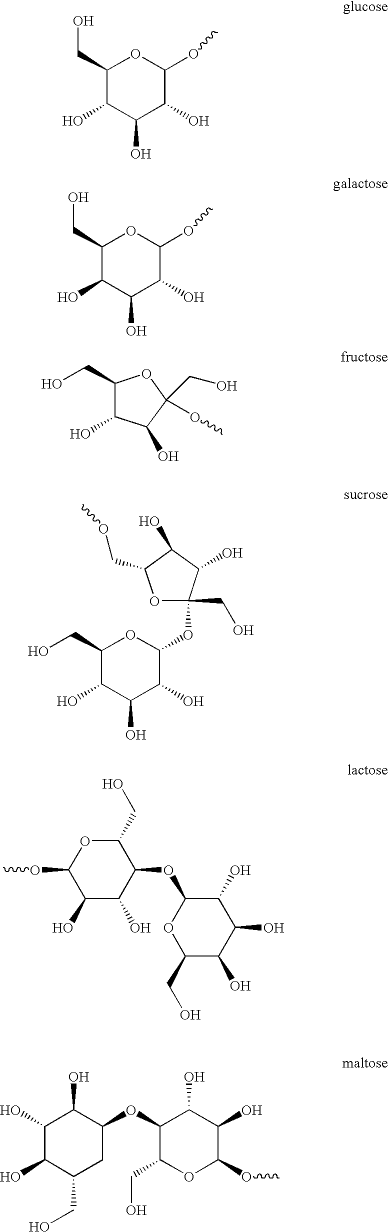

In some embodiments, the invention utilizes a flavonoid where the molecule is planar. In some embodiments, the invention utilizes a flavonoid where the 2-3 bond is unsaturated. In some embodiments, the invention utilizes a flavonoid where the 3-position is hydroxylated. In some embodiments, the invention utilizes a flavonoid where the 2-3 bond is unsaturated and the 3-position is hydroxylated (e.g., flavonols).

In some embodiments, the invention utilizes one or more flavonoids selected from the group consisting of quercetin, isoquercetin, flavone, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments, the invention utilizes one or more flavonoids selected from the group consisting of quercetin, isoquercetin, apigenin, rhoifolin, galangin, fisetin, morin, rutin, kaempferol, myricetin, naringenin, hesperetin, phloretin, and genistein. Structures of these compounds are well-known in the art. See, e.g., Critchfield et al. (1994) Biochem. Pharmacol 7:1437-1445.

In some embodiments, the invention utilizes a flavonol. In some embodiments, the flavonol is selected from the group consisting of quercetin, fisetin, morin, rutin, myricetin, galangin, and kaempherol, and combinations thereof. In some embodiments, the flavonol is selected from the group consisting of quercetin, galangin, and kaempherol, and combinations thereof. In some embodiments, the flavonol is quercetin or a substituted analog thereof. In some embodiments, the flavonol is galangin. In some embodiments, the flavonol is kaempherol.

A particularly useful flavonol is quercetin. Quercetin may be used to illustrate formulations and methods useful in the invention, however, it is understood that the discussion of quercetin applies equally to other flavonoids, flavonols, and polyphenols useful in the invention, e.g., kaempferol and galangin.

The structure of quercetin is shown below (formula II):

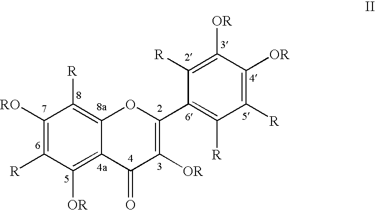

wherein each OR is an OH (i.e., 3-OH, 5-OH, 7-OH, 3′-OH, and 4′-OH) and each R is an H. The numbering of the carbons is the same as in Formula I. This form of quercetin is used in some embodiments of the invention. As used herein, the term “quercetin” also encompasses derivatives of quercetin, wherein each R can be independently selected from the group consisting of hydrogen, substituted or unsubstituted C₁-C₁₀ alkyl, substituted or unsubstituted aryl, substituted or unsubstituted C₁-C₁₀ aliphatic acyl, substituted or unsubstituted C₁-C₁₀ aromatic acyl, trialkyl silyl, substituted or unsubstituted ether, carbohydrate, and substituted carbohydrate; and its pharmaceutically acceptable salts, esters, prodrugs, analogs, isomers, stereoisomers or tautomers thereof. In addition, metabolites of quercetin, e.g., quercetin 3-O-glucouronide, are encompassed by the term “quercetin” as used herein.

In some embodiments, the quercetin is in a carbohydrate-derivatized form, e.g., a quercetin-O-saccharide. Quercetin-O-saccharides useful in the invention include, but are not limited to, quercetin 3-O-glycoside, quercetin 3-O-glucorhamnoside, quercetin 3-O-galactoside, quercetin 3-O-xyloside, and quercetin 3-O-rhamnoside. In some embodiments, the invention utilizes a quercetin 7-O-saccharide.

In some embodiments, the invention utilizes a quercetin aglycone. In some embodiments, a combination of aglycones and carbohydrate-derivatized quercetins is used. It will be appreciated that the various forms of quercetin may have different properties useful in the compositions and methods of the invention, and that the route of administration can determine the choice of forms, or combinations of forms, used in the composition or method, Choice of a single form, or of combinations, is a matter of routine experimentation.

Thus, in some embodiments the invention features a composition or method utilizing quercetin to reduce or eliminate one or more side or fetal effects of a calcineurin inhibitor, such as tacrolimus or a tacrolimus analog.

In some embodiments, the quercetin is provided in a form for oral consumption. Oral bioavailability of quercetin O-saccharides is generally superior to that of quercetin aglycones. The bioavailability of the various components is dependent on 1) the site of carbohydrate moiety or moieties and ii) the pendant sugar unit. In addition it is believed that specific carriers are responsible for the absorption of various quercetin glycosides, as well as specific intestinal betaglucosidases. After distribution in the body, the major metabolite, quercetin glucuronide (e.g., quercetin 3-O-glucouronid), is found. Oral bioavailability is sensitive to the presence of food factors.

In compositions for oral delivery of quercetin, carbohydrate-derivatized forms (also referred to herein as “quercetin saccharides”) are used in some embodiments. In some embodiments, quercetin-3-O-glycoside is used in an oral preparation of quercetin; in some embodiments, a pharmaceutically acceptable excipient is included in the composition. In some embodiments, quercetin 3-O-glucorhamnoside is used in an oral preparation of quercetin; in some embodiments, a pharmaceutically acceptable excipient is included in the composition. In some embodiments, a combination of quercetin-3-O-glycoside and quercetin 3-O-glucorhamnoside is used in an oral preparation of quercetin; in some embodiments, a pharmaceutically acceptable excipient is included in the composition. Other carbohydrate-derivatized forms of quercetin, or other forms of quercetin which are derivatives as described above, can also be used, based on their oral bioavailability, their metabolism, their incidence of gastrointestinal or other side effects, and other factors known in the art. Determining the bioavailability of quercetin in the form of derivatives including aglycones and glycosides is a matter of routine experimentation. See, e.g., Graefe et al., J. Clin. Pharmacol. (2001) 451:492-499; Arts et al.(2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

In some embodiments, the invention provides a composition for administration of quercetin to an animal, e.g., for the oral delivery of quercetin to reduce a side effect of a calcineurin inhibitor, that contain at least about 1, 5, 10, 20, 30, 40, 50, 60, 70, 80, 90, 95, 99, 99.5, 99.9, or 99.99% quercetin-O-saccharide. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-O-saccharide. In some embodiments, the invention provides a composition that contains about 1-100% quercetin-O-saccharide, or about 10-100% quercetin-O-saccharide, or about 20-100% quercetin-O-saccharide, or about 50-100% quercetin-O-saccharide, or about 80-100% quercetin-O-saccharide, or about 90-100% quercetin-O-saccharide, or about 95-100% quercetin-O-saccharide, or about 99-100% quercetin-O-saccharide. In some embodiments, the invention provides a composition that contains about 1-90% quercetin-O-saccharide, or about 10-90% quercetin-O-saccharide, or about 20-90% quercetin-O-saccharide, or about 50-90% quercetin-O-saccharide, or about 80-90% quercetin-O-saccharide. In some embodiments, the invention provides a composition that contains about 1-75% quercetin-O-saccharide, or about 10-75% quercetin-O-saccharide, or about 20-75% quercetin-O-saccharide, or about 50-75% quercetin-O-saccharide. In some embodiments, the invention provides a composition that contains about 1-50% quercetin-O-saccharide, or about 10-50% quercetin-O-saccharide, or about 20-50% quercetin-O-saccharide, or about 30-50% quercetin-O-saccharide, or about 40-50% quercetin-O-saccharide. In some embodiments, the invention provides a composition that contains about 1-40% quercetin-O-saccharide, or about 10-40% quercetin-O-saccharide, or about 20-40% quercetin-O-saccharide, or about 30-40% quercetin-O-saccharide. In some embodiments, the invention provides a composition that contains about 1-30% quercetin-O-saccharide, or about 10-30% quercetin-O-saccharide, or about 20-30% quercetin-O-saccharide. In some embodiments, the invention provides a composition that contains about 1-20% quercetin-O-saccharide, or about 10-20% quercetin-O-saccharide. In some embodiments, the invention provides a composition that contains about 1-10% quercetin-O-saccharide. In some embodiments, the invention provides a composition that contains about 1, 2, 5, 10, 20, 30, 40, 50, 60, 70, 80, 90, 95, 96, 97, 98, or 99% quercetin-O-saccharide.

In some embodiments, the invention provides a composition for administration of quercetin to an animal, e.g., for the oral delivery of quercetin to reduce a side effect of a calcineurin inhibitor, that contain at least about 1, 5, 10, 20, 30, 40, 50, 60, 70, 80, 90, 95, 99, 99.5, 99.9, or 99.99% quercetin-3-O-glycoside. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-3-O-glycoside. In some embodiments, the invention provides a composition that contains about 1-100% quercetin-3-O-glycoside, or about 10-100% quercetin-3-O-glycoside, or about 20-100% quercetin-3-O-glycoside, or about 50-100% quercetin-3-O-glycoside, or about 80-100% quercetin-3-O-glycoside, or about 90-100% quercetin-3-O-glycoside, or about 95-100% quercetin-3-O-glycoside, or about 99-100% quercetin-3-O-glycoside. In some embodiments, the invention provides a composition that contains about 1-90% quercetin-3-O-glycoside, or about 10-90% quercetin-3-O-glycoside, or about 20-90% quercetin-3-O-glycoside, or about 50-90% quercetin-3-O-glycoside, or about 80-90% quercetin-3-O-glycoside. In some embodiments, the invention provides a composition that contains about 1-75% quercetin-3-O-glycoside, or about 10-75% quercetin-3-O-glycoside, or about 20-75% quercetin-3-O-glycoside, or about 50-75% quercetin-3-O-glycoside.

In some embodiments, the invention provides a composition that contains about 1-50% quercetin-3-O-glycoside, or about 10-50% quercetin-3-O-glycoside, or about 20-50% quercetin-3-O-glycoside, or about 30-50% quercetin-3-O-glycoside, or about 40-50% quercetin-3-O-glycoside. In some embodiments, the invention provides a composition that contains about 1-40% quercetin-3-O-glycoside, or about 10-40% quercetin-3-O-glycoside, or about 20-40% quercetin-3-O-glycoside, or about 30-40% quercetin-3-O-glycoside. In some embodiments, the invention provides a composition that contains about 1-30% quercetin-3-O-glycoside, or about 10-30% quercetin-3-O-glycoside, or about 20-30% quercetin-3-O-glycoside. In some embodiments, the invention provides a composition that contains about 1-20% quercetin-3-O-glycoside, or about 10-20% quercetin-3-O-glycoside. In some embodiments, the invention provides a composition that contains about 1-10% quercetin-3-O-glycoside. In some embodiments, the invention provides a composition that contains about 1, 2, 5, 10, 20, 30, 40, 50, 60, 70, 80, 90, 95, 96, 97, 98, or 99% quercetin-3-O-glycoside.

In some embodiments, the invention provides a composition for administration of quercetin to an animal, e.g., for the oral delivery of quercetin to reduce a side effect of a calcineurin inhibitor, that contain at least about 1, 5, 10, 20, 30, 40, 50, 60, 70, 80, 90, 95, 99, 99.5, 99.9, or 99.99% quercetin-3-O-glucorhamnoside. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-3-O-glucorhamnoside. In some embodiments, the invention provides a composition that contains about 1-100% quercetin-3-O-glucorhamnoside, or about 10-100% quercetin-3-O-glucorhamnoside, or about 20-100% quercetin-3-O-glucorhamnoside, or about 50-100% quercetin-3-O-glucorhamnoside, or about 80-100% quercetin-3-O-glucorhamnoside, or about 90-100% quercetin-3-O-glucorhamnoside, or about 95-100% quercetin-3-O-glucorhamnoside, or about 99-100% quercetin-3-O-glucorhamnoside. In some embodiments, the invention provides a composition that contains about 1-90% quercetin-3-O-glucorhamnoside, or about 10-90% quercetin-3-O-glucorhamnoside, or about 20-90% quercetin-3-O-glucorhamnoside, or about 50-90% quercetin-3-O-glucorhamnoside, or about 80-90% quercetin-3-O-glucorhamnoside. In some embodiments, the invention provides a composition that contains about 1-75% quercetin-3-O-glucorhamnoside, or about 10-75% quercetin-3-O-glucorhamnoside, or about 20-75% quercetin-3-O-glucorhamnoside, or about 50-75% quercetin-3-O-glucorhamnoside. In some embodiments, the invention provides a composition that contains about 1-50% quercetin-3-O-glucorhamnoside, or about 10-50% quercetin-3-O-glucorharnnoside, or about 20-50% quercetin-3-O-glucorhamnoside, or about 30-50% quercetin-3-O-glucorhamnoside, or about 40-50% quercetin-3-O-glucorhamnoside. In some embodiments, the invention provides a composition that contains about 1-40% quercetin-3-O-glucorhamnoside, or about 10-40% quercetin-3-O-glucorhamnoside, or about 20-40% quercetin-3-O-glucorhamnoside, or about 30-40% quercetin-3-O-glucorhamnoside. In some embodiments, the invention provides a composition that contains about 1-30% quercetin-3-O-glucorhamnoside, or about 10-30% quercetin-3-O-glucorhamnoside, or about 20-30% quercetin-3-O-glucorhamnoside. In some embodiments, the invention provides a composition that contains about 1-20% quercetin-3-O-glucorhamnoside, or about 10-20% quercetin-3-O-glucorhamnoside. In some embodiments, the invention provides a composition that contains about 1-10% quercetin-3-O-glucorhamnoside. In some embodiments, the invention provides a composition that contains about 1, 2, 5, 10, 20, 30, 40, 50, 60, 70, 80, 90, 95, 96, 97, 98, or 99% quercetin-3-O— glucorhamnoside.

In some embodiments, the invention provides a composition for administration of quercetin to an animal, e.g., for the oral delivery of quercetin to reduce a side effect of a calcineurin inhibitor, that contain at least about 1, 5, 10, 20, 30, 40, 50, 60, 70, 80, 90, 95, 99, 99.5, 99.9, or 99.99% quercetin aglycone. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin aglycone. In some embodiments, the invention provides a composition that contains about 1-100% quercetin aglycone, or about 10-100% quercetin aglycone, or about 20-100% quercetin aglycone, or about 50-100% quercetin aglycone, or about 80-100% quercetin aglycone, or about 90-100% quercetin aglycone, or about 95-100% quercetin aglycone, or about 99-100% quercetin aglycone. In some embodiments, the invention provides a composition that contains about 1-90% quercetin aglycone, or about 10-90% quercetin aglycone, or about 20-90% quercetin aglycone, or about 50-90% quercetin aglycone, or about 80-90% quercetin aglycone. In some embodiments, the invention provides a composition that contains about 1-75% quercetin aglycone, or about 10-75% quercetin aglycone, or about 20-75% quercetin aglycone, or about 50-75% quercetin aglycone. In some embodiments, the invention provides a composition that contains about 1-50% quercetin aglycone, or about 10-50% quercetin aglycone, or about 20-50% quercetin aglycone, or about 30-50% quercetin aglycone, or about 40-50% quercetin aglycone. In some embodiments, the invention provides a composition that contains about 1-40% quercetin aglycone, or about 10-40% quercetin aglycone, or about 20-40% quercetin aglycone, or about 30-40% quercetin aglycone. In some embodiments, the invention provides a composition that contains about 1-30% quercetin aglycone, or about 10-30% quercetin aglycone, or about 20-30% quercetin aglycone. In some embodiments, the invention provides a composition that contains about 1-20% quercetin aglycone, or about 10-20% quercetin aglycone. In some embodiments, the invention provides a composition that contains about 1-10% quercetin aglycone. In some embodiments, the invention provides a composition that contains about 1, 2, 5, 10, 20, 30, 40, 50, 60, 70, 80, 90, 95, 96, 97, 98, or 99% quercetin aglycone.

In some embodiments, the invention provides a composition for administration of quercetin to an animal, e.g., for the oral delivery of quercetin to reduce a side effect of a calcineurin inhibitor, that contains a combination of quercetin-O-saccharides. In some embodiments, the invention provides a composition for administration of quercetin to an animal to reduce a side effect of a calcineurin inhibitor, e.g., for the oral delivery of quercetin, that contain a combination of quercetin-3-O-glycoside and quercetin-3-O-glucorhamnoside. In these compositions, the ranges or amounts of the quercetin-O-saccharides, e.g., quercetin-3-O-glycoside and quercetin-3-O-glucorhamnoside may be any suitable combination of the ranges or amounts, above.

In some embodiments, the invention provides a composition for administration of quercetin to an animal, e.g., for the oral delivery of quercetin to reduce a side effect of a calcineurin inhibitor, that contains a combination of one or more quercetin-O-saccharides and quercetin aglycone In some embodiments, the invention provides a composition for administration of quercetin to an animal to reduce a side effect of a calcineurin inhibitor, e.g., for the oral delivery of quercetin, that contain a combination of quercetin-3-O-glycoside and quercetin aglycone. In these compositions, the ranges or amounts of quercetin-3-O-glycoside and quercetin aglycone may be any suitable combination of the ranges or amounts, above. In some embodiments, the invention provides a composition for administration of quercetin to an animal to reduce a side effect of a calcineurin inhibitor, e.g., for the oral delivery of quercetin, that contain a combination of quercetin-3-O— glucorhamnoside and quercetin aglycone. In these compositions, the ranges or amounts of quercetin-3-O— glucorhamnoside and quercetin aglycone may be any suitable combination of the ranges or amounts, above. In some embodiments, the invention provides a composition for administration of quercetin to an animal to reduce a side effect of a calcineurin inhibitor, e.g., for the oral delivery of quercetin, that contain a combination of quercetin-3-O-glycoside, quercetin-3-O— glucorhamnoside and quercetin aglycone. In these compositions, the ranges or amounts of quercetin-3-O-glycoside, quercetin-3-O-glucorhamnoside and quercetin aglycone may be any suitable combination of the ranges or amounts, above. Other quercetin saccharides, as described herein and as known in the art or developed, may be used as well.

In some embodiments, Quercetin may be modified to increase its solubility by derivatizing with at least one phosphate group. The phosphate group can be attached to any suitable part of the quercetin molecule. In some embodiments, Quercetin may be modified to increase its solubility by attaching an amino acid such as glycine, alanine, dimethyl glycine, sarcosine, aspartic acid, or arginine. The amino acid can be attached to any suitable part of the quercetin molecule.

In some of these embodiments, a pharmaceutically acceptable excipient is also included.

Calcineurin Inhibitors

The invention provides compositions and methods, e.g., to reduce or eliminate side effects of a calcineurin inhibitor. In some embodiments, the invention provides compositions and methods to reduce or eliminate side effects of a calcineurin inhibitor in the CNS and/or fetus. In some embodiments, the compositions and methods retain or enhance a desired effect of the calcineurin inhibitor, e.g., a peripheral effect. In some embodiments, the compositions and methods change the concentration of a calcineurin inhibitor in a physiological compartment. The methods and compositions of the invention apply to any calcineurin inhibitor for which it is desired to reduce one or more side effects, e.g., CNS and/or fetal effects. In some embodiments, the compositions and methods of the invention utilize CsA. In some embodiments, the compositions and methods of the invention utilize tacrolimus. In some embodiments, the calcineurin inhibitor is tacrolimus analog. In some embodiments, the tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

### Tacrolimus

Tacrolimus, also known as FK506, is the active ingredient in Prograf, one of the leading market immunosuppressants from preventing transplant rejection. Tacrolimus is a macrolide immunosuppressant that can be produced by Streptomyces tsukubaensis. It chemical name is [3S-[3R[E(1S*,3S*,4S*)], 4S*,5R*,8S*,9E,12R*,14R*,15S*,16R*,18S*,19S*,26aR*]]-,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21 (4H,23H)-tetrone, monohydrate. The chemical structure of tacrolimus is:

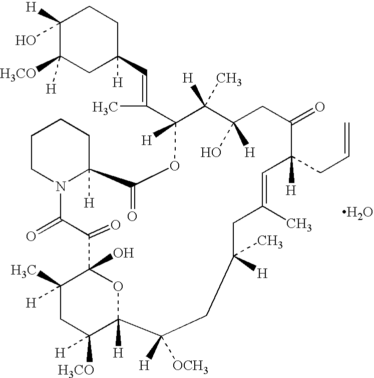

Its empirical formula is C₄₄H₆₉NO₁₂.H₂O (formula weight of 822.03). Early studies demonstrated the immunosuppressive properties of tacrolimus in vitro. At subnanomolar concentrations, tacrolimus was shown to inhibit the proliferation of murine or human T cells stimulated by specific antigens, antibodies to the T cell receptor (TCR)/CD3 complex or mitogenic lectins as well as the generation of cytolytic T cells (CTL) in mixed lymphocyte reactions. In these initial experiments, it became clear that tacrolimus exerts its activity by disrupting calcium signaling events that lead to lymphokine production, similar to cyclosporin A (CsA), but with 50-100-fold higher potency. Animal models of transplantation confirmed the immunosuppressive properties of tacrolimus and its higher potency over CsA. However, these animal studies also revealed that tacrolimus had major side effects, including neurotoxicity and nephrotoxicity, much like CsA. Despite these toxicity results, trials with tacrolimus were initiated as rescue therapy in human liver transplant patients who did not fare well with CsA treatment. In many of these patients, tacrolimus proved quite beneficial and had a lifesaving effect. These findings were further substantiated upon extended use of the drug in a larger group of patients, providing the impetus for controlled, multi-center clinical trials of tacrolimus as a primary therapy in liver and kidney transplantation. It was demonstrated that tacrolimus-based therapy offers a number of potential advantages over conventional CsA-based treatment, such as a corticosteroid-sparing action and a significant reduction in incidence of both acute and corticosteroid—resistant rejection episodes. Tacrolimus was approved by the FDA for the prophylaxis of liver transplant rejection in 1994 and kidney transplant rejection in 1997.

Tacrolimus prolongs the survival of the host and transplanted graft in animal transplant models of liver, kidney, heart, bone marrow, small bowel and pancreas, lung and trachea, skin, cornea, and limb. In animals, tacrolimus has been demonstrated to suppress some humoral immunity and, to a greater extent, cell-mediated reactions such as allograft rejection, delayed type hypersensitivity, collagen-induced arthritis, experimental allergic encephalomyelitis, and graft versus host disease.

Without being limited to any theory, tacrolimus inhibits T lymphocyte activation, although the exact mechanism is unknown, experimental data suggest that upon formation of a complex with the intracellular protein, FK506-binding protein 12 (FKBP12), the drug selectively inhibits the enzymatic activity of the calcium/calmodulin-dependent protein phosphatase, calcineurin. Engagement of the T cell receptor (TCR) initiates at least two separate signaling pathways driven by Ras/PKC and an elevation of intracellular Ca²⁺. The latter activates calcineurin, composed of a catalytic subunit, a regulatory subunit and of calmodulin. Enzymatically active calcineurin can dephosphorylate the cytoplasmic NFAT family members and cause the dissociation of the inhibitor IkB from NFkB. NFAT and NFkB are then translocated into the nucleus where they can interact with their DNA binding sequences on the IL-2 promoter. To be transcriptionally active, NFAT needs to form a complex with accessory factors, such as AP-1 (fos/jun) contributed by the Ras/PKC pathway. Calcineurin is also thought to regulate the activity of Oct-1 through induction of its co-activators OAP and BOB-1. The complex formed between FKBP12 and tacrolimus impedes access of calcineurin to its substrates and thereby, prevents the nuclear translocation or activation of these factors. These factors are thought to initiate gene transcription for the formation of lymphokines (such as interleukin-2, gamma interferon). Calcineurin may also affect the function of the c-jun N-terminal kinase, JNK and of Elk-1, which are components of Ras/PKC driven signaling mechanisms. The net result is that T lymphocyte activation is inhibited resulting in immunosuppression.

The greatest limitation to the therapeutic potential of tacrolimus comes from its toxic side effects, which include neurotoxicity, nephrotoxicity, diabetogenicity, and gastrointestinal disturbances. The precise pathophysiological mechanisms of tacrolimus toxicity are still enigmatic, in part because the cells that are actually implicated within the target tissues of this toxicity have not been clearly identified. However, evidence has accumulated that the side effects of tacrolimus arise from the same biochemical mechanisms that underlie its immunosuppressive effects, namely an inhibition of calcineurin activity in various tissues. This is suggested by the fact that the toxicity profile of tacrolimus overlaps with that of CsA and is totally different from that of rapamycin, an immunosuppressant that also binds FKBP12 but unlike tacrolimus it does not inhibit calcineurin. Furthermore, FKBP12-binding analogs of tacrolimus that do not inhibit calcineurin function are devoid of toxicity and the antagonist of FK506-induced immunosuppression, L-685,818, can block FK506-induced toxicity in animal models.

Neurotoxicity, including tremor, headache, and other changes in motor function, mental status, and sensory function were reported in approximately 55% of liver transplant recipients in the two randomized studies. Tremor occurred more often in Prograf-treated kidney transplant patients (54%) and heart transplant patients (15%) compared to cyclosporine-treated patients. The incidence of other neurological events in kidney transplant and heart transplant patients was similar in the two treatment groups. Tremor and headache have been associated with high whole-blood concentrations of tacrolimus and may respond to dosage adjustment. Seizures have occurred in adult and pediatric patients receiving Prograf. Coma and delirium also have been associated with high plasma concentrations of tacrolimus.

### Side Effect

Liver Transplantation

The principal adverse reactions of Prograf are tremor, headache, diarrhea, hypertension, nausea, and abnormal renal function. These occur with oral and IV administration of Prograf and may respond to a reduction in dosing. Diarrhea was sometimes associated with other gastrointestinal complaints such as nausea and vomiting. Hyperkalemia and hypomagnesemia have occurred in patients receiving Prograf therapy. Hyperglycemia has been noted in many patients; some may require insulin therapy.

The incidence of adverse events was determined in two randomized comparative liver transplant trials among 514 patients receiving tacrolimus and steroids and 515 patients receiving a cyclosporine-based regimen (CBIR). The proportion of patients reporting more than one adverse event was 99.8% in the tacrolimus group and 99.6% in the CBIR group. Precautions must be taken when comparing the incidence of adverse events in the U.S. study to that in the European study. The 12-month post-transplant information from the U.S. study and from the European study is presented below. The two studies also included different patient populations and patients were treated with immunosuppressive regimens of differing intensities. Adverse events reported in >15% in tacrolimus patients (combined study results) are presented below for the two controlled trials in liver transplantation:

LIVER TRANSPLANTATION: ADVERSE EVENTS OCCURRING IN ≧15% OF
PROGRAF-TREATED PATIENTS

| | U.S. STUDY | | EUROPEAN STUDY | |
|---|---|---|---|---|
| | Prograf | CBIR | Prograf | |
| | (N = 250) | (N = 250) | (N = 264) | CBIR (N = 265) |
| Nervous System | | | | |
| Headache (see WARNINGS) | 64% | 60% | 37% | 26% |
| Tremor (see WARNINGS) | 56% | 46% | 48% | 32% |
| Insomnia | 64% | 68% | 32% | 23% |
| Paresthesia | 40% | 30% | 17% | 17% |
| Gastrointestinal | | | | |
| Diarrhea | 72% | 47% | 37% | 27% |
| Nausea | 46% | 37% | 32% | 27% |
| Constipation | 24% | 27% | 23% | 21% |
| LFT Abnormal | 36% | 30% | 6% | 5% |
| Anorexia | 34% | 24% | 7% | 5% |
| Vomiting | 27% | 15% | 14% | 11% |
| Cardiovascular | | | | |
| Hypertension (see | 47% | 56% | 38% | 43% |
| PRECAUTIONS) | | | | |
| Urogenital | | | | |
| Kidney Function Abnormal (see | 40% | 27% | 36% | 23% |
| WARNINGS) | | | | |
| Creatinine Increased (see | 39% | 25% | 24% | 19% |
| WARNINGS) | | | | |
| BUN Increased (see | 30% | 22% | 12% | 9% |
| WARNINGS) | | | | |
| Urinary Tract Infection | 16% | 18% | 21% | 19% |
| Oliguria | 18% | 15% | 19% | 12% |
| Metabolic and Nutritional | | | | |
| Hyperkalemia (see WARNINGS) | 45% | 26% | 13% | 9% |
| Hypokalemia | 29% | 34% | 13% | 16% |
| Hyperglycemia (see WARNINGS) | 47% | 38% | 33% | 22% |
| Hypomagnesemia | 48% | 45% | 16% | 9% |
| Hemic and Lymphatic | | | | |
| Anemia | 47% | 38% | 5% | 1% |
| Leukocytosis | 32% | 26% | 8% | 8% |
| Thrombocytopenia | 24% | 20% | 14% | 19% |
| Miscellaneous | | | | |
| Abdominal Pain | 59% | 54% | 29% | 22% |
| Pain | 63% | 57% | 24% | 22% |
| Fever | 48% | 56% | 19% | 22% |
| Asthenia | 52% | 48% | 11% | 7% |
| Back Pain | 30% | 29% | 17% | 17% |
| Ascites | 27% | 22% | 7% | 8% |
| Peripheral Edema | 26% | 26% | 12% | 14% |
| Respiratory System | | | | |
| Pleural Effusion | 30% | 32% | 36% | 35% |
| Atelectasis | 28% | 30% | 5% | 4% |
| Dyspnea | 29% | 23% | 5% | 4% |
| Skin and Appendages | | | | |
| Pruritus | 36% | 20% | 15% | 7% |
| Rash | 24% | 19% | 10% | 4% |

Kidney Transplantation

The most common adverse reactions reported were infection, tremor, hypertension, abnormal renal function, constipation, diarrhea, headache, abdominal pain and insomnia. Adverse events that occurred in >5% of Prograf-treated kidney transplant patients are presented below:

KIDNEY TRANSPLANTATION: ADVERSE EVENTS OCCURRING
IN ≧15% OF PROGRAF-TREATED PATIENTS

| | Prograf | CBIR |
|---|---|---|
| | (N = 205) | (N = 207) |
| Nervous System | | |
| Tremor (see WARNINGS) | 54% | 34% |
| Headache (see WARNINGS) | 44% | 38% |
| Insomnia | 32% | 30% |
| Paresthesia | 23% | 16% |
| Dizziness | 19% | 16% |
| Gastrointestinal | | |
| Diarrhea | 44% | 41% |
| Nausea | 38% | 36% |
| Constipation | 35% | 43% |
| Vomiting | 29% | 23% |
| Dyspepsia | 28% | 20% |
| Cardiovascular | | |
| Hypertension (see PRECAUTIONS) | 50% | 52% |
| Chest pain | 19% | 13% |
| Urogenital | | |
| Creatinine Increased (see | 45% | 42% |
| WARNINGS) | | |
| Urinary Tract Infection | 34% | 35% |
| Metabolic and Nutritional | | |
| Hypophosphatemia | 49% | 53% |
| Hypomagnesemia | 34% | 17% |
| Hyperlipemia | 31% | 38% |
| Hyperkalemia (see WARNINGS) | 31% | 32% |
| Diabetes Mellitus (see WARNINGS) | 24% | 9% |
| Hypokalemia | 22% | 25% |
| Hyperglycemia (see WARNINGS) | 22% | 16% |
| Edema | 18% | 19% |
| Hemic and Lymphatic | | |
| Anemia | 30% | 24% |
| Leukopenia | 15% | 17% |
| Miscellaneous | | |
| Infection | 45% | 49% |
| Peripheral Edema | 36% | 48% |
| Asthenia | 34% | 30% |
| Abdominal Pain | 33% | 31% |
| Pain | 32% | 30% |
| Fever | 29% | 29% |
| Back Pain | 24% | 20% |
| Respiratory System | | |
| Dyspnea | 22% | 18% |
| Cough Increased | 18% | 15% |
| Musculoskeletal | | |
| Arthralgia | 25% | 24% |
| Skin | | |
| Rash | 17% | 12% |
| Pruritus | 15% | 7% |

Heart Transplantation

The more common adverse reactions in Prograf-treated heart transplant recipients were abnormal renal function, hypertension, diabetes mellitus, CMV infection, tremor, hyperglycemia, leukopenia, infection, and hyperlipemia. Adverse events in heart transplant patients in the European trial are presented below:

HEART TRANSPLANTATION: ADVERSE EVENTS OCCURRING
IN ≧15% OF PROGRAF-TREATED PATIENTS

| | Prograf + | |
|---|---|---|
| COSTART Body System | Azathioprine | CsA + Azathioprine |
| COSTART Term | (n = 157) | (n = 157) |
| Cardiovascular System | | |
| Hypertension (See | 62% | 69% |
| PRECAUTIONS) | | |
| Pericardial effusion | 15% | 14% |
| Body as a Whole | | |
| CMV infection | 32% | 30% |
| Infection | 24% | 21% |
| Metabolic and Nutritional Disorders | | |
| Hyperlipemia | 18% | 27% |
| Diabetes Mellitus (See | 26% | 16% |
| WARNINGS) | | |
| Hyperglycemia (See | 23% | 17% |
| WARNINGS) | | |
| Hemic and Lymphatic System | | |
| Leukopenia | 48% | 39% |
| Anemia | 50% | 36% |
| Urogenital System | | |
| Kidney function abnormal (See | 56% | 57% |
| WARNINGS) | | |
| Urinary tract infection | 16% | 12% |
| Respiratory System | | |
| Bronchitis | 17% | 18% |
| Nervous System | | |
| Tremor (See WARNINGS) | 15% | 6% |

In the European study, the cyclosporine trough concentrations were above the pre-defined target range (i.e., 100-200 ng/mL) at Day 122 and beyond in 32-68% of the patients in the cyclosporine treatment arm, whereas the tacrolimus trough concentrations were within the pre-defined target range (i.e., 5-15 ng/mL) in 74-86% of the patients in the tacrolimus treatment arm. Only selected targeted treatment-emergent adverse events were collected in the US heart transplantation study. Those events that were reported at a rate of 15% or greater in patients treated with Prograf and mycophenolate mofetil include the following: any target adverse events (99.1%), hypertension (88.8%), hyperglycemia requiring antihyperglycemic therapy (70.1%), hypertriglyceridemia (65.4%), anemia (hemoglobin <10.0 g/dL) (65.4%), fasting blood glucose >140 mg/dL (on two separate occasions) (60.7%), hypercholesterolemia (57.0%), hyperlipidemia (33.6%), WBCs <3000 cells/mcL (33.6%), serious bacterial infections (29.9%), magnesium <1.2 mEq/L (24.3%), platelet count <75,000 cells/mcL (18.7%), and other opportunistic infections (15.0%). Other targeted treatment-emergent adverse events in Prograf-treated patients occurred at a rate of less than 15%, and include the following: Cushingoid features, impaired wound healing, hyperkalemia, Candida infection, and CMV infection/syndrome.

The following adverse effects were also reported in liver, kidney, and/or heart transplant recipients who were treated with tacrolimus in clinical trials (Astellas package insert, April 2006):

Nervous System

Abnormal dreams, agitation, amnesia, anxiety, confusion, convulsion, crying, depression, dizziness, elevated mood, emotional lability, encephalopathy, haemorrhagic stroke, hallucinations, headache, hypertonia, incoordination, insomnia, monoparesis, myoclonus, nerve compression, nervousness, neuralgia, neuropathy, paresthesia, paralysis flaccid, psychomotor skills impaired, psychosis, quadriparesis, somnolence, thinking abnormal, vertigo, writing impaired, abnormal vision, amblyopia, ear pain, otitis media, and tinnitus.

Special Senses

Abnormal vision, amblyopia, ear pain, otitis media, tinnitus.

Gastrointestinal

Anorexia, cholangitis, cholestatic jaundice, diarrhea, duodenitis, dyspepsia, dysphagia, esophagitis, flatulence, gastritis, gastroesophagitis, gastrointestinal hemorrhage, GGT increase, GI disorder, GI perforation, hepatitis, hepatitis granulomatous, ileus, increased appetite, jaundice, liver damage, liver function test abnormal, nausea, nausea and vomiting, oesophagitis ulcerative, oral moniliasis, pancreatic pseudocyst, rectal disorder, stomatitis, vomiting.

Cardiovascular

Abnormal ECG, angina pectoris, arrhythmia, atrial fibrillation, atrial flutter, bradycardia, cardiac fibrillation, cardiopulmonary failure, cardiovascular disorder, chest pain, congestive heart failure, deep thrombophlebitis, echocardiogram abnormal, electrocardiogram QRS complex abnormal, electrocardiogram ST segment abnormal, heart failure, heart rate decreased, hemorrhage, hypotension, peripheral vascular disorder, phlebitis, postural hypotension, syncope, tachycardia, thrombosis, vasodilatation.

Urogenital

Acute kidney failure, albuminuria, bladder spasm, cystitis, dysuria, hematuria, hydronephrosis, kidney failure, kidney tubular necrosis, nocturia, oliguria, pyuria, toxic nephtropathy, urge incontinence, urinary frequency, urinary incontinence, urinary retention, vaginitis.

Metabolic/Nutritional

Acidosis, alkaline phosphatase increased, alkalosis, ALT (SGPT) increased, AST (SGOT) increased, bicarbonate decreased, bilirubinemia, BUN increased, dehydration, edema, GGT increased, gout, healing abnormal, hypercalcemia, hypercholesterolemia, hyperkalemia, hyperlipemia, hyperphosphatemia, hyperuricemia, hypervolemia, hypocalcemia, hypoglycemia, hypokalemia, hypomagnesemia, hyponatremia, hypophosphatemia, hypoproteinemia, lactic dehydrogenase increase, peripheral edema, weight gain.

Endocrine

Cushing's syndrome, diabetes mellitus

Hemic/Lymphatic

Coagulation disorder, ecchymosis, haematocrit increased, haemoglobin abnormal, hypochromic anemia, leukocytosis, leukopenia, polycythemia, prothrombin decreased, serum iron decreased, thrombocytopenia.

Miscellaneous

Abdomen enlarged, abdominal pain, abscess, accidental injury, allergic reaction, asthenia, back pain, cellulitis, chills, fall, feeling abnormal, fever, flu syndrome, generalized edema, hernia, mobility decreased, pain, peritonitis, photosensitivity reaction, sepsis, temperature intolerance, ulcer.

Musculoskeletal

Arthralgia, cramps, generalized spasm, joint disorder, leg cramps, myalgia, myasthenia, osteoporosis

Respiratory

Asthma, bronchitis, cough increased, dyspnea, emphysema, hiccups, lung disorder, lung function decreased, pharyngitis, pleural effusion, pneumonia, pneumothorax, pulmonary edema, respiratory disorder, rhinitis, sinusitis, voice alteration.

Skin

Acne, alopecia, exfoliative dermatitis, fungal dermatitis, herpes simplex, herpes zoster, hirsutism, neoplasm skin benign, skin discoloration, skin disorder, skin ulcer, sweating.

Post Marketing Adverse Events

The following adverse events have been reported from worldwide marketing experience with Prograf. There have been rare spontaneous reports of myocardial hypertrophy associated with clinically manifested ventricular dysfunction in patients receiving Prograf therapy.

Other events include:

Cardiovascular

Atrial fibrillation, atrial flutter, cardiac arrhythmia, cardiac arrest, electrocardiogram T wave abnormal, flushing, myocardial infarction, myocardial ischaemia, pericardial effusion, QT prolongation, Torsade de Pointes, venous thrombosis deep limb, ventricular extrasystoles, ventricular fibrillation.

Gastrointestinal

Bile duct stenosis, colitis, enterocolitis, gastroenteritis, gastrooesophageal reflux disease, hepatic cytolysis, hepatic necrosis, hepatotoxicity, impaired gastric emptying, liver fatty, mouth ulceration, pancreatitis haemorrhagic, pancreatitis necrotizing, stomach ulcer, venoocclusive liver disease.

Hemic/Lymphatic

Disseminated intravascular coagulation, neutropenia, pancytopenia, thrombocytopenic purpura, thrombotic thrombocytopenic purpura.

Metabolic/Nutritional

Glycosuria, increased amylase including pancreatitis, weight decreased.

Miscellaneous

Feeling hot and cold, feeling jittery, hot flushes, multi-organ failure, primary graft dysfunction.

Nervous System

Carpal tunnel syndrome, cerebral infarction, hemiparesis, leukoencephalopathy, mental disorder, mutism, quadriplegia, speech disorder, syncope.

Respiratory

Acute respiratory distress syndrome, lung infiltration, respiratory distress, respiratory failure Skin

Stevens-Johnson syndrome, toxic epidermal necrolysis.

Special Senses

Blindness, blindness cortical, hearing loss including deafness, photophobia.

Urogenital

Acute renal failure, cystitis haemorrhagic, hemolytic-uremic syndrome, micturition disorder.

Overdosage

Limited overdosage experience is available. Acute overdosages of up to 30 times the intended dose have been reported. Almost all cases have been asymptomatic and all patients recovered with no sequelae. Occasionally, acute overdosage has been followed by side effects consistent with those listed above except in one case where transient urticaria and lethargy were observed. Based on the poor aqueous solubility and extensive erythrocyte and plasma protein binding, it is anticipated that tacrolimus is not dialyzable to any significant extent; there is no experience with charcoal hemoperfusion. The oral use of activated charcoal has been reported in treating acute overdoses, but experience has not been sufficient to warrant recommending its use. Usually, general supportive measures and treatment of specific symptoms are followed in all cases of overdosage.

In acute oral and IV toxicity studies, mortalities were seen at or above the following doses: in adult rats, 52× the recommended human oral dose; in immature rats, 16× the recommended oral dose; and in adult rats, 16× the recommended human IV dose (all based on body surface area corrections).

The following adverse effects have been reported in patients treated with PROTOPIC Ointment (Tacrolimus for dermatologic use): headache, hyperesthesia, insomnia, depression, paresthesia, abnormal vision, anxiety, dizziness, seizures, syncope, tachycardia, migraine, photosensitivity reaction, thinking abnormal and vertigo.

The exact mechanism of neurotoxicity associated with tacrolimus in not known, although it might be attributed to the high accumulation of the drug in the brain. Even though tacrolimus has a high lipophilic property, its transport through the BBB into the brain is restricted, and this phenomenon is known to occur because of the P-glycoprotein (P-gP) encoded by the multidrug resistant I gene, ABCB1. The gene acts as an adenosine triphosphate-dependent membrane efflux pump to prevent the accumulation of various drugs in the brain. Tacrolimus is a substrate of P-gP and is pumped out from the brain (Kochi et al. Eur J Pharmacol 1999, 372: 287; Yokogawa et al. Pharm Res 1999, 16: 1213). This is supported by the finding that the concentration of tacrolimus in the brain was markedly increased by depleting the mdr1 gene in mice (Yokogawa et al. Pharm Res 1999, 16: 1213). It has been suggested that tacrolimus induced neurotoxicity may occur as a result of the reduced P-gP function. This report was based on the correlation of neurotoxic events of tacrolimus and ABCB1 polymorphisms associated with expressions levels and function of P-gP (Yamauchi et al. Transplantation 2002, 74 (4): 571-578).

The invention provides compositions and methods utilizing an agent that reduces or eliminates a side effect associated with calcineurin inhibitor treatment. The invention also provides compositions and methods utilizing an agent that increases a therapeutic effect associated with calcineurin inhibitor treatment. The invention also provides compositions and methods utilizing an agent that changes the concentration in a physiological compartment of a calcineurin inhibitor.

In some embodiments, the invention provides compositions and methods utilizing a combination of a calcineurin inhibitor and an agent that reduces or eliminates a side effect associated with calcineurin inhibitor treatment. Typically, the side effect-decreasing agent is a modulator of a blood brain barrier (BBB). It will be appreciated that BTB is also present in other cells in the body, and the invention provides methods and compositions to modulate BTB throughout the body of a subject. The invention also provides compositions and methods utilizing an agent that reduces or eliminates fetal effect associated with calcineurin inhibitor treatment.

The terms “BTB transport protein modulator” and “BTB transport protein modulator” are used interchangeably herein. The methods and compositions are useful in the treatment of an animal in need of treatment, where it is desired that one or more effects of the calcineurin inhibitor or a developing fetus be reduced or eliminated. In embodiments further utilizing a calcineurin inhibitor, the methods and compositions are useful in the treatment of an animal in need of treatment, where it is desired that one or more effects of calcineurin inhibitor or the developing fetus be reduced or eliminated while one or more of the therapeutic effects (e.g., peripheral effects) of the calcineurin inhibitor are retained or enhanced. In some embodiments, the methods and compositions of the invention utilize an agent that changes the concentration of a calcineurin inhibitor in a physiological compartment.

In some embodiments of the invention, the calcineurin inhibitor is tacrolimus or a tacrolimus analog. Examples of tacrolimus analogs include, but are not limited to, meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP 1903; rapamycin; dexamethasone-FK506 heterodimer; 13-O-demethyl tacrolimus; and FK 506-dextran conjugate.

The agent causing a decrease in the side effects of the calcineurin inhibitor, and/or an increase in a therapeutic effect of a calcineurin inhibitor, and/or a change in concentration of the calcineurin inhibitor in a physiological compartment, e.g., a modulator of a BTB transport protein may be an activator or an inhibitor of the protein. The modulatory effect may be dose-dependent, e.g., some modulators act as activators in one dosage range and inhibitors in another. In some embodiments, a modulator of a BTB transport protein is used in a dosage wherein it acts primarily as an activator.

Typically, the use of the BTB or placental barrier transport protein modulator, e.g., activator, results in a decrease in one or more side and/or fetal effects of the calcineurin inhibitor. The therapeutic effect(s) of the calcineurin inhibitor may be decreased, remain the same, or increase; however, in preferred embodiments, if the therapeutic effect is decreased, it is not decreased to the same degree as the side or fetal effects. It will be appreciated that a given calcineurin inhibitor may have more than one therapeutic effect and or one or more side or fetal effects, and it is possible that the therapeutic ratio (in this case, the ratio of change in desired effect to change in undesired effect) may vary depending on which effect is measured. However, at least one therapeutic effect of the calcineurin inhibitor is decreased to a lesser degree than at least one side effect of the calcineurin inhibitor. In some embodiments, the use of the BTB transport protein modulator does not affect the therapeutic effect(s) of the calcineurin inhibitor.

In addition, in some embodiments, one or more therapeutic effects of the calcineurin inhibitor is enhanced by use in combination with a BTB transport protein modulator, while one or more side effects of the calcineurin inhibitor is reduced or substantially eliminated. For example, in some embodiments, the immunosuppressant effect of the calcineurin inhibitor is enhanced while one or more side effects of the calcineurin inhibitor is reduced or substantially eliminated.

In some embodiments, the concentration of the calcineurin inhibitor is changed in a physiological compartment by using the calcineurin inhibitor in combination with a BTB transport protein modulator. Examples of physiological compartments include, but are not limited to, blood, liver, lymph node, spleen, peyer's patches, intestines, lungs, heart, kidney, pancreas, and gull bladder.

Without being bound by theory, and as an example only of a possible mechanism, it is thought that the methods and compositions of the invention operate by reducing or eliminating the concentration of the calcineurin inhibitor from the compartment where the side effect is produced (e.g., brain) and/or fetal compartment, while retaining or even increasing the effective concentration of the calcineurin inhibitor in the periphery and/or compartment where the therapeutic effect is desired. Calcineurin inhibitor act at least in part by peripheral mechanisms (e.g. inhibition of T lymphocyte activation) and may thus retain some or all of their activity, or even display enhanced therapeutic activity, while at the same time side and/or fetal effects are reduced or eliminated.

In some embodiments, the BTB transport protein modulator decreases the clearance of the calcineurin inhibitor from the compartment where the calcineurin inhibitor is exerting its therapeutic effect. Without being limited to any theory, and as an example only of a possible mechanism, it is thought that the methods and compositions of the invention operate by reducing or eliminating the concentration of the calcineurin inhibitor from the compartment where the calcineurin inhibitor is cleared from the animal (e.g., liver), hence, retaining or even increasing the effective concentration of the calcineurin inhibitor in the periphery and/or compartment where the therapeutic effect is desired.

It will be appreciated that the therapeutic and/or side effects of an calcineurin inhibitor may be mediated in part or in whole by one or more metabolites of the calcineurin inhibitor, and that a BTB transport modulator that reduces or eliminates the concentration of the calcineurin inhibitor and/or of one or active metabolites of the calcineurin inhibitor that produce side effects, while retaining or enhancing the concentration of the calcineurin inhibitor and/or one or more metabolites in the periphery and/or compartment that produces a therapeutic effect, is also encompassed by the methods and compositions of the invention. In addition, a BTB transport modulator itself may be metabolized to metabolites that have differing activities in the modulation of one or more BTB transport modulators, and these metabolites are also encompassed by the compositions and methods of the invention.

Hence, in some embodiments the invention provides compositions that include a calcineurin inhibitor and a Blood-Tissue barrier (BTB) transport modulator where the calcineurin inhibitor is present in an amount sufficient to exert a therapeutic effect and the BTB transport modulator is present in an amount sufficient to decrease a side effect of the calcineurin inhibitor when compared to the side effect without the BTB transport modulator when the composition is administered to an animal. The decrease in the side effect can be measurable. In some embodiments the invention provides compositions that include a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to change the concentration of the calcineurin inhibitor in a physiological compartment when compared to concentration of the calcineurin inhibitor in the physiological compartment without the BTB transport protein modulator, when the composition is administered to an animal. In some embodiments, the BTB transport protein modulator increases the concentration of a calcineurin inhibitor in a physiological compartment where a therapeutic effect is desired (e.g. periphery and/or T cells). In some embodiments, the BTB transport protein modulator decreases the concentration of a calcineurin inhibitor in a physiological compartment where a side effect is produced (e.g. brain). The change in concentration of the calcineurin inhibitor modulator in a physiological compartment can be measurable. The BTB transport protein modulator is a BTB activator in some embodiments. In some embodiments the BTB transport protein modulator is a modulator of ATP binding cassette (ABC) transport proteins. In some embodiments the BTB transport protein modulator is a modulator of P-glycoprotein (P-gP).

In some embodiments, compositions of the invention include one or more calcineurin inhibitor with one or more calcineurin inhibitor as well as one or more than one BTB transport protein modulator. One or more of the calcineurin inhibitors may have one or more side effects which are desired to be decreased.

It will be appreciated that when a BTB transport protein that is the target of the BTB transport modulator is present on the cells where the calcineurin inhibitor is exerting its therapeutic effect, the dosage of the BTB transport modulator may be adjusted such that the side effect of the calcineurin inhibitor are reduced without a substantial reduction of the therapeutic effect in the target cells. In some embodiments, it is desirable to inhibit a BTB transport protein present in the cells where the calcineurin inhibitor is exerting its therapeutic effect while activating the same or another BTB transport protein at other site(s) such that the side effect of the calcineurin inhibitor are reduced. Therefore, the dosage of the BTB transport modulator may be adjusted such that a BTB transport protein that is the target of the BTB transport modulator is inhibited on the cells where the calcineurin inhibitor is exerting its therapeutic effect, while the same or another BTB transport protein is activated on other site(s) to reduced the side effect of the calcineurin inhibitor.

Compositions of the invention may be prepared in any suitable form for administration to an animal. In some embodiments, the invention provides pharmaceutical compositions.

In some embodiments, the invention provides compositions suitable for oral administration. In some embodiments, compositions are suitable for transdermal administration. In some embodiments, compositions are suitable for injection by any standard route of injection, e.g., intravenous, subcutaneous, intramuscular, or intraperitoneal. Compositions suitable for other routes of administration are also encompassed by the invention, as described herein.

BTB transport protein modulators of use in the invention include any suitable BTB transport modulators. In some embodiments, the BTB transport protein modulator is one or more polyphenols. In some embodiments, the BTB transport protein modulator is one or more flavonoids. In some embodiments, the BTB transport protein modulator is quercetin.

In some embodiments the invention provides methods of treatment. In certain embodiments, the invention provides a method of treating a condition by administering to an animal suffering from the condition an effective amount of a calcineurin inhibitor and an amount of a BTB transport protein modulator, e.g., activator, sufficient to reduce or eliminate a side effect of the calcineurin inhibitor. In some embodiments the BTB transport protein modulator is a BTB transport protein activator. In some embodiments, the calcineurin inhibitor is tacrolimus or a tacrolimus analog. In certain embodiments the invention provides methods of treatment of organ transplant, an autoimmune disease, and an inflammatory disease with a calcineurin inhibitor, without or with reduced side effects by co-administering a modulator of a BTB transport protein in combination with the calcineurin inhibitor, thereby reducing or eliminating side effect to the calcineurin inhibitor. In some embodiments, the invention provides methods for treatment of organ transplant. Example or organ transplant include but are not limited to kidney transplant, pancreas transplant, liver transplant, heart transplant, lung transplant, intestine transplant, pancreas after kidney transplant, and simultaneous pancreas-kidney transplant. In other embodiments, the invention provides methods for the treatment of an autoimmune disease. Examples of autoimmune diseases include, but are not limited to, Rheumatoid Arthritis, Lupus nephritis, actopic dermatitis, and psoriasis. In yet other embodiments, the invention provides methods for the treatment of inflammatory diseases. Examples of inflammatory diseases include, but are not limited to, asthma, vulvar lichen sclerosis, chronic allergic contact dermatitis, eczema, vitiligo and ulcerative colitis

In some embodiments the invention provides methods of decreasing a side effect of a calcineurin inhibitor in an animal, e.g. a human, that has received an amount of the calcineurin inhibitor sufficient to produce a side effect by administering to the animal, e.g., human, an amount of a BTB transport protein modulator sufficient to reduce or eliminate the side effect.

Hence, in some embodiments, the methods and compositions of the present invention can be used to modulate transport of a variety of calcineurin inhibitors. In some embodiments, the dosage of the calcineurin inhibitor will be modulated according to the effect of the transport protein modulator. For instance, less calcineurin inhibitor may be needed to reach optimal effect when co-administered with the transport protein modulator. In other embodiments co-administering the transport protein modulator with a calcineurin inhibitor will allow for chronically administering the drug without drug escalation and/or without dependence on the drug. In another embodiment co-administering the transport protein modulator will allow for the elimination of a calcineurin inhibitor from a physiological compartment, e.g. wash out drug from central nervous system to reduce CNS effects. In some embodiments, the physiological compartment is a central nervous system. In some embodiments, the physiological compartment is a renal system. In some embodiments, the physiological compartment is a pancreatic system. In some embodiments, the physiological compartment is a hepatic system. In some embodiments, the physiological compartment is a fetal compartment.

In some embodiments the invention provides methods of decreasing a CNS, reproductive, gastrointestinal, pancreatic, renal and or hepatic effect of a calcineurin inhibitor in an animal, e.g. a human, that has received an amount of the calcineurin inhibitor sufficient to produce a side effect by administering to the animal, e.g., human, an amount of a BTB transport protein modulator sufficient to reduce or eliminate the side effect. The term “side effect,” as used herein, encompasses any effect of a substance, e.g. a CNS, pancreatic, renal and or hepatic effect. The effect may be acute or chronic. The effect may be biochemical, cellular, at the tissue level, at the organ level, at the multi-organ level, or at the level of the entire organism. The effect may manifest in one or more objective or subjective manners, any of which may be used to measure the effect. For some substances that may be normally or abnormally produced the effect may be a pathological effect.

In some embodiments, the CNS effect of a substance can be tremors, headache, changes in motor function, changes in mental status, changes in sensory functions, seizures, insomnia, paresthesia, dizziness, coma or delirium, as well as other effects mention herein, or combinations thereof. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof.

In some embodiments, methods and compositions of the invention decrease the effect of a calcineurin inhibitor on tissue metabolic function of an animal, e.g. a human, that has received an amount of the calcineurin inhibitor sufficient to produce a decrease in tissue metabolic function; the decrease in the effect of the calcineurin inhibitor is accomplished, for example, by administering to the animal, e.g., human, an amount of a BTB transport protein modulator sufficient to reduce or eliminate the decrease in tissue metabolic function. Without being bound by theory, and as an example only of a possible mechanism, it is thought that the methods and compositions of the invention operate by reducing or eliminating the concentration of the calcineurin inhibitor in the tissue where the decrease in tissue metabolic function is observed as a result of the treatment with calcineurin inhibitor. “Tissue metabolic function” as used herein includes the biochemical reactions that allow the tissue to perform its normal function such as anabolism, catabolism, movement, accumulation of molecules and the like.

In another embodiment co-administering the transport protein modulator will allow for a change in concentration of a calcineurin inhibitor in a physiological compartment, e.g. increase of calcineurin inhibitor in the periphery.

If an effect is measured objectively or subjectively (e.g., drowsiness, tremor, and the like), any suitable method for evaluation of objective or subjective effect may be used. Examples include visual and numeric scales and the like for evaluation by an individual. A further example includes sleep latency for measurement of drowsiness, or standard tests for measurement of concentration, mentation, memory, and the like. These and other methods of objective and subjective evaluation of side effects by either an objective observer, the individual, or both, are well-known in the art.

The term “fetal effect,” as used herein, encompasses any effect encompasses any effect of a substance that is introduced into the maternal system on the fetus. The effect may be acute or chronic. The effect may be biochemical, cellular, at the tissue level, at the organ level, at the multi-organ level, or at the level of the entire organism.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any combination thereof.

The term “physiological compartment” as used herein includes physiological structures, such as organs or organ groups or the fetal compartment, or spaces whereby a physiological or chemical barrier exists to exclude compounds or agents from the internal or external portion of the physiological structure or space. Such physiological compartments include the central nervous system, blood and other bodily fluids, the fetal compartment and internal structures contained within organs, such as the ovaries and testes.

### Compositions

In one aspect the invention provides compositions that include an agent, e.g., that reduces or eliminates a side and/or fetal effect of one or more calcineurin inhibitors. In some embodiments, the calcineurin inhibitor is co-administered with the agent that reduces the side effect. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

In some embodiments, the invention provides compositions containing a combination of a calcineurin inhibitor and an agent, e.g., that reduces or eliminates side and/or fetal effect of the calcineurin inhibitor. In some embodiments, the invention provides compositions containing a combination of a calcineurin inhibitor and an agent that changes the concentration in a physiological compartment of the calcineurin inhibitor. In some embodiments the invention provides pharmaceutical compositions that further include a pharmaceutically acceptable excipient. In some embodiments, the pharmaceutical compositions are suitable for oral administration. In some embodiments, the pharmaceutical compositions are suitable for transdermal administration. In some embodiments, the pharmaceutical compositions are suitable for injection. Other forms of administration are also compatible with embodiments of the pharmaceutical compositions of the invention, as described herein.

In some embodiments, the BTB transport protein is an ABC transport protein. In some embodiments, the BTB transport protein modulator is a BTB transport protein activator. In some embodiments, the BTB transport protein modulator is a BTB transport protein inhibitor. In some embodiments, the BTB transport protein modulator is a modulator of P-gP.

In some embodiments, the BTB transport protein modulator comprises a polyphenol. In other embodiments, a polyphenol which acts to lower a side effect of a calcineurin inhibitor through a non-BTB transport protein-mediated mechanism, or that acts to lower a side effect of a calcineurin inhibitor through a BTB transport protein-mediated mechanism and a non-BTB transport protein-mediated mechanism, is used. In other embodiments, a polyphenol which acts to increase a therapeutic effect of a calcineurin inhibitor through a non-BTB transport protein-mediated mechanism, or that acts to increase a therapeutic effect of a calcineurin inhibitor through a BTB transport protein-mediated mechanism and a non-BTB transport protein-mediated mechanism, is used. In other embodiments, a polyphenol which acts to increase the concentration of a calcineurin inhibitor in a physiological compartment through a non-BTB transport protein-mediated mechanism, or that acts to increase the concentration of a calcineurin inhibitor in a physiological compartment through a BTB transport protein-mediated mechanism and a non-BTB transport protein-mediated mechanism, is used. In some embodiments utilizing a polyphenol, the polyphenol is a flavonoid. In some embodiments utilizing a polyphenol, the polyphenol is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments utilizing a polyphenol, the polyphenol is a flavonol. In certain embodiments, the flavonol is selected from the group consisting of quercetin, galangin, and kaempferol, or combinations thereof. In some embodiments, the flavonol is quercetin. In some embodiments, the flavonol is galangin. In some embodiments, the flavonol is kaempferol.

In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof.

In some embodiments, the CNS effect of the calcineurin inhibitor that is reduced is selected from the group consisting of headache, tremor, imsonia, paresthesia, dizziness, abnormal dreams, agitation, amnesia, anxiety, confusion, convulsion, crying, depression, elevated mood, emotional lability, encephalopathy, haemorrhagic stroke, hallucinations, hypertonia, incoordination, monoparesis, myoclonus, nerve compression, nervousness, neuralgia, neuropathy, paralysis flaccid, psychomotor skills impaired, psychosis, quadriparesis, somnolence, thinking abnormal, vertigo, writing impaired, abnormal vision, amblyopia, ear pain, otitis media, tinnitus, carpal tunnel syndrome, cerebral infarction, hemiparesis, leukoencephalopathy, mental disorder, mutism, quadriplegia, speech disorder, syncope, drowsiness, impaired concentration, sexual dysfunction, sleep disturbances, habituation, dependence, alteration of mood, respiratory depression, nausea, vomiting, memory impairment, neuronal dysfunction, neuronal death, impaired mentation, tolerance, addiction, hallucinations, lethargy, myoclonic jerking, endocrinopathies, and combinations thereof. In some embodiments, the CNS effect of the calcineurin inhibitor that is reduced is selected from the group consisting of headache, tremor, imsonia, paresthesia, dizziness. In some embodiments, the CNS effect of the calcineurin inhibitor that is reduced is tremor. In some embodiments, the CNS effect of the calcineurin inhibitor that is reduced is headache. In some embodiments, the CNS effect of the calcineurin inhibitor that is reduced is imsonia. In some embodiments, the CNS effect of the calcineurin inhibitor that is reduced is paresthesia. In some embodiments, the CNS effect of the calcineurin inhibitor that is reduced is dizziness.

In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof.

In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof.

In some embodiments, the side effect is a decrease in tissue metabolic function.

In some embodiments the calcineurin inhibitor is CsA. In some embodiments the calcineurin inhibitor is tacrolimus. In some embodiments, the calcineurin inhibitor is tacrolimus analog. In some embodiments, the tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to decrease a side effect of the calcineurin inhibitor by a measurable amount, compared to the side effect without the BTB transport protein modulator, when the composition is administered to an animal. In some embodiments, a side effect of the calcineurin inhibitor is decreased by an average of at least about 5, 10, 15, 20, 25, 30, 35, 40, 45, 50, 55, 60, 65, 70, 75, 80, 85, 90, 95, or more than 95%, compared to the side effect without the BTB transport protein modulator. In some embodiments, a side effect of the calcineurin inhibitor is decreased by an average of at least about 5%, compared to the side effect without the BTB transport protein modulator. In some embodiments, a side effect of the calcineurin inhibitor is decreased by an average of at least about 10%, compared to the side effect without the BTB transport protein modulator. In some embodiments, a side effect of the calcineurin inhibitor is decreased by an average of at least about 15%, compared to the side effect without the BTB transport protein modulator. In some embodiments, a side effect of the calcineurin inhibitor is decreased by an average of at least about 20%, compared to the side effect without the BTB transport protein modulator. In some embodiments, a side effect is substantially eliminated compared to the side effect without the BTB transport protein modulator. “Substantially eliminated” as used herein encompasses no measurable or no statistically significant side effect (one or more side effects) of the calcineurin inhibitor, when administered in combination with the BTB transport protein modulator.

Thus, in some embodiments, the invention provides compositions that contain a polyphenol, e.g., a flavonol, and a calcineurin inhibitor, where the calcineurin inhibitor is present in an amount sufficient to exert an therapeutic effect and the polyphenol, e.g., a flavonol is present in an amount sufficient to decrease side effect of the calcineurin inhibitor by a measurable amount, compared to the side effect without the polyphenol, e.g., a flavonol when the composition is administered to an animal. The measurable amount may be an average of at least about 5%, 10%, 15%, 20%, or more than 20% as described herein. The side effect may be any side effect as described herein. In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof. In some embodiments, the side effect of the calcineurin inhibitor that is reduced is a CNS side effect. In some embodiments, the CNS effect of the calcineurin inhibitor that is reduced is selected from the group consisting of headache, tremor, imsonia, paresthesia, dizziness. In some embodiments, the CNS effect is tremor. In some embodiments, the CNS effect is headache. In some embodiments, the CNS effect is insomnia. In some embodiments, the CNS effect is paresthesia. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof. In some embodiments, the side effect is a decrease in tissue metabolic function.

In some embodiments, the invention provides compositions that contain a flavonol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and a calcineurin inhibitor that is tacrolimus, where the tacrolimus is present in an amount sufficient to exert a therapeutic effect and the flavonol is present in an amount sufficient to decrease a side effect of tacrolimus by a measurable amount, compared to the side effect without the flavonol when the composition is administered to an animal. The measurable amount may be an average of at least about 5%, 10%, 15%, 20%, or more than 20% as described herein. The side effect may be any side effect as described herein. In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof. In some embodiments, the side effect of the calcineurin inhibitor that is reduced is a CNS side effect. In some embodiments, the CNS effect of the calcineurin inhibitor that is reduced is selected from the group consisting of headache, tremor, imsonia, paresthesia, dizziness. In some embodiments, the CNS effect is tremor. In some embodiments, the CNS effect is headache. In some embodiments, the CNS effect is insomnia. In some embodiments, the CNS effect is paresthesia. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof. In some embodiments, the side effect is a decrease in tissue metabolic function.

In some embodiments, the invention provides compositions that contain a flavonol that is quercetin, galangin, or kaempferol, or combination thereof, and a calcineurin inhibitor that is tacrolimus, where tacrolimus is present in an amount sufficient to exert a therapeutic effect and the flavonol is present in an amount sufficient to decrease a side effect of tacrolimus by a measurable amount, compared to the side effect without the flavonol when the composition is administered to an animal. The measurable amount may be an average of at least about 5%, 10%, 15%, 20%, or more than 20% as described herein. The side effect may be any side effect as described herein. In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof. In some embodiments, the side effect of the calcineurin inhibitor that is reduced is a CNS side effect. In some embodiments, the CNS effect of the calcineurin inhibitor that is reduced is selected from the group consisting of headache, tremor, imsonia, paresthesia, dizziness. In some embodiments, the CNS effect is tremor. In some embodiments, the CNS effect is headache. In some embodiments, the CNS effect is insomnia. In some embodiments, the CNS effect is paresthesia. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof. In some embodiments, the side effect is a decrease in tissue metabolic function.

In some embodiments, the invention provides compositions that contains quercetin and tacrolimus where the tacrolimus is present in an amount sufficient to exert a therapeutic effect and the quercetin is present in an amount sufficient to decrease a side effect of tacrolimus by a measurable amount, compared to the side effect without the quercetin when the composition is administered to an animal. The measurable amount may be an average of at least about 5%, 10%, 15%, 20%, or more than 20% as described herein. The side effect may be any side effect as described herein. In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof. In some embodiments, the side effect of the calcineurin inhibitor that is reduced is a CNS side effect. In some embodiments, the CNS effect of the calcineurin inhibitor that is reduced is selected from the group consisting of headache, tremor, imsonia, paresthesia, dizziness. In some embodiments, the CNS effect is tremor. In some embodiments, the CNS effect is headache. In some embodiments, the CNS effect is insomnia. In some embodiments, the CNS effect is paresthesia. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof. In some embodiments, the side effect is a decrease in tissue metabolic function.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to increase a therapeutic effect of the calcineurin inhibitor by a measurable amount, compared to the therapeutic effect without the BTB transport protein modulator, when the composition is administered to an animal. In some embodiments, a therapeutic effect of the calcineurin inhibitor is increased by an average of at least about 5, 10, 15, 20, 25, 30, 35, 40, 45, 50, 55, 60, 65, 70, 75, 80, 85, 90, 95, or more than 95%, compared to the therapeutic effect without the BTB transport protein modulator. In some embodiments, a therapeutic effect of the calcineurin inhibitor is increased by an average of at least about 5%, compared to the therapeutic effect without the BTB transport protein modulator. In some embodiments, a therapeutic effect of the calcineurin inhibitor is increased by an average of at least about 10%, compared to the therapeutic effect without the BTB transport protein modulator. In some embodiments, a therapeutic effect of the calcineurin inhibitor is increased by an average of at least about 15%, compared to the therapeutic effect without the BTB transport protein modulator. In some embodiments, a therapeutic effect of the calcineurin inhibitor is increased by an average of at least about 20%, compared to the therapeutic effect without the BTB transport protein modulator. In some embodiments, a therapeutic effect is substantially increased compared to the therapeutic effect without the BTB transport protein modulator. “Substantially increased” as used herein encompasses a measurable or a statistically significant increase therapeutic effect (one or more therapeutic effects) of the calcineurin inhibitor, when administered in combination with the BTB transport protein modulator.

Thus, in some embodiments, the invention provides compositions that contain a polyphenol, e.g., a flavonol, and a calcineurin inhibitor, where the calcineurin inhibitor is present in an amount sufficient to exert an therapeutic effect and the polyphenol, e.g., a flavonol is present in an amount sufficient to increase a therapeutic effect of the calcineurin inhibitor by a measurable amount, compared to the therapeutic effect without the polyphenol, e.g., a flavonol when the composition is administered to an animal. The measurable amount may be an average of at least about 5%, 10%, 15%, 20%, or more than 20% as described herein.

In some embodiments, the invention provides compositions that contain a flavonol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and a calcineurin inhibitor that is tacrolimus, where the tacrolimus is present in an amount sufficient to exert a therapeutic effect and the flavonol is present in an amount sufficient to increase a therapeutic effect of tacrolimus by a measurable amount, compared to the therapeutic effect without the flavonol when the composition is administered to an animal. The measurable amount may be an average of at least about 5%, 10%, 15%, 20%, or more than 20% as described herein.

In some embodiments, the invention provides compositions that contain a flavonol that is quercetin, galangin, or kaempferol, or combination thereof, and a calcineurin inhibitor that is tacrolimus, where tacrolimus is present in an amount sufficient to exert a therapeutic effect and the flavonol is present in an amount sufficient to increase a therapeutic effect of tacrolimus by a measurable amount, compared to the therapeutic effect without the flavonol when the composition is administered to an animal. The measurable amount may be an average of at least about 5%, 10%, 15%, 20%, or more than 20% as described herein.

In some embodiments, the invention provides compositions that contains quercetin and tacrolimus where the tacrolimus is present in an amount sufficient to exert a therapeutic effect and the quercetin is present in an amount sufficient to increase a therapeutic effect of tacrolimus by a measurable amount, compared to the therapeutic effect without the quercetin when the composition is administered to an animal. The measurable amount may be an average of at least about 5%, 10%, 15%, 20%, or more than 20% as described herein.

In some embodiments, the BTB transport protein modulator is present in an amount sufficient to decrease a side effect of the calcineurin inhibitor by a measurable amount and to increase a therapeutic effect of the calcineurin inhibitor by a measurable amount, compared to the side effect and therapeutic effect without the BTB transport protein modulator, when the composition is administered to an animal. In some embodiments, a therapeutic effect of the calcineurin inhibitor is increased by an average of at least about 5, 10, 15, 20, 25, 30, 35, 40, 45, 50, 55, 60, 65, 70, 75, 80, 85, 90, 95, or more than 95%, compared to the therapeutic effect without the BTB transport protein modulator. In some embodiments, a therapeutic effect of the calcineurin inhibitor is increased by an average of at least about 5%, compared to the therapeutic effect without the BTB transport protein modulator. In some embodiments, a therapeutic effect of the calcineurin inhibitor is increased by an average of at least about 10%, compared to the therapeutic effect without the BTB transport protein modulator. In some embodiments, a therapeutic effect of the calcineurin inhibitor is increased by an average of at least about 15%, compared to the therapeutic effect without the BTB transport protein modulator. In some embodiments, a therapeutic effect of the calcineurin inhibitor is increased by an average of at least about 20%, compared to the therapeutic effect without the BTB transport protein modulator. In some embodiments, a therapeutic effect of the calcineurin inhibitor is increased by an average of at least about 30%, compared to the therapeutic effect without the BTB transport protein modulator. In some embodiments, a therapeutic effect of the calcineurin inhibitor is increased by an average of at least about 40%, compared to the therapeutic effect without the BTB transport protein modulator. In some embodiments, a therapeutic effect of the calcineurin inhibitor is increased by an average of at least about 50%, compared to the therapeutic effect without the BTB transport protein modulator.

Thus, in some embodiments, the invention provides compositions containing a BTB transport protein modulator present in an amount sufficient to decrease a side effect of a calcineurin inhibitor by an average of at least about 5% and to increase a therapeutic effect of the calcineurin inhibitor by an average of at least about 5%, compared to the side effect and therapeutic effect without the BTB transport protein modulator, when the composition is administered to an animal in combination with the calcineurin inhibitor. In some embodiments, the invention provides compositions containing a BTB transport protein modulator present in an amount sufficient to decrease a side effect of a calcineurin inhibitor by an average of at least about 10% and to increase a therapeutic effect of the calcineurin inhibitor by an average of at least about 10%, compared to the side effect and therapeutic effect without the BTB transport protein modulator, when the composition is administered to an animal in combination with the calcineurin inhibitor. In some embodiments, the invention provides compositions containing a BTB transport protein modulator present in an amount sufficient to decrease a side effect of a calcineurin inhibitor by an average of at least about 20% and to increase a therapeutic effect of the calcineurin inhibitor by an average of at least about 20%, compared to the side effect and therapeutic effect without the BTB transport protein modulator, when the composition is administered to an animal in combination with the calcineurin inhibitor. In some embodiments, the invention provides compositions containing a BTB transport protein modulator present in an amount sufficient to decrease a side effect of a calcineurin inhibitor by an average of at least about 10% and to increase a therapeutic effect of the calcineurin inhibitor by an average of at least about 20%, compared to the side effect and therapeutic effect without the BTB transport protein modulator, when the composition is administered to an animal in combination with the calcineurin inhibitor. In some embodiments, the invention provides compositions containing a BTB transport protein modulator present in an amount sufficient to decrease a side effect of a calcineurin inhibitor by an average of at least about 10% and to increase a therapeutic effect of the calcineurin inhibitor by an average of at least about 30%, compared to the side effect and therapeutic effect without the BTB transport protein modulator, when the composition is administered to an animal in combination with the calcineurin inhibitor. In some embodiments, the invention provides compositions containing a BTB transport protein modulator present in an amount sufficient to decrease a side effect of a calcineurin inhibitor by an average of at least about 10% and to increase a therapeutic effect of the calcineurin inhibitor by an average of at least about 40%, compared to the side effect and therapeutic effect without the BTB transport protein modulator, when the composition is administered to an animal in combination with the calcineurin inhibitor. In some embodiments, the invention provides compositions containing a BTB transport protein modulator present in an amount sufficient to decrease a side effect of a calcineurin inhibitor by an average of at least about 10% and to increase a therapeutic effect of the calcineurin inhibitor by an average of at least about 50%, compared to the side effect and therapeutic effect without the BTB transport protein modulator, when the composition is administered to an animal in combination with the calcineurin inhibitor.

In some embodiments, the invention provides compositions containing a polyphenol, e.g., a flavonol such as quercetin, present in an amount sufficient to decrease a side effect of a calcineurin inhibitor by an average of at least about 5% and to increase a therapeutic effect of the calcineurin inhibitor by an average of at least about 5%, when the composition is administered to an animal in combination with the calcineurin inhibitor, compared to the side effect and therapeutic effect without the polyphenol, e.g., flavonol such as quercetin. In some embodiments, the invention provides compositions containing a polyphenol, e.g., a flavonol such as quercetin present in an amount sufficient to decrease a side effect of a calcineurin inhibitor by an average of at least about 10% and to increase a therapeutic effect of the calcineurin inhibitor by an average of at least about 10%, when the composition is administered to an animal in combination with the calcineurin inhibitor, compared to the side effect and therapeutic effect when the calcineurin inhibitor is administered without the a polyphenol, e.g., a flavonol such as quercetin. In some embodiments, the invention provides compositions containing a polyphenol, e.g., a flavonol such as quercetin present in an amount sufficient to decrease a side effect of a calcineurin inhibitor by an average of at least about 20% and to increase a therapeutic effect of the calcineurin inhibitor by an average of at least about 20%, when the composition is administered to an animal in combination with the calcineurin inhibitor, compared to the side effect and therapeutic effect when the calcineurin inhibitor is administered without the a polyphenol, e.g., a flavonol such as quercetin. In some embodiments, the invention provides compositions containing a polyphenol, e.g., a flavonol such as quercetin present in an amount sufficient to decrease a side effect of a calcineurin inhibitor by an average of at least about 10% and to increase a therapeutic effect of the calcineurin inhibitor by an average of at least about 20%, when the composition is administered to an animal in combination with the calcineurin inhibitor, compared to the side effect and therapeutic effect when the calcineurin inhibitor is administered without the a polyphenol, e.g., a flavonol such as quercetin. In some embodiments, the invention provides compositions containing a polyphenol, e.g., a flavonol such as quercetin present in an amount sufficient to decrease a side effect of a calcineurin inhibitor by an average of at least about 10% and to increase a therapeutic effect of the calcineurin inhibitor by an average of at least about 30%, when the composition is administered to an animal in combination with the calcineurin inhibitor, compared to the side effect and therapeutic effect when the calcineurin inhibitor is administered without the polyphenol, e.g., a flavonol such as quercetin. In some embodiments, the invention provides compositions containing a polyphenol, e.g., a flavonol such as quercetin present in an amount sufficient to decrease a side effect of a calcineurin inhibitor by an average of at least about 10% and to increase a therapeutic effect of the calcineurin inhibitor by an average of at least about 40%, when the composition is administered to an animal in combination with the calcineurin inhibitor, compared to the side effect and therapeutic effect when the calcineurin inhibitor is administered without the polyphenol, e.g., a flavonol such as quercetin. In some embodiments, the invention provides compositions containing a polyphenol, e.g., a flavonol such as quercetin present in an amount sufficient to decrease a side effect of a calcineurin inhibitor by an average of at least about 10% and to increase a therapeutic effect of the calcineurin inhibitor by an average of at least about 50%, when the composition is administered to an animal in combination with the calcineurin inhibitor, compared to the side effect and therapeutic effect when the calcineurin inhibitor is administered without the a polyphenol, e.g., a flavonol such as quercetin.

In exemplary embodiments, the invention provides a composition that contains a polyphenol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or combinations thereof, and a calcineurin inhibitor, such as tacrolimus or a tacrolimus analog, where the calcineurin inhibitor is present in an amount sufficient to exert a therapeutic effect, and the polyphenol is present in an amount effective to decrease a side effect of the calcineurin inhibitor by a measurable amount (e.g., an average of at least about 5, 10, 15, 20, or more than 20%, as described herein) and to increase the therapeutic effect of the calcineurin inhibitor by a measurable amount (e.g., an average of at least about 5, 10, 15, 20, or more than 20%, as described herein). The side effect may be any side effect as described herein. In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof. In some embodiments, the side effect of the calcineurin inhibitor that is reduced is a CNS side effect. In some embodiments, the CNS effect of the calcineurin inhibitor that is reduced is selected from the group consisting of headache, tremor, imsonia, paresthesia, dizziness. In some embodiments, the CNS effect is tremor. In some embodiments, the CNS effect is headache. In some embodiments, the CNS effect is insomnia. In some embodiments, the CNS effect is paresthesia. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic nectosis, hepatotoxicity, liver fatty, venooclusive liver disease, diarrhea, nausea, constipation, vomiting, dyspepsia, anorexia, or LFT abnormal, as well as other effects mention herein, or combinations thereof. In some embodiments, the side effect is a decrease in tissue metabolic function.

In another exemplary embodiments, the invention provides a composition that contains quercetin and tacrolimus, where tacrolimus is present in an amount sufficient to exert a therapeutic effect, and the quercetin is present in an amount effective to decrease a side effect of tacrolimus by a measurable amount (e.g., an average of at least about 5, 10, 15, 20, or more than 20%, as described herein) and to increase the therapeutic effect of tacrolimus by a measurable amount (e.g., an average of at least about 5, 10, 15, 20, or more than 20%, as described herein). The side effect may be any side effect as described herein. In some embodiments, the side effect of the calcineurin inhibitor that is reduced is selected from the group consisting of CNS side effects, renal and/or urogenital side effects, reproductive system side effects, pancreatic, hepatic and/or gastrointestinal side effects, and combinations thereof. In some embodiments, the side effect of the calcineurin inhibitor that is reduced is a CNS side effect. In some embodiments, the CNS effect of the calcineurin inhibitor that is reduced is selected from the group consisting of headache, tremor, imsonia, paresthesia, dizziness. In some embodiments, the CNS effect is tremor. In some embodiments, the CNS effect is headache. In some embodiments, the CNS effect is insomnia. In some embodiments, the CNS effect is paresthesia. In some embodiments, the renal and/or urogenital side effect is selected from the group consisting of nephrotoxicity, renal function impairment, creatinine increase, urinary tract infection, oliguria, cystitis haemorrhagic, hemolytic-uremic syndrome or micturition disorder, as well as other effects mention herein, or combinations thereof. In some embodiments, the hepatic, pancreatic and/or gastrointestinal side effect is selected from the group consisting of hepatic necrosis, hepatotoxicity, liver fatty, venooclusive liver disease, diarrhea, nausea, constipation, vomiting, dyspepsia, anorexia, or LFT abnormal, as well as other effects mention herein, or combinations thereof. In some embodiments, the side effect is a decrease in tissue metabolic function.

In some embodiments, the invention provides a composition containing an calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to change the concentration in a physiological compartment of the calcineurin inhibitor by a measurable amount, compared to the concentration of the calcineurin inhibitor in the physiological compartment without the BTB transport protein modulator, when the composition is administered to an animal. In some embodiments, the BTB transport protein modulator decreases the concentration of a calcineurin inhibitor in a physiological compartment where a side effect is produced. In some embodiments, the concentration of the calcineurin inhibitor is decreased by an average of at least about 5, 10, 15, 20, 25, 30, 35, 40, 45, 50, 55, 60, 65, 70, 75, 80, 85, 90, 95, or more than 95%, compared to the concentration without the BTB transport protein modulator. In some embodiments, the concentration of the calcineurin inhibitor is decreased by an average of at least about 5%, compared to the concentration without the BTB transport protein modulator. In some embodiments, the concentration of the calcineurin inhibitor in a physiological compartment is decreased by an average of at least about 10%, compared to the concentration without the BTB transport protein modulator. In some embodiments, the concentration of the calcineurin inhibitor in a physiological compartment is decreased by an average of at least about 15%, compared to the concentration without the BTB transport protein modulator. In some embodiments, the concentration of the calcineurin inhibitor in a physiological compartment is decreased by an average of at least about 20%, compared to the concentration without the BTB transport protein modulator. In some embodiments, the concentration of a calcineurin inhibitor in a physiological compartment is substantially eliminated compared to the concentration without the BTB transport protein modulator. “Substantially eliminated” as used herein encompasses no measurable or no statistically significant concentration of the calcineurin inhibitor in a physiological compartment, when administered in combination with the BTB transport protein modulator.

Thus, in some embodiments, the invention provides compositions that contain a polyphenol, e.g., a flavonol, and an calcineurin inhibitor, where the calcineurin inhibitor is present in an amount sufficient to exert an therapeutic effect and the polyphenol, e.g., a flavonol is present in an amount sufficient to decrease the concentration of the calcineurin inhibitor in a physiological compartment by a measurable amount, compared to the concentration without the polyphenol, e.g., a flavonol when the composition is administered to an animal. The measurable amount may be an average of at least about 5%, 10%, 15%, 20%, or more than 20% as described herein. In some embodiments, the physiological compartment is a central nervous system. In some embodiments, the physiological compartment is a renal system. In some embodiments, the physiological compartment is a pancreatic system. In some embodiments, the physiological compartment is a hepatic system. In some embodiments, the physiological compartment is a fetal compartment.

In some embodiments, the invention provides compositions that contain a flavonol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and an calcineurin inhibitor that is tacrolimus, where tacrolimus is present in an amount sufficient to exert a therapeutic effect and the flavonol is present in an amount sufficient to decrease the concentration of tacrolimus in a physiological compartment by a measurable amount, compared to the concentration without the flavonol when the composition is administered to an animal. The measurable amount may be an average of at least about 5%, 10%, 15%, 20%, or more than 20% as described herein. In some embodiments, the physiological compartment is a central nervous system. In some embodiments, the physiological compartment is a renal system. In some embodiments, the physiological compartment is a pancreatic system. In some embodiments, the physiological compartment is a hepatic system. In some embodiments, the physiological compartment is a fetal compartment.

In some embodiments, the invention provides compositions that contain a flavonol that is quercetin, galangin, or kaempferol, or combination thereof, and an calcineurin inhibitor that is tacrolimus, where tacrolimus is present in an amount sufficient to exert a therapeutic effect and the flavonol is present in an amount sufficient to decrease the concentration of tacrolimus by a measurable amount, compared to the concentration without the flavonol when the composition is administered to an animal. The measurable amount may be an average of at least about 5%, 10%, 15%, 20%, or more than 20% as described herein. In some embodiments, the physiological compartment is a central nervous system. In some embodiments, the physiological compartment is a renal system. In some embodiments, the physiological compartment is a pancreatic system. In some embodiments, the physiological compartment is a hepatic system. In some embodiments, the physiological compartment is a fetal compartment.

In some embodiments, the invention provides compositions that contain quercetin and tacrolimus where tacrolimus is present in an amount sufficient to exert a therapeutic effect and the quercetin is present in an amount sufficient to decrease the concentration of tacrolimus in a physiological compartment by a measurable amount, compared to the concentration without quercetin when the composition is administered to an animal. The measurable amount may be an average of at least about 5%, 10%, 15%, 20%, or more than 20% as described herein. In some embodiments, the physiological compartment is a central nervous system. In some embodiments, the physiological compartment is a renal system. In some embodiments, the physiological compartment is a pancreatic system. In some embodiments, the physiological compartment is a hepatic system. In some embodiments, the physiological compartment is a fetal compartment.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to increase the concentration of the calcineurin inhibitor in a physiological compartment by a measurable amount, compared to the concentration of the calcineurin inhibitor without the BTB transport protein modulator, when the composition is administered to an animal. Examples of physiological compartments include, but are not limited to, blood, liver, lymph nodes, spleen, peyer's patches, intestines, lungs, heart, and kidney. In some embodiments, a concentration of the calcineurin inhibitor is increased by an average of at least about 5, 10, 15, 20, 25, 30, 35, 40, 45, 50, 55, 60, 65, 70, 75, 80, 85, 90, 95, or more than 95%, compared to the therapeutic effect without the BTB transport protein modulator. In some embodiments, concentration of the calcineurin inhibitor is increased by an average of at least about 5%, compared to the concentration of the calcineurin inhibitor without the BTB transport protein modulator. In some embodiments, concentration of the calcineurin inhibitor is increased by an average of at least about 10%, compared to the concentration of the calcineurin inhibitor without the BTB transport protein modulator. In some embodiments, concentration of the calcineurin inhibitor is increased by an average of at least about 15%, compared to the concentration of the calcineurin inhibitor without the BTB transport protein modulator. In some embodiments, a concentration of the calcineurin inhibitor is increased by an average of at least about 20%, compared to the concentration of the calcineurin inhibitor without the BTB transport protein modulator. In some embodiments, concentration of the calcineurin inhibitor is substantially increased compared to the concentration of the calcineurin inhibitor without the BTB transport protein modulator.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to increase the concentration of the calcineurin inhibitor in blood by a measurable amount, compared to the concentration of the calcineurin inhibitor without the BTB transport protein modulator, when the composition is administered to an animal.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to increase the concentration of the calcineurin inhibitor in a lymphoid tissue by a measurable amount, compared to the concentration of the calcineurin inhibitor without the BTB transport protein modulator, when the composition is administered to an animal. Examples of a lymphoid tissue include but are not limited to, thymus, bone marrow, lymph nodes, spleen, peyer's patches, and lymphatics.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to decrease the concentration of the calcineurin inhibitor in a organ, such as kidney, liver, lung or heart, by a measurable amount, compared to the concentration of the calcineurin inhibitor without the BTB transport protein modulator, when the composition is administered to an animal.

In some embodiments, the invention provides a composition containing a calcineurin inhibitor and a Blood-Tissue barrier (BTB) transport protein modulator, where the calcineurin inhibitor is present in an amount sufficient to exert a therapeutic effect and the BTB transport protein modulator is present in an amount sufficient to decrease the clearance of the calcineurin inhibitor from a physiological compartment where the calcineurin inhibitor exerts a therapeutic effect.

An “average” as used herein is preferably calculated in a set of normal human subjects, this set being at least about 3 human subjects, preferably at least about 5 human subjects, preferably at least about 10 human subjects, even more preferably at least about 25 human subjects, and most preferably at least about 50 human subjects.

In some embodiments, the invention provides a composition that contains a calcineurin inhibitor and a BTB transport protein modulator, e.g. a polyphenol such as a flavonoid. In some embodiments, the concentration of one or more of the calcineurin inhibitors and/or BTB transport protein modulator, e.g. a polyphenol such as a flavonol is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the calcineurin inhibitors and/or BTB transport protein modulator, e.g. a polyphenol such as a flavonoid is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the calcineurin inhibitors and/or BTB transport protein modulator, e.g. a polyphenol such as a flavonoid is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of one or more of the calcineurin inhibitors and/or BTB transport protein modulator, e.g. a polyphenol such as a flavonoid is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more of the calcineurin inhibitors and/or BTB transport protein modulator, e.g. a polyphenol such as a flavonoid is equal to or less than 10 g, 9.5 g, 9.0 g, 8.5 g, 8.0 g, 7.5 g, 7.0 g) 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more of the calcineurin inhibitors and/or BTB transport protein modulator, e.g. a polyphenol such as a flavonoid is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more of the calcineurin inhibitors and/or BTB transport protein modulator, e.g. a polyphenol such as a flavonoid is in the range of 0.0001-10 g, 0.0005-9 g, 0.001-8 g, 0.005-7 g, 0.01-6 g, 0.05-5 g, 0.1-4 g, 0.5-4 g, or 1-3 g.

In exemplary embodiments, compositions of the invention include quercetin and tacrolimus, where quercetin is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 200-700 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and tacrolimus is present in an amount from 0.01 to 200 mg, or about 0.1-160 mg, or about 0.1, 0.5, 1, 5, 10, 20, 50, 80, or 160 mg.

In some embodiments, tacrolimus/quercetin is present at about 0.1/50 mg (tacrolimus/quercetin). In some embodiments, tacrolimus is present at about 0.1 mg and the quercetin is present at about 100 mg. In some embodiments, tacrolimus is present at about 0.1 mg and the quercetin is present at about 200 mg. In some embodiments, tacrolimus is present at about 0.1 mg and the quercetin is present at about 300 mg. In some embodiments, tacrolimus is present at about 0.1 mg and the quercetin is present at about 1000 mg. In some embodiments, tacrolimus is present at about 0.5 mg and the quercetin is present at about 100 mg. In some embodiments, tacrolimus is present at about 0.5 mg and the quercetin is present at about 250 mg. In some embodiments, tacrolimus is present at about 0.5 mg and the quercetin is present at about 500 mg. In some embodiments, tacrolimus is present at about 0.5 mg and the quercetin is present at about 1000 mg. In some embodiments, tacrolimus is present at about 1 mg and the quercetin is present at about 100 mg. In some embodiments, tacrolimus is present at about 1 mg and the quercetin is present at about 250 mg. In some embodiments, tacrolimus is present at about 1 mg and the quercetin is present at about 500 mg. In some embodiments, tacrolimus is present at about 1 mg and the quercetin is present at about 1000 mg. In some embodiments, tacrolimus is present at about 5 mg and the quercetin is present at about 100 mg. In some embodiments, tacrolimus is present at about 5 mg and the quercetin is present at about 200 mg. In some embodiments, tacrolimus is present at about 5 mg and the quercetin is present at about 300 mg. In some embodiments, tacrolimus is present at about 5 mg and the quercetin is present at about 1000 mg. In some embodiments, tacrolimus is present at about 10 mg and the quercetin is present at about 100 mg. In some embodiments, tacrolimus is present at about 10 mg and the quercetin is present at about 200 mg. In some embodiments, tacrolimus is present at about 10 mg and the quercetin is present at about 300 mg. In some embodiments, tacrolimus is present at about 10 mg and the quercetin is present at about 1000 mg. In some embodiments, tacrolimus is present at about 15 mg and the quercetin is present at about 100 mg. In some embodiments, tacrolimus is present at about 15 mg and the quercetin is present at about 200 mg. In some embodiments, tacrolimus is present at about 15 mg and the quercetin is present at about 300 mg. In some embodiments, tacrolimus is present at about 15 mg and the quercetin is present at about 1000 mg.

In liquid preparations, tacrolimus can be present at about 1-100 mg/ml, or 1-50 mg/ml, or 1-20 mg/ml, or about 1, 5, 10, or 20 mg/ml and quercetin at about 1-1000 mg/ml, or about 10-1000 mg/ml, or about 50-1000 mg/ml, or about 100-1000 mg/ml, or about 1-500 mg/ml, or about 5-500 mg/ml, or about 50-500 mg/ml, or about 100-500 mg/ml, or about 200-1000 mg/ml, or about 200-800 mg/ml, or about 200-700 mg/ml, or about 10 mg/ml, or about 25 mg/ml, or about 50 mg/ml, or about 100 mg/ml, or about 200 mg/ml, or about 250 mg/ml, or about 300 mg/ml, or about 400 mg/ml, or about 500 mg/ml, or about 600 mg/ml, or about 700 mg/ml, or about 800 mg/ml, or about 900 mg/ml, or about 1000 mg/ml At higher levels of quercetin, solubility can be enhanced by adjusting the type of diluent.

In some embodiments, a molar ratio of one or more of the calcineurin inhibitors to the BTB transport protein modulator, e.g. a polyphenol such as a flavonoid can be 0.0001:1 to 1:1. Without limiting the scope of the invention, the molar ratio of one or more of the calcineurin inhibitors to the BTB transport protein modulator, e.g. a polyphenol such as a flavonoid can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

Without limiting the scope of the present invention, the molar ratio of one or more of the calcineurin inhibitors to the flavonoid can be about 0.03×10⁻⁵:1, 0.1×10⁻⁵:1, 0.04×10⁻³:1, 0.03×10⁻⁵:1, 0.02×10⁻⁵:1, 0.01×10⁻³:1, 0.1×10⁻³:1, 0.15×10⁻³:1, 0.2×10⁻³:1, 0.3×10⁻³:1, 0.4×10⁻³:1, 0.5×10⁻³: 1, 0.15×10⁻²: 1, 0.1×10⁻²:1, 0.2×10⁻²:1, 0.3×10⁻²:1, 0.4×10⁻²:1, 0.5×10⁻²:1, 0.6×10⁻²:1, 0.8×10⁻²:1, 0.01:1, 0.1:1; or 0.2:1 per dose. In one embodiment, the calcineurin inhibitor is tacrolimus. In one embodiment, the flavonoid is quercetin.

Without limiting the scope of the present invention, the molar ratio of one or more of the calcineurin inhibitors to the BTB transport protein modulator, e.g. a polyphenol such as a flavonoid can be about 0.001: 1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose. In one embodiment, the calcineurin inhibitor is tacrolimus. In one embodiment, the flavonoid is quercetin.

A. Pharmaceutical Compositions

The transport protein modulators of the invention are usually administered in the form of pharmaceutical compositions. The drugs described above are also administered in the form of pharmaceutical compositions. When the transport protein modulators and the drugs are used in combination, both components may be mixed into a preparation or both components may be formulated into separate preparations to use them in combination separately or at the same time.

This invention therefore provides pharmaceutical compositions that contain, as the active ingredient, a BTB transport protein modulator or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, as the active ingredient, a BTB transport protein modulator or a pharmaceutically acceptable salt and/or coordination complex thereof, a calcineurin inhibitor or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The BTB transport protein modulator and/or the calcineurin inhibitor may be prepared into pharmaceutical compositions in dosages as described herein (see, e.g., Compositions). Such compositions are prepared in a manner well known in the pharmaceutical art.

Pharmaceutical compositions for oral administration In some embodiments, the invention provides a pharmaceutical composition for oral administration containing a combination of a calcineurin inhibitor and an agent that reduces or eliminates a side and/or fetal effect of the calcineurin inhibitor, and a pharmaceutical excipient suitable for oral administration. In some embodiments, the agent that reduces or eliminates the side and/or fetal effect of the calcineurin inhibitor is a BTB transport protein modulator, e.g. a polyphenol such as a flavonol, as described elsewhere herein.

In some embodiments, the invention provides a solid pharmaceutical composition for oral administration containing:
(i) an effective amount of a calcineurin inhibitor;
(ii) an effective amount of an agent capable of reducing or eliminating one or more side effects of the calcineurin inhibitor; and
(iii) a pharmaceutical excipient suitable for oral administration.

In some embodiments, the composition further contains: (iv) an effective amount of a second calcineurin inhibitor.

In some embodiments, the pharmaceutical composition may be a liquid pharmaceutical composition suitable for oral consumption.

In some embodiments, the calcineurin inhibitor is tacrolimus. In some embodiments, the calcineurin inhibitor is a tacrolimus analog. In some embodiments, the calcineurin inhibitor is CsA. In some embodiments, the agent capable of reducing or eliminating one or more side effects of the calcineurin inhibitor is a BTB transport protein modulator, e.g., a BTB transport protein activator. In some embodiments, the agent capable of reducing or eliminating one or more side effects of the calcineurin inhibitor is a polyphenol, e.g., a flavonoid such as a flavonol.

In some embodiments, the invention provides a solid pharmaceutical composition for oral administration containing:
(i) an effective amount of a calcineurin inhibitor that is tacrolimus, tacrolimus analog or CsA;
(ii) an effective amount of a polyphenol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin; and
(iii) a pharmaceutical excipient suitable for oral administration.

In some embodiments, the composition further contains (iv) an effective amount of a second calcineurin inhibitor.

In some embodiments, the pharmaceutical composition may be a liquid pharmaceutical composition suitable for oral consumption.

In some embodiments, the invention provides a solid pharmaceutical composition for oral administration containing:
(i) an effective amount of a calcineurin inhibitor that is tacrolimus, tacrolimus analog or CsA;
(ii) an effective amount of a polyphenol that is quercetin, galangin, or kaempferol; and
(iii) a pharmaceutical excipient suitable for oral administration.

In some embodiments, the composition further contains (iv) an effective amount of a second calcineurin inhibitor.

In some embodiments, the pharmaceutical composition may be a liquid pharmaceutical composition suitable for oral consumption.

In some embodiments, the invention provides a solid pharmaceutical composition for oral administration containing an effective amount of tacrolimus, an amount of quercetin that is effective in reducing or eliminating a side effect of tacrolimus, and a pharmaceutically acceptable excipient. In some embodiments, the invention provides a liquid pharmaceutical composition for oral administration containing an effective amount of tacrolimus, an amount of quercetin that is effective in reducing or eliminating a side effect of tacrolimus, and a pharmaceutically acceptable excipient.

In some embodiments, the invention provides a solid pharmaceutical composition for oral administration containing tacrolimus at about 0.01-160 mg, quercetin at about 10-1000 mg and a pharmaceutically acceptable excipient. In some embodiments, the invention provides a liquid pharmaceutical composition for oral administration containing tacrolimus at about 0.1-200 mg/ml, quercetin at about 10-1000 mg/ml and a pharmaceutically acceptable excipient.

Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the calcineurin inhibitor and/or BTB transport protein modulator (e.g., flavonol) and to minimize precipitation of the calcineurin inhibitor and/or BTB transport protein modulator (e.g., flavonol). This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, 6-valerolactone and isomers thereof, α-butyrolactone and isomers thereof, and other solubilizers known in the art, such as dimethyl acetamide, dimethyl isosorbide, N-methylpyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, algimc acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical compositions for injection. In some embodiments, the invention provides a pharmaceutical composition for injection containing a combination of a calcineurin inhibitor and an agent that, e.g., reduces or eliminates a side and/or fetal effect of the calcineurin inhibitor, and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the transport protein modulator and/or the calcineurin inhibitor in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical compositions for topical (e.g. transdermal) delivery. In some embodiments, the invention provides a pharmaceutical composition for transdermal delivery containing a combination of a calcineurin inhibitor and an agent that, e.g., reduces or eliminates a side and/or fetal effect of the calcineurin inhibitor, and a pharmaceutical excipient suitable for transdermal delivery. In some embodiments, the agent that e.g., reduces or eliminates the side and/or fetal effect of the calcineurin inhibitor is a BTB transport protein modulator, e.g. a polyphenol such as a flavonol, as described elsewhere herein. Components and amounts of agents in the compositions are as described herein.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another preferred formulation for use in the methods of the present invention employs transdermal delivery devices (“patches”). Such transdermal patches may be used to provide continuous or discontinuous infusion of the transport protein modulator in controlled amounts, either with or without calcineurin inhibitor. Thus, in some embodiments the invention provides a transdermal patch incorporating a BTB transport protein modulator, e.g., a polyphenol such as a flavonoid (e.g., quercetin). In some embodiments the invention provides a transdermal patch incorporating a BTB transport protein modulator, e.g., a polyphenol such as a flavonoid (e.g., quercetin) in combination with a calcineurin inhibitor, e.g. tacrolimus.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Other pharmaceutical compositions. Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

B. Kits

The invention also provides kits. The kits include an agent as described herein, in suitable packaging, and written material that can include instructions for use, discussion of clinical studies, listing of side effects, and the like. The kit may further contain a calcineurin inhibitor. In some embodiments, the calcineurin inhibitor and the agent are provided as separate compositions in separate containers within the kit. In some embodiments, the calcineurin inhibitor and the agent are provided as a single composition within a container in the kit. Suitable packaging and additional articles for use (e.g., measuring cup for liquid preparations, foil wrapping to minimize exposure to air, and the like) are known in the art and may be included in the kit.

### Methods

In another aspect, the invention provides methods, including methods of treatment, methods of decreasing or increasing the concentration of a substance in a physiological compartment, methods of enhancing a therapeutic effect of a substance, methods of drug wash-out, and methods for identifying modulators of Blood-Tissue barrier transport proteins.

For simplicity, methods will be described in terms of reduction of a side effect of a calcineurin inhibitor. It is understood that the methods apply equally to exclusion of a substance from the fetal compartment, or reduction of fetal effects of a calcineurin inhibitor.

The term “animal” or “animal subject” as used herein includes humans as well as other mammals. The methods generally involve the administration of one or more drugs for the treatment of one or more diseases. Combinations of agents can be used to treat one disease or multiple diseases or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

In some embodiments, the invention provides a method of treating a condition by administering to an animal suffering from the condition an effective amount of a calcineurin inhibitor and an amount of a BTB transport protein activator sufficient to reduce or eliminate a side effect of the calcineurin inhibitor. In some embodiments, the activator reduces or eliminates a plurality of side effects of the calcineurin inhibitor.

In some embodiments, the invention provides a method of treating a condition by administering to an animal suffering from the condition an effective amount of a calcineurin inhibitor and an amount of a BTB transport protein activator sufficient to increase a therapeutic effect of the calcineurin inhibitor. In some embodiments, the activator increases a plurality of therapeutic effects of the calcineurin inhibitor.

In some embodiments, the invention provides a method of treating a condition by administering to an animal suffering from the condition an effective amount of a calcineurin inhibitor and an amount of a BTB transport protein activator sufficient to decrease or increase the concentration of the calcineurin inhibitor in a physiological compartment.

In some embodiments the animal is a mammal, e.g., a human.

In some embodiments, the invention provides a method of treating a condition by administering to an animal suffering from the condition an effective amount of a calcineurin inhibitor and an amount of a BTB transport protein activator sufficient to increase a therapeutic effect of a calcineurin inhibitor in a physiological compartment.

The calcineurin inhibitor and the BTB transport protein activator are co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present. Thus, in some embodiments, the BTB transport protein activator and the calcineurin inhibitor are administered in a single composition. In some embodiments, the calcineurin inhibitor and the BTB transport protein activator are admixed in the composition. Typically, the calcineurin inhibitor is present in the composition in an amount sufficient to produce a therapeutic effect, and the BTB transport protein activator is present in the composition in an amount sufficient to reduce a side effect of the calcineurin inhibitor and/or decrease or increase the concentration of the calcineurin inhibitor in a physiological compartment and/or increase a therapeutic effect of the calcineurin inhibitor. In some embodiments, the calcineurin inhibitor is present in an amount sufficient to exert a therapeutic effect and the BTB transport protein activator is present in an amount sufficient to decrease a side effect of the calcineurin inhibitor by an average of at least about 5, 10, 15, 20, 25, 30, 40, 50, 60, 70, 80, 90, more than 90%, or substantially eliminate a side effect, compared to the effect without the BTB transport protein activator.

Administration of the calcineurin inhibitor and the agent, e.g., that reduces or eliminates at least one side effect of the calcineurin inhibitor may be by any suitable means. If the agents are administered as separate compositions, they may be administered by the same route or by different routes. If the agents are administered in a single composition, they may be administered by any suitable route. In some embodiments, the agents are administered as a single composition by oral administration. In some embodiments, the agents are administered as a single composition by transdermal administration. In some embodiments, the agents are administered as a single composition by injection.

In some embodiments, the agent that reduces or eliminates a side effect of a calcineurin inhibitor is a BTB transport protein modulator, BTB transport protein modulators are as described herein. In some embodiments, a polyphenol is used. In some embodiments, a flavonoid is used. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin, kaempferol, or galangin. In some embodiments, the flavonoid is quercetin. Dosages are as provided for compositions. Typically, the daily dosage of the BTB transport protein modulator will be about 0.5-100 mg/kg.

The calcineurin inhibitor may be any calcineurin inhibitor described herein. In some embodiments, the calcineurin inhibitor is tacrolimus or a tacrolimus analog, as described herein.

The methods of the invention may be used for treatment of any suitable condition, e.g., organ transplant, an autoimmune disease, and an inflammatory disease, where one or more calcineurin inhibitors are used that have CNS effects.

For example, in some embodiments, the methods of the invention include the treatment of organ transplant recipient to prevent organ rejection by administering to an animal in need of treatment an effective amount of a calcineurin inhibitor, such as tacrolimus, and an effective amount of an agent that reduces or eliminates a side effect of the calcineurin inhibitor. Example of organ transplant include, but are not limited to, kidney transplant, pancreas transplant, liver transplant, heart transplant, lung transplant, intestine transplant, pancreas after kidney transplant, and simultaneous pancreas-kidney transplant.

In other embodiments, the methods of the invention include the treatment of an autoimmune disease by administering to an animal in need of treatment an effective amount of a calcineurin inhibitor, such as tacrolimus, and an effective amount of an agent that reduces or eliminates a side effect of the calcineurin inhibitor. Examples of autoimmune diseases include, but are not limited to, Lupus nephritis, actopic dermatitis, and psoriasis.

In yet other embodiments, the methods of the invention include the treatment of inflammatory conditions rejection by administering to an animal in need of treatment an effective amount of a calcineurin inhibitor, such as tacrolimus, and an effective amount of an agent that reduces or eliminates a side effect of the calcineurin inhibitor. Examples of inflammatory conditions include, but are not limited to, asthma, vulvar lichen sclerosis, chronic allergic contact dermatitis, eczema, vitiligo and ulcerative colitis.

When a calcineurin inhibitor and an agent as described herein are used in combination, any suitable ratio of the two agents, e.g., molar ratio, wt/wt ration, wt/volume ratio, or volume/volume ratio, as described herein, may be used.

The invention further provides methods of reversing one or more side effects of a calcineurin inhibitor by administering a BTB transport protein activator to an animal that has received an amount of the calcineurin inhibitor sufficient to produce one or more side effects. The methods are especially useful in a situation where it is desired to rapidly reverse one or more side effects of a calcineurin inhibitor, e.g., in an overdose situation. Any suitable BTB transport protein described herein may be used.

In some embodiments, the invention provides a method for reversing a side effect of a calcineurin inhibitor in a human by administering to the human an amount of a BTB transport protein modulator sufficient to partially or completely reverse a side effect of the calcineurin inhibitor, where the human has received an amount of said calcineurin inhibitor sufficient to produce a side effect. In some embodiments, the human has received an overdose of the calcineurin inhibitor producing the side effect. In some embodiments, the individual continues to experience peripheral effects of the calcineurin inhibitor. In some embodiments, the BTB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin. Typically, the flavonoid will be administered by injection, e.g., intravenously or intraperitoneally, in a dose sufficient to partially or completely reverse a side effect of the calcineurin inhibitor. Such a dose in a human can be, e.g., about 0.1-100 g, or about 0.5-50 g, or about 1-20 g, or 1, 2, 3, 4, 5, 6, 7, 8, 9, 10, 12, 14, 16, 18, or 20 g. In general, the dose can be 0.01-1.5 g/kg.

The invention further provides methods of increasing one or more therapeutic effects of a calcineurin inhibitor by administering a BTB transport protein activator to an animal that has received an amount of the calcineurin inhibitor sufficient to produce one or more therapeutic effects.

In some embodiments, the invention provides a method for increasing a therapeutic effect of a calcineurin inhibitor in a human by administering to the human an amount of a BTB transport protein modulator sufficient to partially or completely reverse a side effect of the calcineurin inhibitor, where the human has received an amount of said calcineurin inhibitor sufficient to produce a therapeutic effect. In some embodiments, there is an increase in the therapeutic effect of the calcineurin inhibitor with increase in dose of the BTB transport protein modulator. In some embodiments, there is a window to the increase in the therapeutic effect of the calcineurin inhibitor in which the therapeutic effect increase with increase in dose of the BTB transport protein modulator to a certain point, but then there is a decrease in the therapeutic effect with further increases in dose of the BTB transport protein modulator. In some embodiments, the BTB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin. Typically, the flavonoid will be administered by injection, e.g., intravenously or intraperitoneally, in a dose sufficient to increase a therapeutic effect of the calcineurin inhibitor. Such a dose in a human can be, e.g., about 0.1-100 g, or about 0.5-50 g, or about 1-20 g, or 1, 2, 3, 4, 5, 6, 7, 8, 9, 10, 12, 14, 16, 18, or 20 g. In general, the dose can be 0.01-1.5 g/kg. In general the dose can be 0.02-0.5 g/kg. In general the dose can be 0.15-0.5 g/kg.

The invention further provides methods of methods of decreasing or increasing the concentration of a calcineurin inhibitor in a physiological compartment by administering a BTB transport protein activator to an animal that has received an amount of the calcineurin inhibitor sufficient decrease or increase the concentration of a calcineurin inhibitor in a physiological compartment.

In some embodiments, the invention provides a method for decreasing or increasing the concentration of a calcineurin inhibitor in a physiological compartment in a human by administering to the human an amount of a BTB transport protein modulator sufficient to decrease or increase the concentration of a calcineurin inhibitor in a physiological compartment, where the human has received an amount of said calcineurin inhibitor sufficient for treatment. In some embodiments, the invention provides a method for decreasing or increasing the concentration of tacrolimus or a tacrolimus analog in a physiological compartment in a human by administering to the human an amount of a BTB transport protein modulator sufficient to decrease or increase the concentration of tacrolimus or a tacrolimus analog in a physiological compartment, where the human has received an amount of tacrolimus or a tacrolimus analog sufficient for treatment. In some embodiments, the BTB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin. Typically, the flavonoid will be administered by injection, e.g., intravenously or intraperitoneally, in a dose sufficient to increase a therapeutic effect of the calcineurin inhibitor. Such a dose in a human can be, e.g., about 0.1-100 g, or about 0.5-50 g, or about 1-20 g, or 1, 2, 3, 4, 5, 6, 7, 8, 9, 10, 12, 14, 16, 18, or 20 g. In general, the dose can be 0.01-1.5 g/kg. In general the dose can be 0.02-0.5 g/kg. In general the dose can be 0.15-0.5 g/kg.

A further aspect of the invention is a method of identifying a transport protein modulator. A drug is administered in an appropriate animal model in the presence and absence of a test compound and the concentration of the drug in a biological sample are measured. The test compound is identified as a transport protein modulator if the concentration of the drug in the biological sample is lower in the presence of the test compound. In some embodiments, the biological sample may be intraventricular samples, amniotic fluid, chorionic samples or brain parenchymal samples. Moreover, the animal model may be a rodent, such as mice or rats, or a primate, horse, dog, sheep, goat, rabbit, or chicken. In other embodiments, the animal model possesses a mutant form of a blood brain transporter.

Administration

The methods involve the administration of an agent as described herein. For simplicity, administration will be described in terms of reduction of a side effect of a calcineurin inhibitor. It is understood that the administration apply equally to other methods described herein.

In some embodiments, a calcineurin inhibitor that produces a side effect is administered in combination with an agent that reduces the side effects of the calcineurin inhibitor. In some embodiments, other agents are also administered, e.g., other calcineurin inhibitors. When two or more agents are co-administered, they may be co-administered in any suitable manner, e.g., as separate compositions, in the same composition, by the same or by different routes of administration.

In some embodiments, the agent that reduces or eliminates a side effect of a calcineurin inhibitor is administered in a single dose. This may be the case, e.g., in wash-out methods where the agent is introduced into an animal to quickly lower the side effect of a calcineurin inhibitor already present in the body. Typically, such administration will be by injection, e.g., intravenous injection, in order to introduce the agent quickly. However, other routes may be used as appropriate. A single dose of an agent that reduces or eliminates a side effect of a calcineurin inhibitor may also be used when it is administered with the calcineurin inhibitor (e.g., a calcineurin inhibitor that produces a CNS effect) for treatment of an acute condition.

In some embodiments, the agent that reduces or eliminates a side effect of a substance and/or calcineurin inhibitor is administered in multiple doses. Dosing may be about once, twice, three times, four times, five times, six times, or more than six times per day. Dosing may be about once a month, once every two weeks, once a week, or once every other day. In one embodiment the calcineurin inhibitor is tacrolimus. In another embodiment the calcineurin inhibitor and the transport protein activator are administered together about once per day to about 6 times per day. In another embodiment the administration of the calcineurin inhibitor and the transport protein activator continues for less than about 7 days. In yet another embodiment the administration continues for more than about 6, 10, 14, 28 days, two months, six months, or one year. In some cases, continuous dosing is achieved and maintained as long as necessary, e.g., organ transplant patient.

Administration of the agents of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

An effective amount of a transport protein modulator and an effective amount of a calcineurin inhibitor may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The BTB transport protein modulator and the calcineurin inhibitor may be administered in dosages as described herein (see, e.g., Compositions). Dosing ranges for calcineurin inhibitors are known in the art. It is also known in the art that due to intersubject variability in calcineurin inhibitors, such as tacrolimus, pharmacokinetics, individualization of dosing regimen is necessary for optimal therapy. Dosing for the BTB transport modulator may be found by routine experimentation. For a flavonoid, e.g., quercetin, typical daily dose ranges are, e.g. about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 50-4000 mg, or about 50-3000 mg, or about 50-2000 mg, or about 50-1000 mg, or about 50-500 mg, or about 50-100 mg, about 100-5000 mg, or about 100-4000 mg, or about 100-3000 mg, or about 100-2000 mg, or about 100-1000 mg, or about 100-500 mg. In some embodiments, the daily dose of quercetin is about 100, 200, 300, 400, 500, 600, 700, 800, 900, or 1000 mg. In some embodiments, the daily dose of quercetin is 100 mg. In some embodiments, the daily dose of quercetin is 500 mg. In some embodiments, the daily dose of quercetin is 1000 mg. Daily doses may be administered in single or multiple doses. For instance, in some embodiments the BTB transport modulator is administered 3 times per day of an oral dose of 500 mg. In other embodiments the BTB transport modulator is administered 3 times per day of an i.v. dose of 150 mg. Daily doses of quercetin may be administered in the same or separate composition as the calcineurin inhibitor. In some embodiments, the BTB transport protein modulator is in the bloodstream 30 minutes prior to the therapeutic agent. This may be accomplished by administering the BTB transport modulator separately from the calcineurin inhibitor or by administering the BTB transport modulator and calcineurin inhibitor in the same composition that is formulated so that the BTB transport modulator reaches the bloodstream before the calcineurin inhibitor. Daily dose range may depend on the form of flavonoid, e.g., the carbohydrate moieties attached to the flavonoid, and/or factors with which the flavonoid is administered, as described herein. The serum half-life for, e.g., quercetin, is about 19-25 hours, so single dose accuracy is not crucial.

When a BTB transport modulator, e.g., a flavonoid such as quercetin, is administered in a composition that comprises one or more calcineurin inhibitors, and the calcineurin inhibitor has a shorter half-life than BTB transport modulator unit dose forms of the calcineurin inhibitor and the BTB transport modulator may be adjusted accordingly. Thus, for example, if quercetin is given in a composition also containing, e.g., calcineurin inhibitor, a typical unit dose form is, e.g., 50 mg calcineurin inhibitor/100 mg quercetin, or 50 mg calcineurin inhibitor/500 mg quercetin. See e.g., Compositions.

When a BTB transport protein that is the target of the BTB transport modulator is present on the cells where the calcineurin inhibitor is exerting its therapeutic effect, unit dose forms of the BTB transport modulator may be adjusted such that the side effect of the calcineurin inhibitor are reduced without a substantial reduction of the therapeutic effect.

### EXAMPLES

### Example 1

### Human Study of the Effects of Quercetin (Q) and Tacrolimus on Transplant Patients

An empiric trial on the effects of oral quercetin (Q) on tacrolimus CNS effects can be conducted. Inclusion criteria include patients who have received liver, kidney and heart transplantation, under tacrolimus treatment who demonstrate neurotoxic episodes such as seizures, tremors, headache, and abnormal vision. Preferably, these patients would have no history of prior transplantation or of the CNS effects associated with tacrolimus. The Table, below, provides exemplary dosing schemes for tacrolimus.

| | Tacrolimus |
|---|---|
| | Route |
| Population | (Dose) |
| Kidney Transplant | IV |
| | (0.02 mg/kg/12 hr |
| | Oral |
| | (0.3 mg/kg/day) |
| Liver Transplant | IV |
| | (0.05 mg/kg/12 hr) |
| | Oral |
| | (0.3 mg/kg/day |
| Heart Transplant | IV |
| | (0.01 mg/kg/day) |
| | Oral |
| | (0.15 mg/kg/day) |

Due to intersubject variability in tacrolimus pharmacokinetics, individualization of dosing regimen is necessary for optimal therapy. The dose of tacrolimus is adjusted daily to achieve a trough concentration of 15 to 20 and approximately 10 ng/mL in the first 2 weeks and the subsequent 2 weeks, respectively. A blood sample is collected before the morning dose for measuring the concentrations. The tacrolimus whole-blood concentration is measured using the microparticle enzyme immunoassay method, which is known in the art.

Q 100-500 mg per gel capsule is compounded and supplied to all subjects. In some trials, placebo capsules are also compounded. Subjects are instructed to complete daily diaries for 7 days and continue their baseline medications and regular activities. On approximately the 7th day, they are asked to begin twice daily dosing of 2 Q (200-1000 mg) capsules (total daily dose of Q, 400-2000 mg), or an equivalent dosage of placebo, preferably double-blinded (if placebo is used). Diaries are then completed for 7 days. Individual diaries include rating sleep interference, focus, and other general CNS symptoms, as well as CNS symptoms specific to tacrolimus, such as seizures, tremors, headache, and abnormal vision, over the prior 24 hours. Subjects are instructed that concomitant medications should not be altered without speaking with the investigator. Subjects are advised that they will be contacted every day or every other day to assess progress in the trial and any side effects associated with the addition of Q. At the end of the trial, patients are interviewed. They are asked to rate their satisfaction with the study medication (−2-+2) and its ability to modulate the CNS effects of tacrolimus. If the study has used placebo and is blinded, the blind is broken and statistical comparisons of Q versus placebo are performed.

### Example 2

### Human Study of the Effects of Quercetin (Q) and Tacrolimus on Atopic Dermatitis Patient

An empiric trial on the effects of oral quercetin (Q) on tacrolimus CNS effects can be conducted. Inclusion criteria include patients who suffered from actopic dermatitis and are under PROTOPIC Ointment (tacrolimus) and that demonstrate neurotoxic episodes such as seizures, tremors, abnormal vision etc. . . . Patients can apply PROTOPIC Ointment 0.03% or PROTOPIC Ointment 0.01% to the affected skin twice daily.

Q 100-500 mg per gel capsule is compounded and supplied to all subjects. In some trials, placebo capsules are also compounded. Subjects are instructed to complete daily diaries for 7 days and continue their baseline medications and regular activities. On approximately the 7th day, they are asked to begin twice daily dosing of 2 Q (200-1000 mg) capsules (total daily dose of Q, 200-2000 mg), or an equivalent dosage of placebo, preferably double-blinded (if placebo is used). Diaries are then completed for 7 days. Individual diaries include rating sleep interference, focus, and other general CNS symptoms, as well as CNS symptoms specific to tacrolimus, such as seizures, tremors, headache, and abnormal vision, over the prior 24 hours. Subjects are instructed that concomitant medications should not be altered without speaking with the investigator. Subjects are advised that they will be contacted every day or every other day to assess progress in the trial and any side effects associated with the addition of Q. At the end of the trial, patients are interviewed. They are asked to rate their satisfaction with the study medication (−2-+2) and its ability to modulate the CNS effects of tacrolimus. If the study has used placebo and is blinded, the blind is broken and statistical comparisons of Q versus placebo are performed.

### Example 3

### BTB Transport Protein Activator Increases Tacrolimus Efficacy

Animals: 8-9 weeks-old Lewis and Brown Norway male rats were obtained from Charles River Laboratories. General procedures for animal care and housing was in accordance with the National Research Council (NRC) Guide for the Care and Use of Laboratory Animals (1996) and the Animal Welfare Standards incorporated in 9 CFR Part 3, 1991.

Treatment: Lewis rats were treated with different single doses of LNS 0694i.p. 30 minutes prior to single i.v. injections of tacrolimus at a concentration of 1 mg/kg as described in the table below.

| | LNS 0694 ™ | |
|---|---|---|
| | (BTB Transport | |
| | Protein Activator) | FK506 |
| | Treatment | Treatment |
| Group | (IP) | (IV) |
| 1 | Baseline | — |
| | (Untreated Control) | |
| 2 | 50 mg/kg | 1 mg/kg |
| 3 | 150 mg/kg | 1 mg/kg |
| 4 | 300 mg/kg | 1 mg/kg |
| 5 | — | 1 mg/kg |

Dose calculations (mg/kg) were based on the individual body weight measured on the day of treatment.

Spleens were collected at 4 hr after administration of FK506 for use in the in vitro mixed lymphocyte reaction (MLR) and Con A assays. In addition to treated Lewis rats, spleens were collected from 5 Brown Norway rats (not treated) which were used for the in vitro assay.

Mixed lymphocyte reactions: A single cell suspension of the spleen of each rat on test (LEW, responder) was prepared with a Dounce homogenizer and wash-medium. The cell suspension was depleted of red blood cells (treatment with NH4CL/Tris buffer) and washed twice with wash medium before resuspending in complete medium (CM; RPMI 1640 with 5% heat-inactivated (30 minutes at 56° C.) normal rat serum (from Lewis rats), 2 mM GlutaMAX, 100 U/ml penicillin and 100 μg/ml streptomycin mixture, and 55 μM 2-mercaptoethanol). A single cell suspension of splenocytes from five Brown Norway rats (BN, stimulators) was prepared with the same method. The BN splenocytes were pooled and irradiated with 1,500-2,000 rad (cesium source) before use.

Varying numbers of responder cells were mixed with a constant number of stimulators (105) in 96-well, U-bottom cell culture plates to give a final responder:stimulator (R:S) ratio of 10:1,5: 1, 1:1 and 0.5:1 in 200 μl CM. Control wells for each cell suspension contained 10⁵ responders alone in medium and 10⁵ pooled, irradiated stimulators alone in medium (separate wells). For a positive proliferative response, each responder (10⁵) was treated with 2.5 μg/ml Con A.

Cultures were incubated at 37±°1 C. for 72±2 hours in 5±1% CO₂ humidified air. Each well was pulsed with 1 μCi tritiated thymidine for 18±2 hours before automatic harvesting and analysis in a liquid scintillation counter.

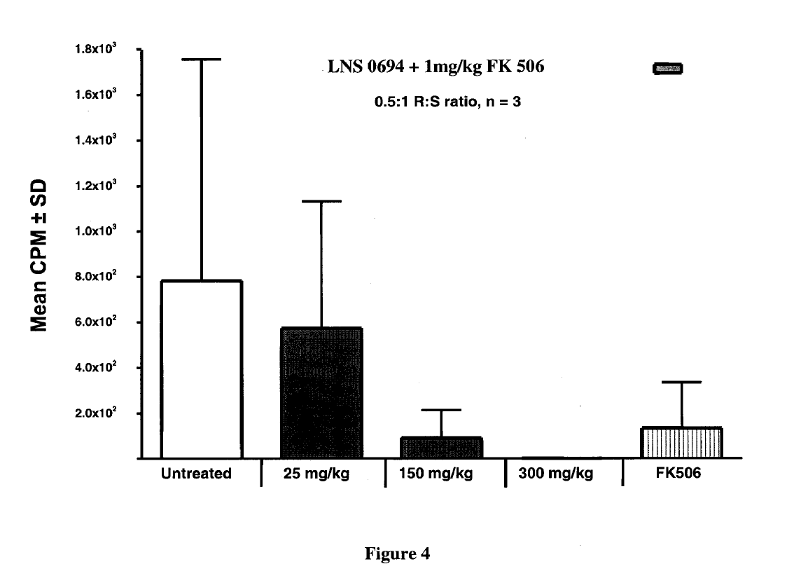

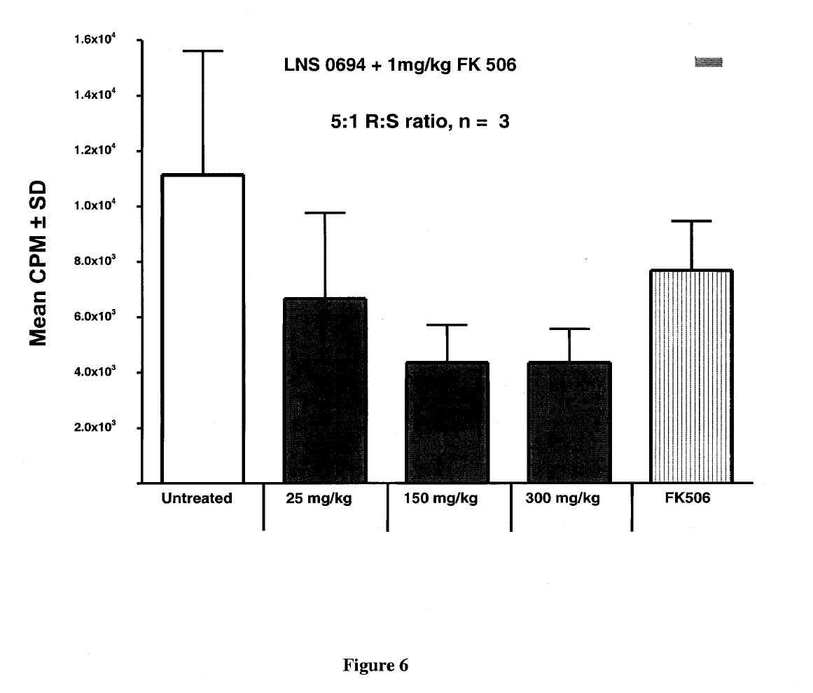

The results of the MLR assays [thymidine incorporation as counts per minute (CPM)] are expressed as mean ±SD. Results are shown in FIGS. 4-7.

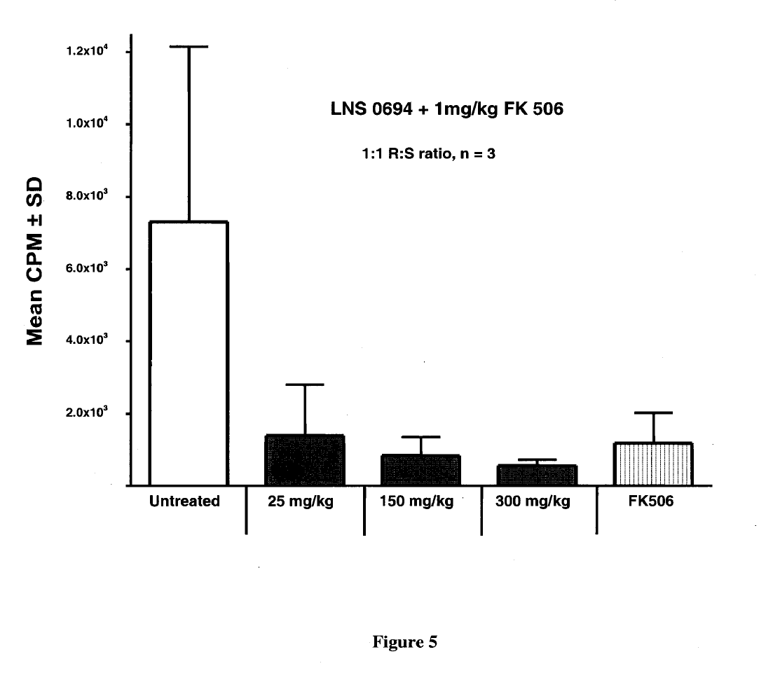

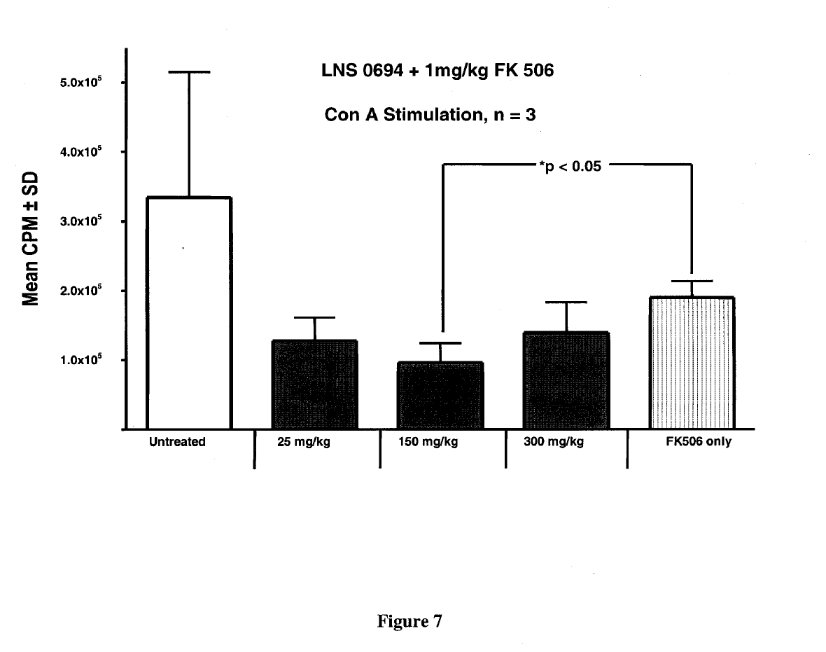

Results: The effect of BTB transport protein activator on FK 506-inhibitory effects on lymphocyte proliferation was evaluated by mixing LEW, responder and allogeneic BN, stimulators at three different ratios. As shown in FIGS. 4-6, untreated LEW, responders' proliferation increased with increases in R:S ratio (FIG. 4-6). As expected, FK 506 inhibited MLR. FK 506 exhibited a stronger inhibitory effect at lower R:S ratios (See FIGS. 4 and 5). LNS 0694 increased FK 506 inhibitory effect in a dose-dependent manner. As shown in FIG. 7, LNS 0694 also increased FK 506 inhibitory effect when the LEW, responders were activated with Con A.

These results suggest that FK 506 efficacy is enhanced when combine with a BTB transport protein modulator.

### Example 4

### BTB Transport Protein does not Impair Tacrolimus Induced T Cell Suppression In Vitro

Animals: 8-9 weeks-old Lewis and Brown Norway male rats were obtained from Charles River Laboratories. General procedures for animal care and housing was in accordance with the National Research Council (NRC) Guide for the Care and Use of Laboratory Animals (1996) and the Animal Welfare Standards incorporated in 9 CFR Part 3, 1991.

Spleens were collected for use in in vitro Con A and LPS assays.

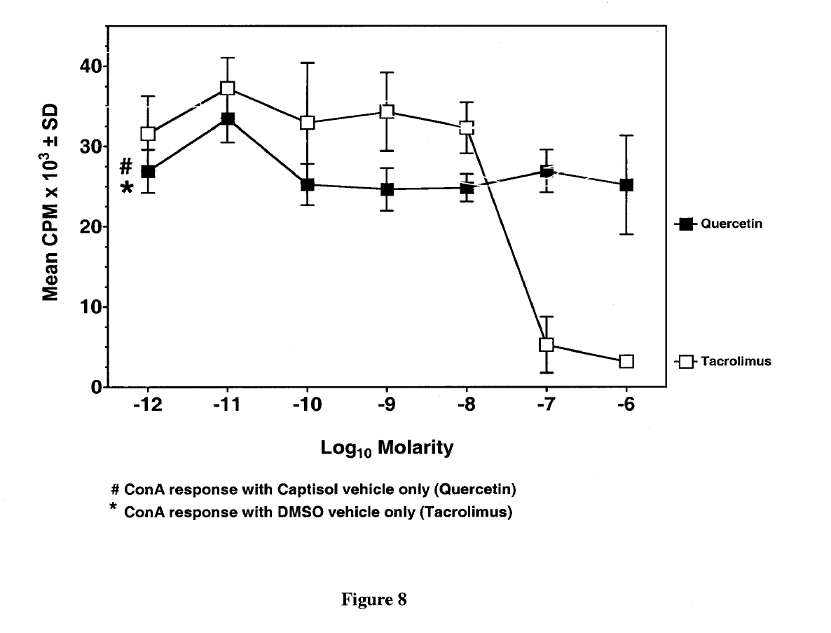

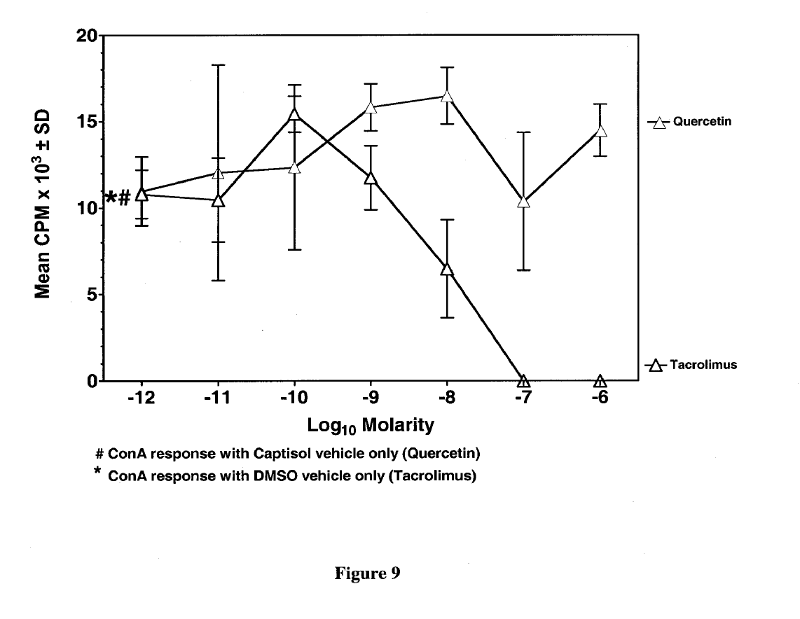

Results: FIGS. 8 and 9 show the effect of quercetin and tacrolimus on response of mouse spleen cells to Con A at a high (1.6×10⁶ cells/well) and low (8×10⁵ cells/well) cell concentration, respectively. As expected, tacrolimus inhibited Con A-induced proliferation in a dose dependent manner in cultures at high and low cell concentration. Quercetin had no significant effect in the response of cells to Con A at a high or low cell concentration.

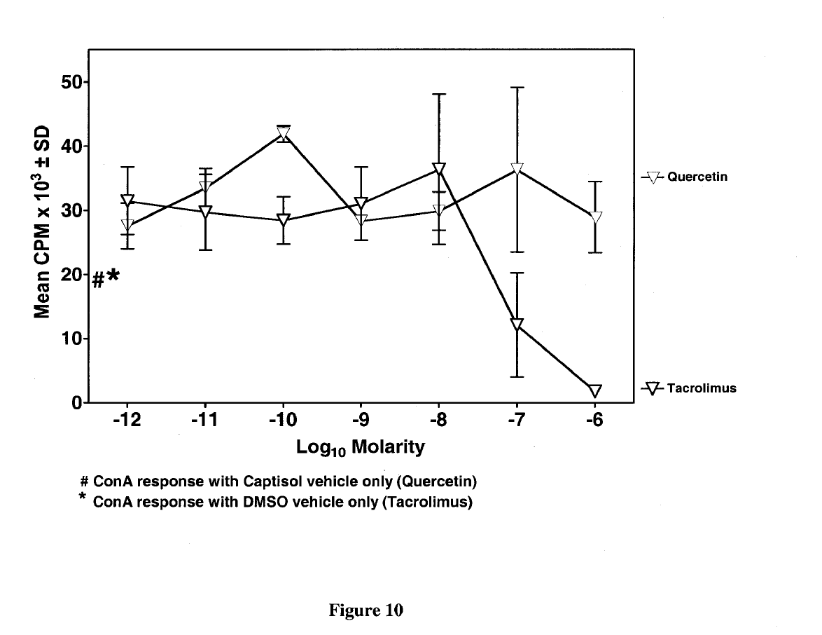

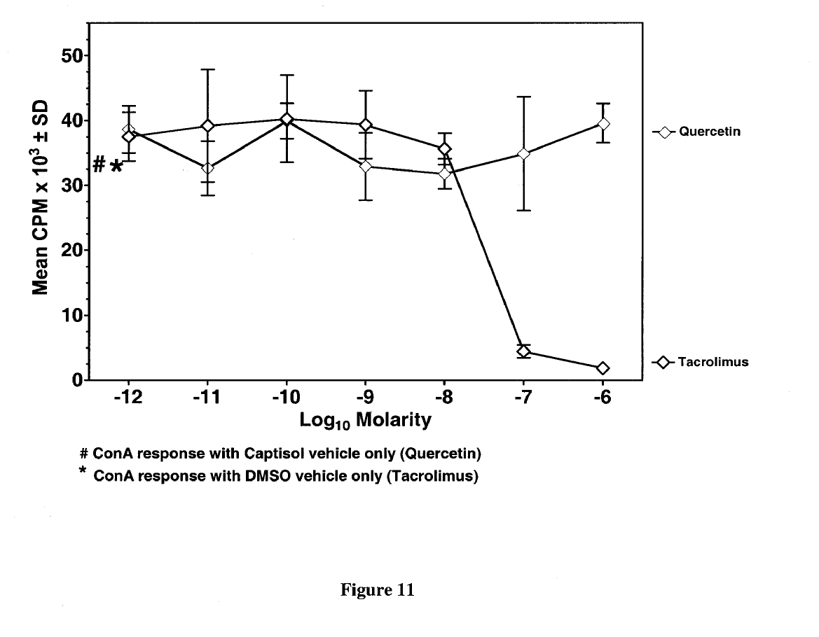

FIGS. 10 and 11 show the effect of quercetin and tacrolimus on response of mouse spleen cells to LPS at a high (1.6×10⁶ cells/well) and low (8×10⁵ cells/well) cell concentration, respectively. Like in the Con A assay, tacrolimus inhibited LPS-induced proliferation in a dose dependent manner both in high and low cell concentration cultures. Quercetin had no significant effect in the response of cells to LPS at a high or low cell concentration.

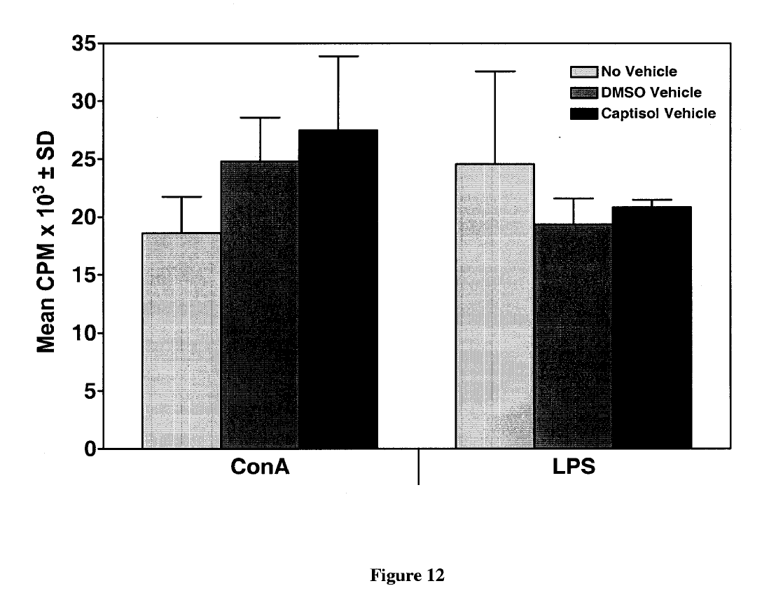

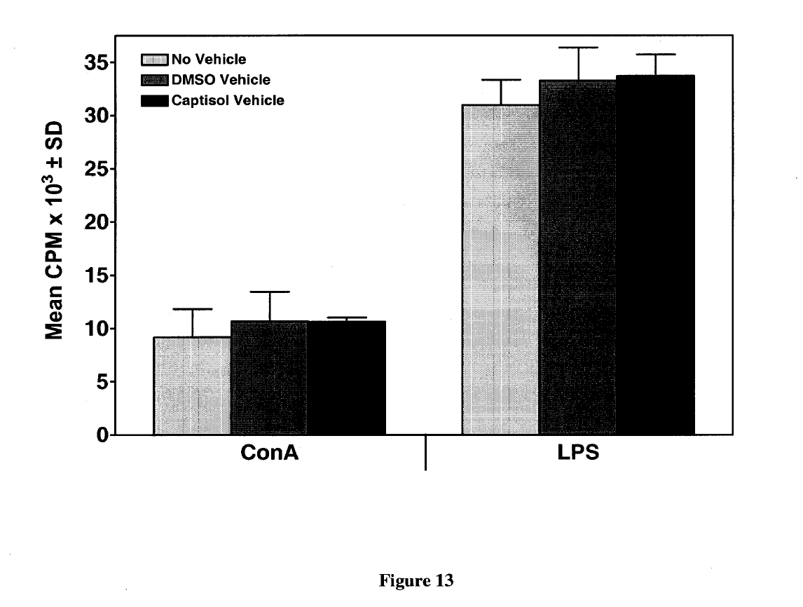

FIGS. 12 and 13 show the effect of vehicle treatment on mitogen responses at a high (1.6×10⁶ cells/well) and low (8×10⁵ cells/well) cell concentration, respectively. FIGS. 12 and 13 show no significant difference between no vehicle and either DMSO or captisol vehicle for either mitogen.

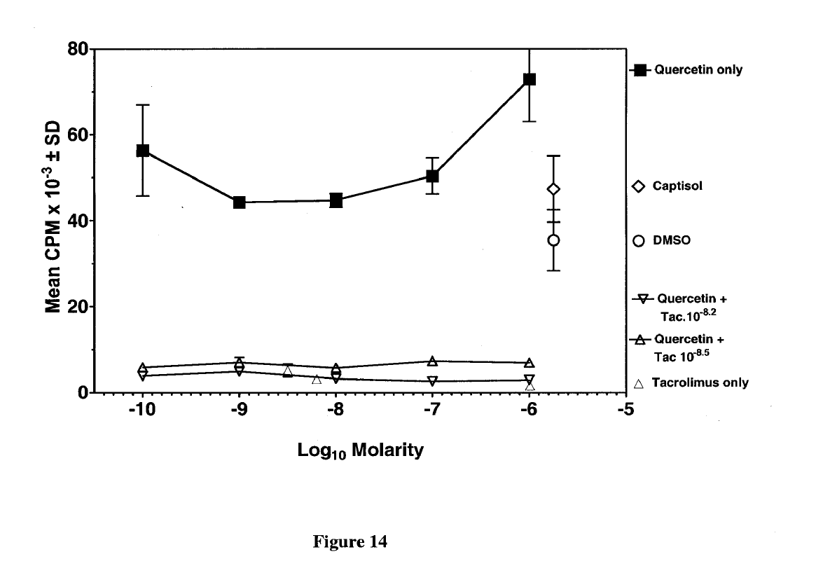

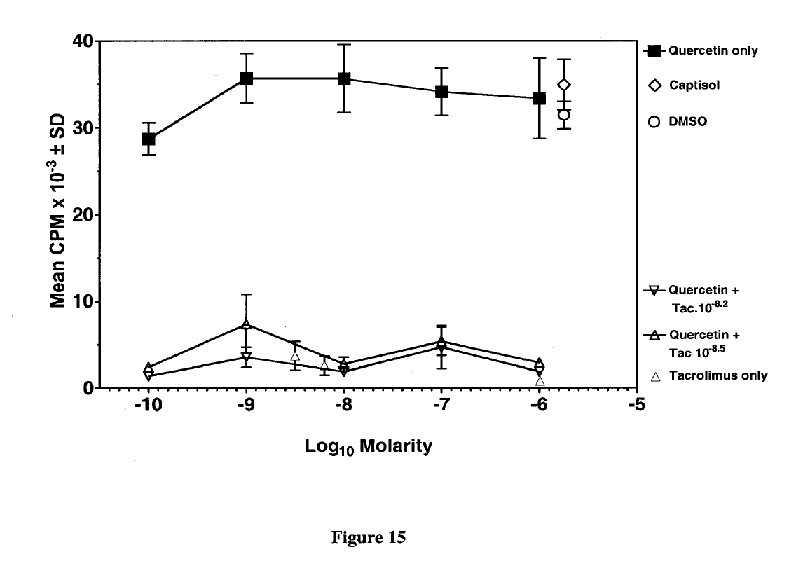

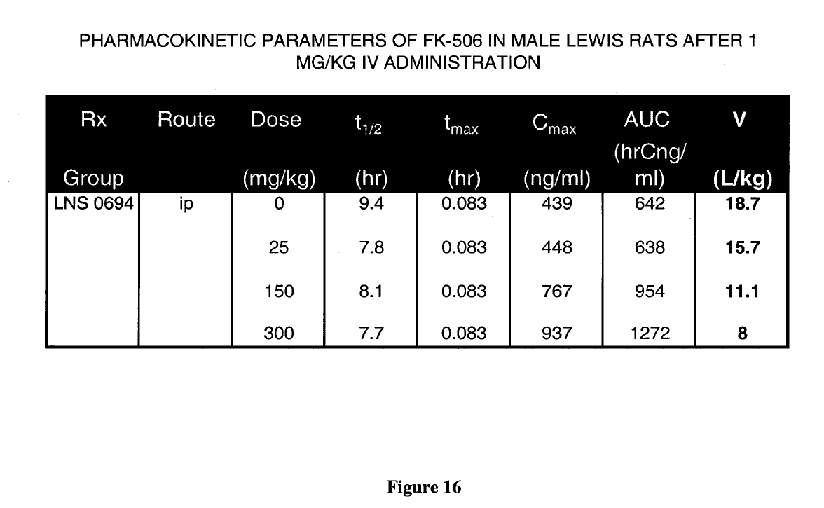

Spleen cells were treated with Con A in the presence of vehicle, tacrolimus, quercetin or two different concentrations of tacrolimus (10^{-8.2} and 10^{-8.5} M) and increasing doses of quercetin at a high cell concentration (FIG. 14) or at a low cell concentration (FIG. 15). FIG. 16 shows no significant difference between cultures treated with tacrolimus and cultures treated with tacrolimus and quercetin. Quercetin did not impair tacrolimus induced cell suppression in vitro at either concentration of tacrolimus. There was no significance difference between the effect of quercetin on tacrolimus induced cell suppression in vitro at the different concentrations used for quercetin. The same results were observed in cultures with low cell concentration (FIG. 15).

Taken together these results demonstrate that quercetin does not alter the effect of tacrolimus on cells.

### Example 5

### BTB Transport Protein Modulator Increase Peripheral Bioavailability and Decrease the Volume of distribution of Tacrolimus

Animals: 8-9 weeks-old Lewis and Brown Norway male rats were obtained from Charles River Laboratories. General procedures for animal care and housing was in accordance with the National Research Council (NRC) Guide for the Care and Use of Laboratory Animals (1996) and the Animal Welfare Standards incorporated in 9 CFR Part 3, 1991.

Treatment: Lewis rats were treated as described in example 3. Subsets of 3 Lewis rats per group were used for blood sampling at each time point (Groups 2-5) at described in the table below.

Pharmacokinetics Group (n = 9)

| Parameter: | 3 rats/group | 3 rats/group | 3 rats/group |
|---|---|---|---|
| Pharmacokinetics | 5 min | 15 min | 30 min |
| (whole blood | 1 hr | 4 hr | 8 hr |
| drug levels) | 2 hr | 6 hr | 24 hr |
| Penetration of | 2 hr | — | — |
| Blood-Tissue | | | |
| barrier | | | |
| (brain drug | | | |
| levels) | | | |

The brains were harvested from one subset of 3 rats per group at 2 hr after FK506 treatment.

Plasma Blood Sample Collection: Whole blood was collected from the retro-orbital sinus under 60:40 CO₂:O₂ anesthesia using EDTA as the anticoagulant. Sample collection was performed at 9 time points: 5, 15, and 30 min and 1, 2, 4, 6, 8, & 24 hr after administration of FK 506. Three samples were collected from each rat per group (Groups 2-5). The first 2 samples were collected under anesthesia, after which the animal regained consciousness and was retained until the next collection interval. The third/last sample was also collected under anesthesia, however the animal was euthanatized prior to anesthetic recovery.

Whole blood with EDTA anticoagulant was collected from 3 naïve rats and stored frozen at 80° C. (10° C.). These samples served as baseline PK samples. Additional blood samples were collected approximately 4 days prior to study start for method development.

Whole blood samples with EDTA anticoagulant were collected from 4 rats (to total ≧25 ml), stored on wet ice, and delivered to PK staff for method development. Brains were collected from 3 of these rats immediately following blood collection.

Whole blood samples without an anticoagulant were collected from a different set of 2 rats and processed to obtain a total ≧7 ml of serum. Serum samples were stored on wet ice and developed as described below. The sample volume was 500 μL.

Whole blood samples were placed on dry ice after collection and stored frozen at 80° C. (±10° C.) until analysis.

Drug levels were determined in collected whole blood samples using a bioanalytical method developed to detect parent drug levels.

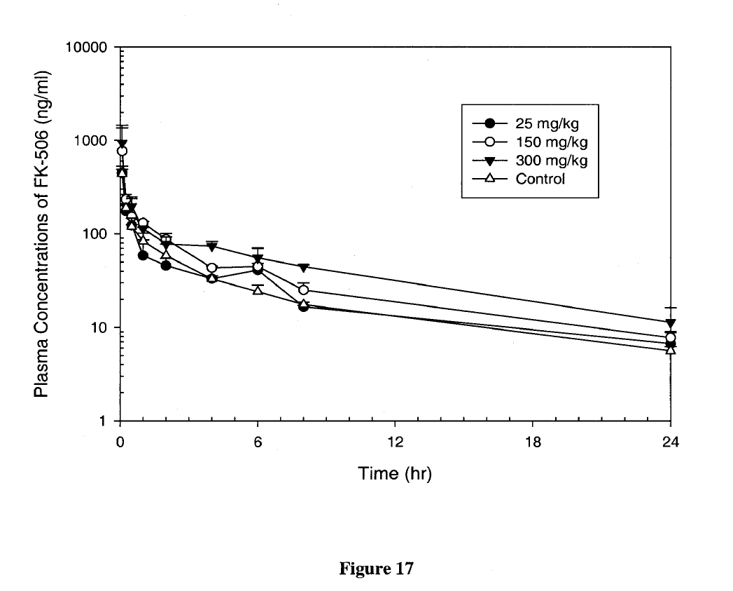

Results are shown in FIGS. 16 and 17.

Results: The pharmacokinetics parameters of FK 506 were determined in male Lewis rats after 1 mg/kg i.v. administration alone or in combination with i.p, administration of different doses of LNS 0694 (See FIG. 16).

FIG. 17 shows the plasma concentration of FK 506 at different time points after 1 mg/kg i.v. administration alone or in combination with i.p, administration of different doses of LNS 0694

Results in FIGS. 16 and 17 demonstrate that LNS 0694 increases the peripheral bioavailability of FK506 in a dose dependent manner.

### Example 6

### BTB Transport Protein Modulator Increase Peripheral Bioavailability and Decrease the Volume of Distribution of Tacrofimus

Animals: 8-9 weeks-old rats, e.g., Lewis and Brown Norway male rats, can be used. General procedures for animal care and housing are in accordance with the National Research Council (NRC) Guide for the Care and Use of Laboratory Animals (1996) and the Animal Welfare Standards incorporated in 9 CFR Part 3, 1991.

Treatment: Rats are treated as i.v with FK506 and i.p. with Quercetin at two different concentrations, 500 mg/kg or 200 mg/kg. Subsets of 3 Lewis rats per group are used for blood sampling at each time point (Groups 2-5) at described in the table below.

Pharmacokinetics Group (n = 9)

| Parameter: | 3 rats/group | 3 rats/group | 3 rats/group |
|---|---|---|---|
| Pharmacokinetics | 5 min | 15 min | 30 min |
| (whole blood | 1 hr | 4 hr | 8 hr |
| drug levels) | 2 hr | 6 hr | 24 hr |
| Concentration | 2 hr | — | — |
| of liver, | | | |
| pancreas, | | | |
| kidneys, | | | |
| intestines | | | |

The livers, pancreas, kidneys, and intestines are harvested from one subset of 3 rats per group at 2 hr after FK506 treatment.

Plasma Blood Sample Collection: Whole blood is collected from the retro-orbital sinus under 60:40 CO₂:O₂ anesthesia using EDTA as the anticoagulant. Sample collection is performed at 9 time points: 5, 15, and 30 min and 1, 2, 4, 6, 8, & 24 hr after administration of FK 506. Three samples were collected from each rat per group (Groups 2-5). The first 2 samples are collected under anesthesia, after which the animal regained consciousness and is retained until the next collection interval. The third/last sample is also collected under anesthesia, however the animal is euthanatized prior to anesthetic recovery.

Whole blood with EDTA anticoagulant is collected from 3 naïve rats and stored frozen at 80° C. (±10° C.). These samples can serve as baseline PK samples. Additional blood samples are collected approximately 4 days prior to study start for method development.

Whole blood samples with EDTA anticoagulant are collected from 4 rats (to total ≧25 ml), stored on wet ice, and delivered to PK staff for method development. Organs are collected from 3 of these rats immediately following blood collection.

Whole blood samples without an anticoagulant are collected from a different set of 2 rats and processed to obtain a total ≧7 ml of serum. Serum samples are stored on wet ice and developed as described below. The sample volume was 500 μL.

Whole blood samples are placed on dry ice after collection and stored frozen at 80° C. (±10° C.) until analysis.

Drug levels are determined in collected samples using bioanalytical methods developed to detect parent drug levels known in the art.

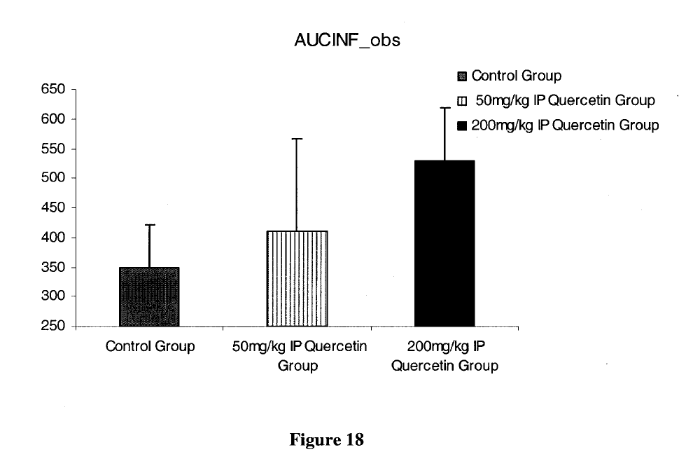

Results are shown in FIGS. 18, 19 and 20.

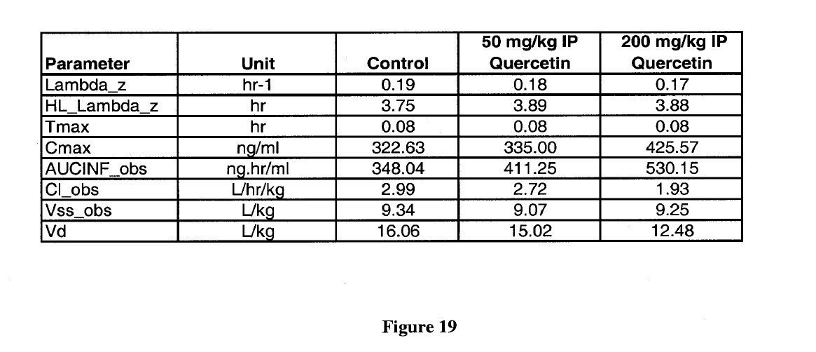

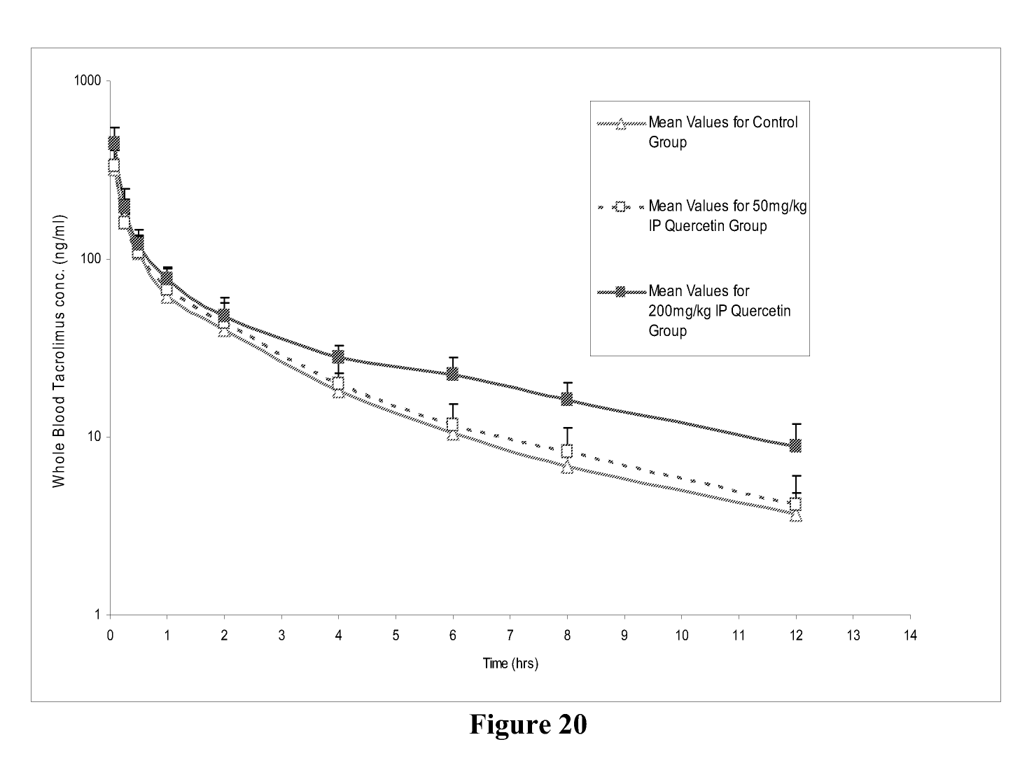

Results: FIG. 18 shows that the calculated AUC (0-infinity) increases after i.p. administration of different doses of Quercetin in a dose dependent manner when compared with the control group. FIG. 19 demonstrates an interaction at the higher 200 mg/kg quercetin dose including change in the Vd, AUC, Cmax and clearance. At this higher concentration of quercetin the Cmax and AUC increase while the Vd and clearance decrease. FIG. 20 shows the whole blood concentration of FK 506 at different time points after i.v. administration alone or in combination with i.p. administration of different doses of Quercetin. Results in FIGS. 18, 19 and 20 demonstrate that Quercetin increases the peripheral bioavailability of FK506 in a dose dependent manner.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising an effective amount of a calcineurin inhibitor and an amount of a BTB transport protein modulator sufficient to reduce a side effect of the calcineurin inhibitor.

2. A pharmaceutical composition comprising the composition of claim 1 and a pharmaceutically acceptable carrier.

3. The composition of claim 1 wherein said calcineurin inhibitor is tacrolimus.

4. The composition of claim 1 wherein said calcineurin inhibitor is a tacrolimus analog.

5. The composition of claim 4 wherein said tacrolimus analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

6. The composition of claim 1 wherein the BTB transport protein is an ABC transport protein.

7. The composition of claim 6 wherein the ABC transport protein is a P-gP.

8. The composition of claim 1 wherein said BTB transport protein modulator is a flavonoid or flavonoid derivative.

9. The composition of claim 8 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

10. The composition of claim 9 wherein said flavonoid or flavonoid derivative is quercetin or a quercetin derivative.

11. The composition of claim 1 wherein said calcineurin inhibitor is tacrolimus and said flavonoid or flavonoid derivative is quercetin.

12. The composition of claim 11 wherein tacrolimus and quercetin are present in a molar ratio of about 0.001:1 to 10:1.

13. The composition of claim 11 wherein tacrolimus is present at about 0.1-1000 mg and the quercetin is present at about 10-1000 mg.

14. The composition of claim 13 wherein said tacrolimus is present at about 0.5-100 mg and the quercetin is present at about 50-500 mg.

15. The composition of claim 14 wherein tacrolimus is present at about 5 mg and quercetin is present at about 500 mg.

16. The composition of claim 1 wherein a side effect of calcineurin inhibitor is decreased at least about 5% compared to the side effect without the BTB transport protein modulator, when the composition is administered to an animal.

17. The composition of claim 1 wherein said side effect is selected from the group consisting of CNS side effects, renal and/or urogenital side effects and hepatic, pancreatic and/or gastrointestinal side effects.

18. The composition of claim 17 wherein said side effect is a CNS side effect.

19. The composition of claim 18 wherein said CNS side effect is selected from the group consisting of tremors, headache, changes in motor function, changes in mental status, changes in sensory functions, seizures, insomnia, paresthesia, dizziness, coma and delirium.

20. The composition of claim 17 wherein said side effect is a hepatic, pancreatic and/or gastrointestinal side effect.

21. The composition of claim 20 wherein said hepatic, pancreatic and/or gastrointestinal side effect is hepatic necrosis, hepatotoxicity, liver fatty, venooclusive liver disease, diarrhea, nausea, constipation, vomiting, dyspepsia, anorexia, or LFT abnormal.

22. The composition of claim 17 wherein said side effect is a renal and/or urogenital side effect.

23. The composition of claim 22 wherein said renal and/or urogenital side effect side effect is nephrotoxicity, renal function impairment, creatinine increase, urinary tract infection, oliguria, cystitis haemorrhagic, hemolytic-uremic syndrome or micturition disorder.

24. The composition of claim 1 wherein said side effect is a decrease in tissue metabolic function.

25. A kit comprising the composition of claim 1 and instructions for use of the composition.

26. A method of decreasing a side effect of treatment with a calcineurin inhibitor comprising administering to a subject receiving treatment with said calcineurin inhibitor an amount of a BTB transport protein modulator sufficient to reduce said side effect in combination with said calcineurin inhibitor.

27. The method of claim 26 wherein said BTB transport protein modulator is a BTB protein transport activator.

28. The method of claim 26 wherein said side effect is selected from the group consisting of CNS side effects, renal and/or urogenital side effects and hepatic, pancreatic and/or gastrointestinal side effects.

29. The method of claim 28 wherein said side effect is a CNS side effect.

30. The method of claim 29 wherein said CNS side effect is selected from the group consisting of tremors, headache, changes in motor function, changes in mental status, changes in sensory functions, seizures, insomnia, paresthesia, dizziness, coma and delirium.

31. The method of claim 28 wherein said side effect is a hepatic, pancreatic and/or gastrointestinal side effect.

32. The method of claim 31 wherein said hepatic, pancreatic and/or gastrointestinal side effect is hepatic necrosis, hepatotoxicity, liver fatty, venooclusive liver disease, diarrhea, nausea, constipation, vomiting, dyspepsia, anorexia, or LFT abnormal.

33. The method of claim 28 wherein said side effect is a renal and/or urogenital side effect.

34. The method of claim 33 wherein said renal and/or urogenital side effect side effect is nephrotoxicity, renal function impairment, creatinine increase, urinary tract infection, oliguria, cystitis haemorrhagic, hemolytic-uremic syndrome or micturition disorder.

35. The method of claim 26 wherein said side effect is a decrease in tissue metabolic function.

36. The method of claim 26 comprising administering to a subject receiving treatment with said calcineurin inhibitor an amount of a BTB transport protein modulator sufficient to reduce a plurality side effect in combination with said calcineurin inhibitor.

37. The method of claim 26 wherein said BTB transport protein is an ABC transport protein.

38. The method of claim 37 wherein said ABC transport protein is P-gP.

39. The method of claim 26 wherein said BTB transport protein modulator is a flavonoid or flavonoid derivative.

40. The method of claim 39 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

41. The method of claim 40 wherein said flavonoid or flavonoid derivative is quercetin or a quercetin derivative.

42. The method of claim 26 wherein said calcineurin inhibitor is tacrolimus.

43. The method of claim 26 wherein said calcineurin inhibitor is a tacrolimus analog.

44. The method of claim 43 wherein said FK-506 analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

45. The method of claim 26 wherein said subject suffers from a condition selected from the group consisting of organ transplant, an autoimmune disease, and an inflammatory disease.

46. The method of claim 45 wherein said condition is organ transplant.

47. The method of claim 46 wherein said organ transplant is selected from the group consisting of kidney transplant, pancreas transplant, liver transplant, heart transplant, lung transplant, intestine transplant, pancreas after kidney transplant, and simultaneous pancreas-kidney transplant.

48. The method of claim 45 wherein said condition is an autoimmune disease.

49. The method of claim 48 wherein said autoimmune disease is selected from the group consisting of Lupus nephritis, actopic dermatitis, and psoriasis.

50. The method of claim 45 wherein said condition is an inflammatory disease.

51. The method of claim 50 wherein said inflammatory disease is selected from the group consisting of asthma, vulvar lichen sclerosis, chronic allergic contact dermatitis, eczema, vitiligo and ulcerative colitis.

52. The method of claim 26 wherein said administration comprises single or multiple doses of said calcineurin inhibitor and single or multiple doses of said BTB transport protein modulator.

53. The method of claim 26 wherein said administration comprising simultaneous administration of said calcineurin inhibitor and said BTB transport protein modulator in the same dosage form, simultaneous administration in separate dosage forms, or separate administration.

54. The method of claim 53 wherein said administration comprising simultaneous administration of said calcineurin inhibitor and said BTB transport protein modulator in the same dosage form.

55. The method of claim 26 wherein the molar ratio of the amount of the calcineurin inhibitor administered and the amount of BTB transport protein modulator administered is about 0.001:1 to about 10:1.

56. The method of claim 26 wherein the calcineurin inhibitor is administered in an amount sufficient to exert a therapeutic effect and the BTB transport protein modulator is administered in an amount sufficient to decrease a side effect of the calcineurin inhibitor by an average of at least about 5%, compared to the side effect without the BTB transport protein modulator.

57. The method of claim 56 wherein the BTB transport protein modulator is administered in an amount sufficient to increase the therapeutic effect of said calcineurin inhibitor by an average of at least about 5%, compared to the therapeutic effect without the BTB transport protein modulator.

58. The method of claim 56 wherein said BTB transport protein modulator is a BTB protein transport activator.

59. The method of claim 56 wherein said side effect is selected from the group consisting of CNS side effects, renal and/or urogenital side effects and hepatic, pancreatic and/or gastrointestinal side effects.

60. The method of claim 59 wherein said side effect is a CNS side effect.

61. The method of claim 60 wherein said CNS side effect is selected from the group consisting of tremors, headache, changes in motor function, changes in mental status, changes in sensory functions, seizures, insomnia, paresthesia, dizziness, coma and delirium.

62. The method of claim 59 wherein said side effect is a hepatic, pancreatic and/or gastrointestinal side effect.

63. The method of claim 62 wherein said hepatic, pancreatic and/or gastrointestinal side effect is hepatic necrosis, hepatotoxicity, liver fatty, venooclusive liver disease, diarrhea, nausea, constipation, vomiting, dyspepsia, anorexia, or LFT abnormal.

64. The method of claim 59 wherein said side effect is a renal and/or urogenital side effect.

65. The method of claim 64 wherein said renal and/or urogenital side effect side effect is nephrotoxicity, renal function impairment, creatinine increase, urinary tract infection, oliguria, cystitis haemorrhagic, hemolytic-uremic syndrome or micturition disorder.

66. The method of claim 56 wherein said side effect is a decrease in tissue metabolic function.

67. The method of claim 56 wherein said BTB transport protein is an ABC transport protein.

68. The method of claim 67 wherein said ABC transport protein is P-gP.

69. The method of claim 56 wherein said BTB transport protein modulator is a flavonoid or flavonoid derivative.

70. The method of claim 69 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

71. The method of claim 70 wherein said flavonoid or flavonoid derivative is quercetin or a quercetin derivative.

72. The method of claim 56 wherein said calcineurin inhibitor is tacrolimus.

73. The method of claim 56 wherein said calcineurin inhibitor is a tacrolimus analog.

74. The method of claim 73 wherein said FK-506 analog is selected from the group consisting of meridamycin, 31-O-Demethyl-FK506; L-683,590, L-685,818; 32-O-(1-hydroxyethylindol-5-yl)ascomycin; ascomycin; C18-OH-ascomycin; 9-deoxo-31-O-demethyl-FK506; L-688,617; A-119435; AP1903; rapamycin; dexamethasone-FK506 heterodimer; 13-O-demethyl tacrolimus; and FK 506-dextran conjugate.

75. A composition comprising an effective amount of a calcineurin inhibitor and an amount of a BTB transport protein modulator sufficient to increase a therapeutic effect of the calcineurin inhibitor.

76. (canceled)

77. The composition of claim 75 wherein said calcineurin inhibitor is tacrolimus.

78. The composition of claim 75 wherein said calcineurin inhibitor is a tacrolimus analog.

79. (canceled)

80. (canceled)

81. (canceled)

82. The composition of claim 75 wherein said BTB transport protein modulator is a flavonoid or flavonoid derivative.

83. The composition of claim 82 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

84. The composition of claim 83 wherein said flavonoid or flavonoid derivative is quercetin or a quercetin derivative.

85. (canceled)

86. (canceled)

87. (canceled)

88. (canceled)

89. (canceled)

90. The composition of claim 75 wherein a therapeutic effect of calcineurin inhibitor is increased at least about 5% compared to the therapeutic effect without the BTB transport protein modulator, when the composition is administered to an animal.

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. A composition comprising a therapeutically effective amount of a calcineurin inhibitor and an amount of a BTB transport protein modulator sufficient to change the concentration of the calcineurin inhibitor in a physiological compartment.

107. The composition of claim 106 wherein the BTB transport protein modulator is present in an amount sufficient to decrease the clearance of the calcineurin inhibitor from a physiological compartment in which the calcineurin inhibitor exerts a therapeutic effect.

108. (canceled)

109. The composition of claim 106 wherein said calcineurin inhibitor is tacrolimus.

110. The composition of claim 106 wherein said calcineurin inhibitor is a tacrolimus analog.

111. (canceled)

112. (canceled)

113. (canceled)

114. The composition of claim 106 wherein said BTB transport protein modulator is a flavonoid or flavonoid derivative.

115. The composition of claim 114 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

116. The composition of claim 115 wherein said flavonoid or flavonoid derivative is quercetin or a quercetin derivative.

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. A method of increasing a therapeutic effect of a calcineurin inhibitor comprising administering to a subject receiving treatment with said calcineurin inhibitor an amount of a BTB transport protein modulator sufficient to increase a therapeutic effect of the calcineurin inhibitor.

138. (canceled)

139. The method of claim 137 wherein the calcineurin inhibitor is administered in an amount sufficient to exert a therapeutic effect and the BTB transport protein modulator is administered in an amount sufficient to reduce a side effect of said calcineurin inhibitor.

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. The method of claim 137 wherein said BTB transport protein modulator is a flavonoid or flavonoid derivative.

152. The method of claim 151 wherein said flavonoid or flavonoid derivative is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

153. The method of claim 152 wherein said flavonoid or flavonoid derivative is quercetin or a quercetin derivative.

154. The method of claim 137 wherein said calcineurin inhibitor is tacrolimus.

155. The method of claim 137 wherein said calcineurin inhibitor is a tacrolimus analog.

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. The method of claim 137 wherein the calcineurin inhibitor is administered in an amount sufficient to exert a therapeutic effect and the BTB transport protein modulator is administered in an amount sufficient to change the concentration of calcineurin in a physiological compartment.

171. (canceled)

172. The method of claim 170 further comprising decreasing the clearance of the calcineurin inhibitor from a physiological compartment in which the calcineurin inhibitor is exerting its therapeutic effect.

173. (canceled)

174. (canceled)

175. (canceled)

176. (canceled)

177. (canceled)

178. (canceled)

179. (canceled)

180. (canceled)

181. (canceled)

182. (canceled)

183. (canceled)

184. (canceled)

185. (canceled)

186. (canceled)

187. (canceled)

188. (canceled)

189. (canceled)

190. A method of changing the concentration of tacrolimus or a tacrolimus analog in a physiological compartment comprising administering to a subject in need of treatment with said of tacrolimus or tacrolimus analog an amount of a BTB transport protein modulator sufficient to change the concentration of tacrolimus or a tacrolimus analog in a physiological compartment.

191. The method of claim 190 wherein said BTB transport protein modulator increases the concentration of tacrolimus or a tacrolimus analog in a physiological compartment.

192. The method of claim 191 wherein said physiological compartment is selected from the group consisting of blood, lymph nodes, spleen, peyer's patches, lungs, and heart.

193. The method of claim 190 wherein said BTB transport protein modulator decreases the concentration of tacrolimus or a tacrolimus analog in a physiological compartment.

194. The method of claim 193 wherein said physiological compartment is selected from the group consisting liver, intestines, kidney, lungs, heart, and gull bladder.

195. (canceled)

196. (canceled)

197. (canceled)

198. (canceled)

199. (canceled)

200. (canceled)

201. (canceled)

202. The composition of claim 1 wherein a therapeutic effect of the calcineurin inhibitor is increased at least about 5% compared to the therapeutic effect without the BTB transport protein modulator, when the composition is administered to an animal.
